(19) United States
(12) Patent Application Publication
(10) Pub. No.: US 2022/0241184 A1
(43) Pub. Date: Aug. 4, 2022

(54) GASTRORETENTIVE DOSAGE FORMS OF LEVODOPA AND CARBIDOPA

(71) Applicant: KASHIV BIOSCIENCES, LLC, Piscataway, NJ (US)

(72) Inventors: Kanji MEGHPARA, Morris Plains, NJ (US); Jaydeep VAGHASHIYA, Woodbridge, NJ (US); Dipen DESAI, Whippany, NJ (US); Wantanee PHUAPRADIT, Montville, NJ (US); Navnit H. SHAH, Clifton, NJ (US)

(21) Appl. No.: 17/620,824

(22) PCT Filed: Jun. 17, 2020
(86) PCT No.: PCT/US2020/038086

§ 371 Date: Dec. 20, 2021

(51) Int. Cl. A61K 9/00; A61K 31/198; A61K 9/20; A61K 9/28

(52) U.S. Cl. CPC A61K 9/0004; A61K 31/198; A61K 9/2846; A61K 9/2086; A61K 9/0065

(57) ABSTRACT

The present disclosure provides self-regulating, oral, osmotic, floating gastroretentive CD/LD compositions that are suitable for once- or twice-daily administration. The compositions provide extended release with enhanced pharmacokinetic attributes of LD, e.g., reduced lag time, avoidance of low trough levels, and reduced peak-to-trough ratios (Cₘₐₓ/Cₘᵢₙ) compared to marketed CD/LD products. The compositions provide extended release of CD/LD for about 8 to about 14 hours, without losing gastroretentive attributes of the system (GRS attributes), and collapse/squeeze after at least about 80% of the drug (CD/LD) is released from the system. The compositions of the disclosure, when consumed or when in contact with media simulating gastric conditions, float in about 45 minutes or less, swell in about 60 minutes or less to a swollen state that prevents their passage through the pyloric sphincter, and remain in the swollen state, while releasing steady therapeutic concentrations of the drug, for prolonged periods, e.g., about 8-14 hours.

### 1. RELATED APPLICATIONS

This application claims priority to U.S. Provisional Patent Application No. 62/865,039, filed Jun. 21, 2019 and U.S. Provisional Patent Application No. 62/867,731, filed Jun. 27, 2019, the disclosures of which are hereby incorporated by reference herein in their entireties.

### 2. TECHNICAL FIELD

The present disclosure provides self-regulating, osmotic, floating gastroretentive compositions of levodopa (LD) and carbidopa (CD) [CD/LD compositions], suitable for once- or twice-daily administration. The compositions provide extended release with enhanced pharmacokinetic attributes of LD, e.g., reduced lag time, avoidance of low trough levels, and reduced peak-to-trough ratios (Cₘₐₓ/Cₘᵢₙ) compared to marketed CD/LD products. The compositions provide extended release of CD/LD for about 8 to about 14 hours, without losing gastroretentive attributes of the system (GRS attributes), and squeeze/collapse after substantial or complete release of the drug from the system. The compositions of the disclosure, when consumed or when in contact with media simulating gastric conditions, float in 45 minutes or less, swell in 60 minutes or less to a size that prevents their passage through the pyloric sphincter, and remain in the swollen state, while releasing therapeutic concentrations of the drug, for prolonged periods, e.g., about 8-14 hours.

### 3. BACKGROUND

Combinations of LD and CD are known in the art for treating symptoms of Parkinson's disease (PD). Unfortunately, many Parkinson disease patients who initially respond positively to LD eventually develop motor complications, including “off” periods (when medication has worn off and parkinsonian symptoms reemerge) and LD induced dyskinesias. These complications, due to narrowing of the therapeutic window, can be a major source of distress and disability for patients. As such, an important aspect of PD therapy development has been to reduce “off” time without inducing development of dyskinesias. Orally developed LD compositions provide fluctuating LD plasma levels and unpredictable motor responses.

DUOPA enteral suspension, an intraduodenal infusion therapy approved in the United States, demonstrates significantly reduced motor complications and reduced “off-time.” The experiences from DUOPA show that the maintenance of a steady therapeutic plasma concentrations of LD and the avoidance of low trough levels appear to be effective in reducing off-time, increasing “on” time without disabling dyskinesia, and reducing the severity of dyskinesia in comparison to standard oral formulations. However, such infusion therapies are extremely inconvenient to the patient.

The results of DUOPA infusion therapy provide a rationale for the development of a treatment that provides relatively steady therapeutic plasma concentrations of LD to optimize relief of PD symptoms and to minimize off-times and dyskinesia. There remains a need for extended release oral dosage forms that can provide relatively steady therapeutic plasma concentrations of LD to reduce off-times, prolong on-time for PD patients. Currently available extended release CD/LD compositions are meant to provide extended release of LD over prolonged periods of time, while maintaining steady therapeutic plasma levels of LD. However, Parkinson's disease (PD) patients on such extended release dosage forms wake up in the morning having little or no mobility (off-time) due to the wearing off of the dose taken the day/evening before. Once the previous dose has wom off, the patients are usually unwilling, or even unable, to wait for the extended period of time required for an extended release dosage form to deliver the necessary plasma levels of LD. While the use of an immediate release formulation of LD can reduce this “wait time,” the use of an immediate release formulation of LD requires more frequent dosing and is associated with more fluctuations in plasma LD concentrations. There remains a need for extended release oral dosage forms, suitable for once or twice daily administration, that can improve patient compliance by decreasing lag time and providing steady therapeutic plasma levels of LD by reducing peak-to-trough (Cₘₐₓ/Cₘᵢₙ) fluctuations during daily dosing. There remains a need for extended release oral dosage forms providing steady therapeutic plasma concentrations of LD that can reduce off-times, prolong on-time without disabling dyskinesia, for PD patients.

Additionally, as LD is absorbed mainly in proximal small intestine, gastric emptying plays an important role in determining plasma LD levels after intake of conventional oral formulation. Erratic gastric emptying is common in PD patients and likely contributes to fluctuations in LD plasma levels and unpredictable motor responses observed with orally dosed LD. Accordingly, there remains a need to develop gastroretentive oral dosage forms of LD that can avoid erratic fluctuations in LD plasma levels by providing a sustained release of LD in stomach of a patient. The present invention fills this void by providing self-regulating, oral, osmotic, floating gastroretentive CD/LD compositions that provide desired pharmacokinetic attributes, i.e., substantially steady therapeutic plasma levels of LD and CD over prolonged periods of time compared to marketed CD/LD compositions.

Specifically, the present invention provides self-regulating, oral, osmotic, floating gastroretentive CD/LD compositions that are suitable for once- or twice-daily administration and can provide steady plasma levels of LD during the dosing period for more consistent dopaminergic stimulation in the brain of PD patient and subsequent improvement in clinical symptoms. The gastroretentive LD compositions of the disclosure provide (1) steady therapeutic plasma levels of LD with reduced lag time, and (2) a longer continuous release of LD to sustain the therapeutic effects and lessen the wearing off effects of LD therapy.

### 4. SUMMARY

In certain embodiments, the disclosure provides an osmotic, floating gastroretentive dosage form comprising a multilayer core comprising a pull layer containing CD, LD, an acid, and a gas-generating agent; and a push layer, a permeable elastic membrane containing at least one orifice and surrounding the multilayer core, and an immediate release drug layer containing CD and LD and surrounding the permeable elastic membrane. The permeable elastic membrane comprises a copolymer of ethyl acrylate, methyl methacrylate, and trimethylammonioethyl methacrylate chloride (1:2:0.2) with a glass transition temperature of between 60° C. and 70° C., and at least one plasticizer. The plasticizer is present in an amount of from about 10 wt % to about 25 wt % of the copolymer weight, the gas generating agent is present in an amount of from about 10 wt % to about 50 wt % of the pull layer weight, and the orifice in the permeable elastic membrane is in fluid communication with the pull layer. The dosage form, when coming in contact with a dissolution medium, swells within 60 minutes or less to a swollen state that prevents its passage through pyloric sphincter, and collapses/squeezes for complete emptying through the pyloric sphincter, after at least about 80% of the CD and the LD is released.

In certain embodiments, the dosage form, when coming in contact with the dissolution medium comprising 0.001N HCl and about 10 mM NaCl, exhibits a volume gain of at least about 100% in about 60 minutes or less, a volume gain of at least about 150% in about 2 hours, and collapses/squeezes to a volume gain of less than 150% in about 22 hours, from the time of contact with the dissolution medium.

In certain embodiments, the dosage form when coming in contact with a dissolution medium comprising 0.001N HCl and about 10 mM NaCl, exhibits a volume gain of at least about 100% in about 60 minutes or less, a volume gain of at least about 200% in about 2 hours, and collapses/squeezes to a volume gain of less than 200% in about 22 hours, from the time of contact with the dissolution medium.

In certain embodiments, the dosage form, when coming in contact with a dissolution medium comprising 0.001N HCl and about 10 mM NaCl, exhibits a volume gain of at least about 100% in about 60 minutes or less, a volume gain of at least about 250% in about 2 hours, and collapses/squeezes to a volume gain of less than 250% in about 22 hours, from the time of contact with the dissolution medium.

In certain embodiments, the dosage form when coming in contact with a dissolution medium comprising 0.001N HCl and about 10 mM NaCl, exhibits a volume gain of at least about 100% in about 60 minutes or less, a volume gain of at least about 300% in about 2 hours, and collapses/squeezes to a volume gain of less than 300% in about 22 hours, from the time of contact with the dissolution medium.

In certain embodiments, the dosage form, when coming in contact with a dissolution medium comprising 0.001N HCl and about 10 mM NaCl, remains in the swollen state for at least about 8 hours, from the time of contact with the dissolution medium.

In certain embodiments, the dissolution medium comprises about 0.001N HCl and about 10 mM NaCl.

In certain embodiments, the at least one plasticizer is selected from the group consisting of triethyl citrate, triacetin, polyethylene glycol, propylene glycol, dibutyl sebacate, and mixtures thereof.

In certain embodiments, the acid is selected from the group consisting of succinic acid, citric acid, malic acid, fumaric acid, stearic acid, tartaric acid, boric acid, benzoic acid, and mixtures thereof.

In certain embodiments, the pull layer and the push layer each comprises at least one water-soluble hydrophilic polymer. In certain embodiments, the water-soluble hydrophilic polymer in the push layer is a polyethylene oxide polymer having an average molecular weight greater than or equal to 600,000 Da. In certain embodiments, the polyethylene oxide polymer in the push layer has an average molecular weight of about 600K Da, about 700K Da, about 800K Da, about 900K Da, about 1M Da, about 2M Da, about 3M Da, about 4M Da, about 5M Da, about 6M Da, about 7M Da, or intermediate values therein. In certain embodiments, the water-soluble hydrophilic polymer in the pull layer is a mixture of a polyethylene oxide polymer having an average molecular weight less than or equal to 1M Da and a polyethylene oxide polymer with an average molecular weight of greater than 1M Da. In certain embodiments,

the water-soluble hydrophilic polymer in the pull layer is a mixture of a polyethylene oxide polymer having an average molecular weight of about 7M Da and a polyethylene oxide polymer with an average molecular weight of about 200K Da. In certain embodiments, the polyethylene oxide polymer with an average molecular weight of about 7M Da and the polyethylene oxide polymer with an average molecular weight of about 200K Da are present in a weight ratio of between 1:99 and 10:90.

In certain embodiments, the gas generating agent is NaHCO3, CaCO3, or a mixture thereof.

In certain embodiments, the dosage form provides extended release of the CD and LD for a period of at least about 8 hours.

In certain embodiments, the disclosure provides an osmotic, floating gastroretentive dosage form comprising a multilayer core comprising a pull layer containing CD, LD, an acid, and a gas-generating agent; a push layer; and a permeable elastic membrane containing at least one orifice and surrounding the multilayer core; and an immediate release drug layer containing CD and LD and surrounding the permeable elastic membrane. The permeable elastic membrane comprises a copolymer of ethyl acrylate, methyl methacrylate, and trimethylammonioethyl methacrylate chloride (1:2:0.2) with a glass transition temperature of between 60° C. and 70° C., and at least one plasticizer. The plasticizer is present in an amount of from about 10 wt % to about 25 wt % of the copolymer weight, the gas generating agent is present in an amount of from about 10 wt % to about 50 wt % of the pull layer weight, and the orifice in the permeable elastic membrane is in fluid communication with the pull layer. The dosage form, when coming in contact with a dissolution medium comprising about 0.001N HCl and about 10 mM NaCl, floats in about 45 minutes or less, swells within 60 minutes or less to a swollen state that prevents its passage through pyloric sphincter, and remains in the swollen state for at least about 8 hours.

In certain embodiments, the pull layer and the push layer each comprises at least one water-soluble hydrophilic polymer. In certain embodiments, the water-soluble hydrophilic polymer in the push layer is a polyethylene oxide polymer having an average molecular weight greater than or equal to 600K Da. In certain embodiments, the water-soluble hydrophilic polymer in the pull layer is a mixture of a polyethylene oxide polymer having an average molecular weight less than or equal to 1M Da and a polyethylene oxide polymer with an average molecular weight of greater than 1M Da.

In certain embodiments, the disclosure provides an osmotic, floating gastroretentive dosage form comprising a multilayer core comprising a pull layer containing CD, LD, an acid, and a gas-generating agent; and a push layer; a permeable elastic membrane containing at least one orifice and surrounding the multilayer core; and an immediate release drug layer containing CD and LD and surrounding the permeable elastic membrane. The permeable elastic membrane comprises a copolymer of ethyl acrylate, methyl methacrylate, and trimethylammonioethyl methacrylate chloride (1:2:0.2) with a glass transition temperature of between 60° C. and 70° C., and at least one plasticizer. The plasticizer is present in an amount of from about 10 wt % to about 25 wt % of the copolymer weight, the gas generating agent is present in an amount of from about 10 wt % to about 50 wt % of the pull layer weight, and the orifice in the permeable elastic membrane is in fluid communication with the pull layer. The dosage form, when coming in contact with a dissolution medium comprising about 0.001N HCl and about 10 mM NaCl, exhibits a volume gain of at least about 200% in about 60 minutes or less, and collapse to a volume gain of 150% or less in about 22 hours, from the time of contact with the dissolution medium.

In certain embodiments, the pull layer further comprises polyethylene oxide polymer having an average molecular weight less than or equal to 1M Da and a polyethylene oxide polymer with an average molecular weight of greater than 1M Da. In certain embodiments, the push layer comprises a polyethylene oxide polymer with an average molecular weight of at least about 600K Da.

In certain embodiments, the disclosure provides an osmotic, floating gastroretentive dosage form comprising a multilayer core comprising a pull layer containing CD, LD, an acid, and a gas-generating agent; and a push layer; and a permeable elastic membrane containing at least one orifice and surrounding the multilayer core. The permeable elastic membrane comprises a copolymer of ethyl acrylate, methyl methacrylate, and trimethylammonioethyl methacrylate chloride (1:2:0.2) with a glass transition temperature of between 60° C. and 70° C., and at least one plasticizer. The plasticizer is present in an amount of from about 10 wt % to about 25 wt % of the copolymer weight, the gas generating agent is present in an amount of from about 10 wt % to about 50 wt % of the pull layer weight, and the orifice in the permeable elastic membrane is in fluid communication with the pull layer. The dosage form is a horizontally compressed oval shaped bilayer tablet comprising a long axis with at length of between about 12 mm and about 22 mm, and a short axis with a length of between about 8 mm and about 12 mm. In certain embodiments, the dosage form, when coming in contact with a dissolution medium comprising about 0.001N HCl and about 10 mM NaCl, swells within 60 minutes or less to a swollen state that prevents its passage through pyloric sphincter and remains in the swollen state for at least about 8 hours. In certain embodiments, the dosage form further comprises an immediate release drug layer containing CD and LD. In certain embodiments the immediate release drug layer surrounds the permeable elastic membrane.

In certain embodiments, the disclosure provides an osmotic, floating gastroretentive dosage form comprising a multilayer core comprising a pull layer containing CD, LD, an acid, and a gas-generating agent; and a push layer, and a permeable elastic membrane containing at least one orifice and surrounding the multilayer core. The permeable elastic membrane comprises a copolymer of ethyl acrylate, methyl methacrylate, and trimethylammonioethyl methacrylate chloride (1:2:0.2) with a glass transition temperature of between 60° C. and 70° C., and at least one plasticizer. The plasticizer is present in an amount of from about 10 wt % to about 25 wt % of the copolymer weight, the gas generating agent is present in an amount of from about 10 wt % to about 50 wt % of the pull layer weight, and the orifice in the permeable elastic membrane is in fluid communication with the pull layer. The dosage form, when coming in contact with a dissolution medium comprising about 0.001N HCl and about 10 mM NaCl, swells within 60 minutes or less to a swollen state that prevents its passage through pyloric sphincter, and collapses/squeezes for complete emptying through the pyloric sphincter, after at least about 80% of the drug is released.

In certain embodiments, the disclosure provides a method for treating Parkinson's disease by administering to a subject an osmotic, floating gastroretentive dosage form comprising a multilayer core comprising a pull layer containing CD, LD, an acid, and a gas-generating agent; and a push layer; a permeable elastic membrane containing at least one orifice and surrounding the multilayer core; and an immediate release drug layer containing CD and LD and surrounding the permeable elastic membrane. The permeable elastic membrane comprises a copolymer of ethyl acrylate, methyl methacrylate, and trimethylammonioethyl methacrylate chloride (1:2:0.2) with a glass transition temperature of between 60° C. and 70° C., and at least one plasticizer. The plasticizer is present in an amount of from about 10 wt % to about 25 wt % of the copolymer weight, the gas generating agent is present in an amount of from about 10 wt % to about 50 wt % of the pull layer weight, and the orifice in the permeable elastic membrane is in fluid communication with the pull layer. The dosage form, when coming in contact with gastric fluid, swells within 60 minutes or less to a swollen state that prevents its passage through pyloric sphincter, remains in the swollen state for at least about 8 hours, and collapses/squeezes for complete emptying through the pyloric sphincter, after at least about 80% of the Cd and the LD is released.

In certain embodiments, the disclosure provides a method for treating post-encephalitic parkinsonism by administering to a subject an osmotic, floating gastroretentive dosage form comprising a multilayer core comprising a pull layer containing CD, LD, an acid, and a gas-generating agent; and a push layer; a permeable elastic membrane containing at least one orifice and surrounding the multilayer core; and an immediate release drug layer containing CD and LD and surrounding the permeable elastic membrane. The permeable elastic membrane comprises a copolymer of ethyl acrylate, methyl methacrylate, and trimethylammonioethyl methacrylate chloride (1:2:0.2) with a glass transition temperature of between 60° C. and 70° C., and at least one plasticizer. The plasticizer is present in an amount of from about 10 wt % to about 25 wt % of the copolymer weight, the gas generating agent is present in an amount of from about 10 wt % to about 50 wt % of the pull layer weight, and the orifice in the permeable elastic membrane is in fluid communication with the pull layer. The dosage form, when coming in contact with gastric fluid, swells within 60 minutes or less to a swollen state that prevents its passage through pyloric sphincter, remains in the swollen state for at least about 8 hours, and collapses/squeezes for complete emptying through the pyloric sphincter, after at least about 80% of the CD and the LD is released.

In certain embodiments, the disclosure provides a method for treating post-encephalitic parkinsonism by administering to a subject an osmotic, floating gastroretentive dosage form comprising a multilayer core comprising a pull layer containing CD, LD, an acid, and a gas-generating agent; and a push layer; a permeable elastic membrane containing at least one orifice and surrounding the multilayer core; and an immediate release drug layer containing CD and LD and surrounding the permeable elastic membrane. The permeable elastic membrane comprises a copolymer of ethyl acrylate, methyl methacrylate, and trimethylammonioethyl methacrylate chloride (1:2:0.2) with a glass transition temperature of between 60° C. and 70° C., and at least one plasticizer. The plasticizer is present in an amount of from about 10 wt % to about 25 wt % of the amount of the copolymer weight, the gas generating agent is present in an amount of from about 10 wt % to about 50 wt % of the pull layer weight, and the orifice in the permeable elastic membrane is in fluid communication with the pull layer. The dosage form, when coming in contact with gastric fluid, swells within 60 minutes or less to a swollen state that prevents its passage through pyloric sphincter, remains in the swollen state for at least about 8 hours, and collapses/squeezes for complete emptying through the pyloric sphincter, after at least about 80% of the CD and the LD is released.

In certain embodiment, the disclosure provides a method for improving bioavailability of LD, the method comprising administering to a subject, an osmotic, floating gastroretentive dosage form comprising a multilayer core comprising a pull layer containing CD, LD, an acid, and a gas-generating agent; and a push layer; and a permeable elastic membrane containing at least one orifice and surrounding the multilayer core. The permeable elastic membrane comprises a copolymer of ethyl acrylate, methyl methacrylate, and trimethylammonioethyl methacrylate chloride (1:2:0.2) with a glass transition temperature of between 60° C. and 70° C., and at least one plasticizer. The plasticizer is present in an amount of from about 10 wt % to about 25 wt % of the copolymer weight, the gas generating agent is present in an amount of from about 10 wt % to about 50 wt % of the pull layer weight, and the orifice in the permeable elastic membrane is in fluid communication with the pull layer. The dosage form, when coming in contact with gastric fluid, swells within 60 minutes or less to a swollen state that prevents its passage through pyloric sphincter, remains in the swollen state for at least about 8 hours, and collapses/squeezes for complete emptying through the pyloric sphincter, after at least about 80% of the CD and the LD is released.

In certain embodiments, the disclosure provides a method for making an osmotic, floating gastroretentive dosage form, The method comprises: (a) making a pull layer blend comprising CD/LD co-granulates and an extragranular component, (b) making a push layer blend, (c) compressing the pull layer blend and the push layer blend into a multilayered tablet core, (d) coating the tablet core with a functional coat to provide a functional coated tablet core, (e) drilling an orifice into the functional coat to provide a functional coated tablet core containing an orifice in fluid communication with the pull layer, and (f) coating the functional coated tablet core containing an orifice with an immediate release drug layer comprising CD and LD and at least one binder. The CD/LD co-granulates comprise CD, LD, a polyethylene oxide polymer with an average molecular weight of less than or equal to 1M Da, a polyethylene oxide polymer with an average molecular weight of greater than 1M Da, at least one acid, at least one binder, and at least one stabilizing agent; the extragranular component comprises at least one gas generating agent; the push layer comprises at least one polyethylene oxide polymer with an average molecular weight of greater than or equal to 600K Da and at least one osmogen; and the functional coat comprises a copolymer of ethyl acrylate, methyl methacrylate, and trimethylammonioethyl methacrylate chloride (1:2:0.2) with a glass transition temperature of between 60° C. and 70° C., and at least one plasticizer.

### 5. BRIEF DESCRIPTION OF THE DRAWINGS

FIG. 1 provides a schematic representation of the gastroretentive dosage form, according to certain embodiments, illustrating a bilayer tablet core, comprising a Push layer and a Pull layer, Seal Coat-1 surrounding the tablet core, a Functional Coat comprising a permeable elastic membrane surrounding Seal Coat-1, Seal Coat-2 surrounding the Functional Coat, Drug layer over Seal Coat-2, a Cosmetic Coat over Drug layer, and an Orifice passing through Seal Coat-1, Functional Coat, and Seal Coat-2, wherein the Orifice is in fluid communication with the Pull layer.

FIG. 2 compares floating lag times of Tablet 1 and Tablet 2 in a dissolution medium comprising about 250 ml of pH 4.5 acetate buffer, using USP dissolution apparatus III—Biodis reciprocating cylinder, at about 25 dpm and about 37° C. Tablet 1 contained a coating weight gain of about 150 mg in its functional coat, and Tablet 2 contained a coating weight gain of about 200 mg in its functional coat. FIG. 2 demonstrates that Tablets 1 and 2, irrespective of their different coating weight gains, exhibit a floating lag time of 15 minutes or less, measured from the time of contact with the dissolution medium.

FIG. 3 compares volumetric swelling of Tablets 1 and 2 in a dissolution medium comprising about 200 ml of pH 4.5 acetate buffer, using a rotating bottle method, at about 15 rpm and about 37° C. Tablet 1 contained a coating weight gain of about 150 mg in its functional coat, and Tablet 2 contained a coating weight gain of about 200 mg in its functional coat. FIG. 3 shows volume gain, measured from the time of contact with the dissolution medium, of Tablets 1 and 2 over an 18-hour period. FIG. 3 demonstrates that Tablets 1 and 2 exhibit a volume gain of about 100% in less than 1 hour, e.g., about 30 minutes; volume gain of at least 125% in about 2 hours; volume gain of at least 300% in about 4 hours; maintain the volume gain of about 300% from about 4 hours to about 16 hours; and collapse/squeeze to about 200% volume gain in about 16 hours, measured with respect to the tablet volume at the time of contact with the dissolution medium.

FIG. 4 compares dissolution profiles of Levodopa (“LD”) from Tablets 1 and 2, in a dissolution medium comprising about 900 ml of pH 4.5 acetate buffer, using USP dissolution apparatus I—Custom Basket, at about 100 rpm and about 37° C. Tablet 1 contained a coating weight gain of about 150 mg in its functional coat, and Tablet 2 contained a coating weight gain of about 200 mg in its functional coat. FIG. 4 demonstrates that Tablets 1 and 2 exhibit less than 20% dissolution of LD in about 2 hours, measured from the time of contact with the dissolution medium.

FIG. 5 compares dissolution profiles of LD from Tablets 1 and 2, in a dissolution medium comprising about 200 ml of pH 4.5 acetate buffer, using Rotating Bottle method, at about 15 rpm and about 37° C. Tablet 1 contained a coating weight gain of about 150 mg in its functional coat, and Tablet 2 contained a coating weight gain of about 200 mg in its functional coat. FIG. 5 demonstrates that Tablets 1 and 2 exhibit less than 30% dissolution of LD in about 2 hours, measured form the time of contact with the dissolution medium.

FIG. 6 compares dissolution profiles of LD from Tablets 1 and 2, in a dissolution medium comprising about 250 ml of pH 4.5 acetate buffer, using USP III—Biodis Reciprocating Cylinder, at about 25 dpm and about 37° C. Tablet 1 contained a coating weight gain of about 150 mg in its functional coat, and Tablet 2 contained a coating weight gain of about 200 mg in its functional coat. FIG. 6 demonstrates that Tablets 1 and 2 exhibit less than 30% dissolution of LD in about 2 hours, measured from the time of contact with the dissolution medium.

FIG. 7 shows cyclic dissolution profile of LD from Tablet 1 and Tablet 2, using USP III—Biodis Reciprocating Cylinder, at about 25 dpm and about 37° C., with an initial dissolution in a dissolution medium comprising about 250 ml pH 4.5 acetate buffer, followed by dissolution in a dissolution medium comprising about 250 ml 0.01 N HCl, and final dissolution in a dissolution medium comprising about 250 ml pH 4.5 acetate buffer. Tablet 1 contained a coating weight gain of about 150 mg in its functional coat, and Tablet 2 contained a coating weight gain of about 200 mg in its functional coat. FIG. 7 demonstrates that Tablets 1 and 2 exhibit less than 30% dissolution of LD in about 2 hours, measured from the time of contact with the dissolution medium comprising pH 4.5 acetate buffer.

FIG. 8 compares dissolution profiles of LD from Tablet 5 (about 240 mg LD) and Tablet 6 (about 320 mg LD), in about 900 ml of a dissolution medium comprising about 0.001 N HCl and about 10 mM NaCl, using USP I—Custom Basket, at about 100 rpm and about 37° C. FIG. 8 demonstrates that Tablets 5 and 6 exhibit about 40% dissolution of LD in about 2 hours, measured from the time of contact with the dissolution medium.

FIG. 9 compares volumetric swelling of Tablet 5 (about 240 mg LD) and Tablet 6 (about 320 mg LD) in a dissolution medium comprising about 200 ml of an aqueous medium comprising sodium chloride, potassium chloride, calcium chloride, phosphate salts, citric acid, and sugar (light meal media), using Rotating Bottle method, at about 15 rpm and about 37° C. FIG. 9 shows volume gain of Tablet 5 and Tablet 6 over an 8-hour period. The figure demonstrates that Tablets 5 and 6 exhibit a volume gain of about 100% in about 3 hours, measured with respect to the tablet volume at the time of contact with the dissolution medium.

FIG. 10 shows pharmacokinetic profiles of LD from single dose oral administrations of Tablets 1 and 2. Tablet 1 contained about 54 mg of CD, about 200 mg of LD, and a coating weight gain of about 150 mg in its functional coat. Tablet 2 contained about 54 mg of CD, about 200 mg of LD, and a coating weight gain of about 200 mg in its functional coat. FIG. 10 demonstrates that single dose administrations of Tablets 1 and 2 provided LD plasma concentrations of at least 300 ng/ml for about 9 hours.

FIG. 11 shows pharmacokinetic profiles for LD from single oral dose administrations of Tablets 5 and 6. Tablet 5 contained about 240 mg of LD, about 64.80 mg of CD, and about 51.50 mg of PARTECK® M200. Tablet 6 contained about 320 mg of LD, about 86.40 mg of CD, and no PARTECK® M200. Tablets 5 and 6 contained a coating weight gain of about 150 mg in their functional coat and equinormal amounts of succinic acid and gas-generating agent (a mixture of sodium bicarbonate and calcium carbonate). FIG. 11 demonstrates that single dose administrations of Tablets 5 and 6 provided LD plasma concentrations of at least 500 ng/ml for about 10 hours. FIG. 11 further demonstrates that Tablets 5 and 6 provided about 30% increase in bioavailability compared to Tablets 1 and 2 (see, e.g., Tablets 1 and 2 in FIG. 10), and showed dose proportionality between the 240 mg and 320 mg tablet strengths.

FIG. 12 shows MRI scans in an open label, single-treatment, single period MRI study of Tablet 5 (CD/LD—about 60/240 mg tablet containing black iron oxide as a contrast agent) in a healthy subject under fed conditions. The study was designed to determine the fate of the tablet at 8, 10, 12, 16, and 24 hours (+30 minutes) post dose. FIG. 12 demonstrates that the push layer containing polyethylene oxide with dispersed contrast agent is being released from the tablet between 16 hours and 24 hours post dose.

FIG. 13 compares dissolution profiles of LD from Tablet 13 (about 150 mg functional coat wt gain) and Tablet 14 (about 200 mg functional coat wt gain), in about 900 ml of a dissolution medium comprising about 0.01 N HCl and about 150 mM NaCl, using USP I-Custom basket, at about 100 rpm and about 37° C. Tablets 13 and 14 contained equinormal amounts of succinic acid and gas-generating agent (a mixture of sodium bicarbonate and calcium carbonate). FIG. 13 demonstrates that Tablet 13 exhibits about 35% dissolution of LD in about 4 hours, and Tablet 14 exhibits about 17% dissolution of LD in about 4 hours, measured from the time of contact with the dissolution medium.

FIG. 14 compares gravimetric expansion of Tablets 13 and 14, in a dissolution medium comprising about 200 ml of about 0.001N HCl and about 10 mM NaCl, measured as % weight increase from the form at the time of contact with the dissolution medium, using Rotating Bottle method, at about 15 rpm and about 37° C. FIG. 14 demonstrates that Tablet 13 with about 150 mg functional coat weight gain exhibits about 127% weight gain in about 8 hours and Tablet 14 containing about 200 mg functional coat weight gain exhibits about 153% weight gain in about 8 hours.

FIG. 15 compares gravimetric expansion of Tablets 5 and 6, in a dissolution medium comprising about 200 ml of about 0.001N HCl and about 10 mM NaCl, measured as % weight increase from the time of contact with the dissolution medium, using Rotating Bottle method, at about 15 rpm and about 37° C. Tablet 5 contained about 240 mg of LD, about 64.80 mg of CD, and about 51.50 mg of PARTECK® M200. Tablet 6 contained about 320 mg of LD, about 86.40 mg of CD, and no PARTECK® M200. Tablets 5 and 6 contained equinormal amounts of succinic acid and gas-generating agent (a mixture of sodium bicarbonate and calcium carbonate); and contained a coating weight gain of about 150 mg in their Functional Coat. FIG. 15 demonstrates that Tablet 5 exhibits about 125% weight gain in about 8 hours and Tablet 6 exhibits about 112% weight gain in about 8 hours.

FIG. 16 compares volumetric swelling of Tablets 5 and 6 in about 200 ml of a dissolution medium comprising about 0.001N HCl and about 10 mM NaCl, measured with respect to the tablet volume at the time of contact with the dissolution medium, using Rotating Bottle method, at about 15 rpm and about 37° C. Tablets 5 and 6 contained equinormal amounts of succinic acid and gas-generating agent (a mixture of sodium bicarbonate and calcium carbonate); and contained a coating weight gain of about 150 mg in their Functional Coat. FIG. 16 depicts volume gain of Tablets 5 and 6 over a 22-hour period. FIG. 16 demonstrates that Tablets 5 and 6 exhibit a volume gain of about 100% in less than 1 hour, e.g., about 30 minutes; volume gain of about 200% in about 2 hours; and collapse/squeeze to about 100% volume gain in about 22 hours.

FIG. 17 compares gravimetric expansion of Tablets 13 and 14, in a dissolution medium comprising about 200 ml of about 0.001N HCl and about 10 mM NaCl, measured as % weight increase from the time of contact with the dissolution medium, using Rotating Bottle method, at about 15 rpm and about 37° C. Tablet 13 contained a functional coat weight gain of about 150 mg, based on the total weight of the tablet before the functional coating. Tablet 14 contained a functional coating weight gain of about 200 mg, based on the total weight of the tablet before the functional coating. FIG. 17 demonstrates that Tablet 13 exhibits about 127% weight gain in about 8 hours, about 161% wt gain in about 14 hour, about 108% wt gain in about 18 hours, and about 93% wt gain in about 22 hours; and Tablet 14 exhibits about 153% weight gain in about 8 hours, about 118% weight gain in about 14 hours, about 85% weight gain in about 18 hours, and about 72% weight gain in about 22 hours.

FIG. 18 compares volumetric swelling of Tablets 13 and 14 in about 200 ml of a dissolution medium comprising about 0.001N HCl and about 10 mM NaCl, measured with respect to the tablet volume at the time of contact with the dissolution medium, using Rotating Bottle method, at about 15 rpm and about 37° C. Tablet 13 contained a functional coat weight gain of about 150 mg, based on the total weight of the tablet before the functional coating. Tablet 14 contained a functional coating weight gain of about 200 mg, based on the total weight of the tablet before the functional coating. FIG. 18 shows volume gain of Tablets 13 and 14 over a 22-hour period. FIG. 18 demonstrates that Tablet 13 exhibits a volume gain of about 100% in less than 1 hour, about 200% volume gain from about 2 hours to about 18 hours; and collapses/squeezes to about 150% volume gain in about 22 hours. FIG. 18 further demonstrates that Tablet 14 exhibits a volume gain of about 100% in less than about 1 hour, at least about 200% volume gain from about 2 hours to about 18 hours, and collapses/squeezes to about 150% volume gain in about 22 hours.

FIG. 19 compares volumetric swelling of Tablets 17 and 18 in about 200 ml of a dissolution medium comprising about 0.001N HCl and about 10 mM NaCl, measured with respect to the tablet volume at the time of contact with the dissolution medium, using Rotating Bottle method, at about 15 rpm and about 37° C. Tablets 17 and 18 contained a functional coating weight gain of about 150 mg, based on the total weight of the tablet before the functional coating. FIG. 19 shows volume gain of Tablets 17 and 18 over a 22-hour period. FIG. 19 demonstrates that Tablets 17 and 18 exhibit a volume gain of at least about 100% in about 30 minutes; about 200% in about 1 hour; at least 300% from about 2 hours to about 14 hours; and collapse/squeeze to about 250% volume gain from about 14 hours to about 22 hours.

FIG. 20 compares gravimetric expansion of Tablets 19 and 20, in a dissolution medium comprising about 200 ml of about 0.001N HCl and about 10 mM NaCl, measured as % weight increase from the time of contact with the dissolution medium, using Rotating Bottle method, at about 15 rpm and about 37° C. Tablet 19 contained about 86.40 mg of CD and about 320.0 mg of LD; and Tablet 20 contained about 64.80 mg of CD and about 240.0 mg of LD. FIG. 20 demonstrates that Tablet 20 exhibits about 114% weight gain in 6 hours and 68% wt gain in about 22 hours; and Tablet 19 exhibits about 95% weight gain at about 6 hours and 68% wt gain in about 22 hours.

FIG. 21 compares volumetric swelling of Tablets 19 and 20 in about 200 ml of a dissolution medium comprising about 0.001N HCl and about 10 mM NaCl, measured with respect to the tablet volume at the time of contact with the dissolution medium, using Rotating Bottle method, at about 15 rpm and about 37° C. Tablet 19 contained about 86.40 mg of CD and about 320.0 mg of LD; and Tablet 20 contained about 64.80 mg of CD and about 240.0 mg of LD. Tablets 19 and 20 contained a functional coating weight gain of about 150 mg, based on the total weight of the tablet before the functional coating. FIG. 21 shows volume gain of Tablets 19 and 20 over a 22-hour period. FIG. 21 demonstrates that Tablets 19 and 20 exhibit a volume gain of at least 100% in about one hour; at least 200% in about 4 hours; about 250% in about 14 hours; and collapse/squeeze to about 100% volume gain in about 22 hours.

### 6. DETAILED DESCRIPTION

The present disclosure provides self-regulating, oral, osmotic, floating gastroretentive CD/LD compositions providing steady plasma concentrations of LD in PD patients. The CD/LD compositions of the disclosure provide reduced lag time, avoid low trough levels, and exhibit reduced peak-to-trough ratios (Cmax/Cmin) compared to marketed CD/LD products. Such narrowing of peak-to-trough ratios (Cmax/Cmin) ratios and decreasing lag time for the drug release reduces “off-times” and prolongs “on-time” for PD patients.

The self-regulating, osmotic, floating gastroretentive CD/LD compositions of the disclosure expand rapidly in about 60 minutes or less to a size that prevents its passage through pyloric sphincter, and remain in an expanded state for prolonged periods, e.g., about 8-14 hours. The osmotic, floating gastroretentive CD/LD compositions of the disclosure improve drug bioavailability by retaining the dosage form in the stomach for prolonged periods of time and extending the release of the drug in the stomach or upper GI tract. Such prolonged gastric retention, with extended release provided by the osmotic, floating gastroretentive CD/LD compositions of the disclosure, improves drug bioavailability, reduces drug waste, and improves drug solubility. Additionally, the sustained release of LD in the stomach avoids the effect of erratic gastric emptying that is common in PD patients, thereby minimizing fluctuations in LD plasma levels and unpredictable motor responses.

For clarity and not by way of limitation, this detailed description is divided into the following subportions:

6.1. Definitions;

6.2. Self-regulating, Oral, Osmotic, Floating Gastroretentive Dosage Forms;

6.3. Methods of Treating;

6.4. Methods of Making; and

6.5. Features of the Dosage Forms.

### 6.1. Definitions

The terminology used in the present disclosure is for the purpose of describing particular embodiments only and is not intended to be limiting. As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

As used herein, “and/or” refers to and encompasses any and all possible combinations of one or more of the associated listed items.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 15%, up to 10%, up to 5%, up to 1%, up to 0.5%, or even up to 0.1% of a given of a value.

As used herein, a “therapeutically effective,” “therapeutic,” or “therapeutically acceptable” amount refers to an amount that will elicit a therapeutically useful response in a subject and includes an additional amount or overage of active ingredient deemed necessary in the formulation to provide the desired amount upon administration. The therapeutically useful response can provide some alleviation, mitigation, and/or decrease in at least one clinical symptom in the subject. Those skilled in the art will appreciate that the therapeutically useful response need not be complete or curative, as long as some benefit is provided to the subject.

The terms “steady plasma concentration,” “steady plasma level,” “steady therapeutic plasma concentration,” and “steady therapeutic plasma level” as used interchangeably herein, refer to consistent plasma levels/concentrations of LD that will elicit a therapeutically useful response in a subject and includes an additional amount or overage of active ingredient deemed necessary in the formulation to provide the desired amount upon administration.

The terms “osmotic gastroretentive dosage form,” “self-regulating, osmotic, floating gastroretentive dosage form /,” or the like, refer to a self-regulating, push-pull osmotic, floating dosage form providing delayed gastric emptying as compared to food (e.g., retention in the stomach beyond the retention of food).

As used herein, the terms “treatment,” “treat,” and “treating” refer to reversing, alleviating, delaying the onset of, and/or inhibiting the progress of a disease or disorder as described herein. In some embodiments, treatment can be administered after one or more symptoms have developed. In other embodiments, treatment can be administered in the absence of symptoms. For example, treatment can be administered to a susceptible individual prior to the onset of symptoms (e.g., in light of a history of symptoms and/or in light of genetic or other susceptibility factors). Treatment can also be continued after symptoms have resolved, for example to prevent or delay their recurrence.

The term “self-regulating” as used herein refers to a gastroretentive dosage form that floats, expands, and finally collapses to allow emptying of the dosage form from the GI tract and the patient.

The terms “osmotic dosage form” and the like, as used herein, refer to a push-pull osmotic dosage form containing a pull layer and a push layer, wherein the push layer swells to push the pull layer through an orifice, out of the dosage form. In certain embodiments, the pull layer can comprise two or more layers.

The term “osmosis,” as used herein, refers to movement of a solvent from a solution of low solute concentration to a solute or a solution of high solute concentration through a semipermeable or permeable membrane. The term “osmotic agent” includes swellable hydrophilic polymers, and osmogens/ionic compounds consisting of inorganic salts.

The terms “active agent,” “active ingredient,” “active pharmaceutical agent,” “active pharmaceutical ingredient” and “drug,” as used interchangeably herein, refer to a combination of LD and CD (CD/LD) that provides a therapeutic or prophylactic effect in the treatment of Parkinson's disease (PD), post-encephalitic parkinsonism, and parkinsonism that may follow carbon monoxide intoxication or manganese intoxication.

The term “pharmaceutically acceptable,” when used in connection with the pharmaceutical compositions of the disclosed subject matter, refers to molecular entities and compositions that are physiologically tolerable and do not typically produce untoward reactions when administered to a human. As used herein, the term “pharmaceutically acceptable” can also refer to being approved by a regulatory agency of the Federal or a state government or listed in the U.S. Pharmacopeia, National Formulary and Drug Standard Laboratory (NF), or other generally recognized pharmacopeia for use in animals, and more particularly in humans.

The term “bioavailability,” as used herein refers to the fraction of an administered drug that reaches the systemic circulation, as measured through various pharmacokinetic metrics such as Cₘₐₓ, Tₘₐₓ, AUC₀₋ₜ, and AUC₀₋ᵢₙₜ.

The terms “dosage form,” “formulation,” “composition,” and “pharmaceutical composition,” as used interchangeably herein, refer to pharmaceutical drug products in the form in which they are marketed for use, with specific mixture of active pharmaceutical ingredients and inactive excipients, in a particular configuration, e.g., tablets, capsules, particles, and apportioned into a particular dose.

The term “simulated gastric fluid,” as used herein, refers to fluid medium that is used to mimic chemical environment of gastric medium in vitro.

The term “gastric fluid,” as used herein, refers to medium occurring in stomach of an individual.

The terms “dissolution medium” and “medium simulating gastric conditions,” as used interchangeably herein, refer to a biorelevant medium mimicking gastric fluid conditions. In certain embodiments, the dissolution medium comprises pH 4.5 acetate buffer; 0.01N HCl; about 0.001N HCl and about 10 mM NaCl; or 0.01N HCL with 150 mM NaCl. In certain embodiments, the biorelevant medium comprises a “light meal medium.”

The term “light meal medium,” as used herein, refers to medium simulating gastric medium of an individual after consumption of a light meal. The term “light meal medium” refers to an aqueous medium comprising sodium chloride, potassium chloride, potassium hydrogen phosphate, calcium chloride, citric acid, and sugar.

The term “degradable,” as used herein, refers to capable of being chemically and/or physically modified, dissolved, or broken down, e.g., in the body of a patient, within a relevant time period.

The term “prolonged period” or the like, as used herein, refers to a period that lasts for at least 8 hours, e.g., from about 8 hours to about 14 hours. A prolonged period includes 8, 9, 10, 11, 12, 13, 14, or more hours. In certain embodiments, a prolonged period can include up to 24 hours.

The terms “swellable” and “swelling,” as used herein, can be used interchangeably and refer to a tablet core or a polymer present in the tablet core that swells by imbibing fluid and/or trapping CO2.

The terms “expanding” and “expansion,” as used herein with respect to a membrane, can be used interchangeably and refer to stretching or distention of the membrane due to an outward pressure (e.g., gas pressure, or pressure due to swelling of a polymer in the core) on the membrane.

The terms “volume expansion” and “volume expansion percentage,” as used interchangeably herein, refer to % increase in volume of the dosage form, based on the volume of the dosage form at the time of contact with a dissolution medium.

The term “change in weight %,” as used herein refers to percentage change in the weight of the dosage form, based on the weight of the dosage form at the time of contact with a dissolution medium.

The terms “rapidly expanding” and “rapidly swelling,” as used interchangeably herein, with respect to a gastroretentive dosage form, refers to rapid expansion of the dosage form due to initial faster expansion of the membrane than swelling of the core due to imbibition of fluid and generation of CO2. In certain embodiments, the term “rapidly expanding” refers to expansion of the membrane to provide at least 100% increase in volume of the dosage form, based on the volume of the dosage form at the time of contact with a dissolution medium, in less than 60 minutes.

The terms “shear” and “shear effect,” as used interchangeably herein, refer to peristaltic waves moving from the midcorpus of the stomach to the pylorus, particularly in a fed state.

The terms “pore former” and the like, as used herein, refer to water-soluble polymers and/or water-soluble small molecules that will form pores or channels (i.e., behave as a channeling agent) in the functional coat/membrane, thereby increasing the permeability of the membrane. The term “pore former” includes molecules used to create a certain amount of diffusion through the semipermeable or permeable membrane to achieve a desired extended release profile.

The terms “permeable membrane,” and “permeable elastic membrane” as used interchangeably herein, refer to a polymeric elastic membrane/film that is substantially permeable to the passage of solutes and passage of fluids/solvents. The “permeable membrane” includes water-insoluble permeable polyacrylate/polymethacrylate copolymers (copolymers of ethyl acrylate, methyl methacrylate and trimethylammonioethyl methacrylate chloride) with Tg (glass transition temperatures) of between about 50° C. and about 70° C. In certain embodiments, the “permeable membrane” can include copolymers of ethyl acrylate, methyl methacrylate, and trimethylammonioethyl methacrylate chloride with Tg of between about 60° C. and about 70° C. (e.g., EUDRAGIT® RL PO). In certain embodiments, the permeable membrane can include copolymers of ethyl acrylate, methyl methacrylate, and trimethylammonioethyl methacrylate chloride (1:2:0.2) having Tg of between about 50° C. and about 70° C. In certain embodiments, the “permeable membrane” includes copolymers of ethyl acrylate, methyl methacrylate, and trimethylammonioethyl methacrylate chloride (1:2:0.2) having a Tg of between about 60° C. and about 70° C. (EUDRAGIT® RL PO). In certain embodiments, the “permeable membrane” includes copolymers of ethyl acrylate, methyl methacrylate, and trimethylammonioethyl methacrylate chloride (1:2:0.1) having a Tg of between about 60° C. and about 70° C. (EUDRAGIT® RS PO)

The term “semipermeable membrane,” as used herein, refers to a polymeric membrane or a film that is substantially impermeable to the passage of solutes, including drug and other excipients/ingredients and substantially permeable to passage of fluids/solvents. The semipermeable membrane can include various cellulosic polymers including cellulose ethers, cellulose esters and cellulose ester-ethers. The semipermeable membrane does not include permeable polyacrylate and/or polymethacrylate copolymers with a Tg of between 50° C. and 70° C.

The terms “polyacrylate copolymer” and “polymethacrylate copolymer,” as used interchangeably herein refer to copolymers of ethyl acrylate, methyl methacrylate and trimethylammonioethyl methacrylate chloride having a Tg (glass transition temperatures) of between about 50° C. and about 70° C.

The terms “glass transition temperature” or “Tg,” as used interchangeably herein, refer to a temperature at which the polymer structure turns viscous liquid or rubbery. It is also defined as a temperature at which amorphous polymer takes on characteristic glassy-state properties like brittleness, stiffness, and rigidity upon cooling.

The term “multilayered” tablets core as used herein, refers to a compressed tablet core comprising at least two layers. In certain embodiments, the “multilayered tablet core” is a “bilayered tablet core” comprising a push layer and a pull layer.

The term “substantially free,” as used herein, refers to excluding any functional (e.g., noncontaminating) amount, which refers to any amount that contributes or has an effect on the following properties of the dosage form: floating lag time, volume expansion, release profile, and lag time to drug release.

The terms “orifice” and “hole,” as used interchangeably herein include, but are not limited to, at least one opening/exit means in the coatings of the osmotic gastroretentive composition to provide fluid communication with the pull layer. The opening (basically a delivery port) can be formed via manual or laser drilling of the membrane coat and seal coats, often into the side facing the pull layer. The orifice/hole cannot be present in the immediate release (IR) drug layer, Cosmetic Coat/Over Coat, or Final Coat/Clear Coat.

The term “patient,” as used herein, refers to a human or nonhuman mammal that may need to receive an osmotic gastroretentive dosage form of the present disclosure.

The term “upper GI tract,” as used herein, refers to the stomach, and proximal parts of the small intestine, e.g., the duodenum and jejunum.

The term “lower GI tract,” as used herein, refers to distal parts of the small intestine, e.g., the ileum, and all of the large intestine, including the colon, cecum, and rectum.

The term “floating” or the like, and as used herein in conjunction with a “floating gastroretentive dosage form” or the like, refers to a dosage form that has a bulk density less than gastric fluid and simulated gastric fluid (SGF). Such dosage forms are “floating” in that they remain buoyant in the gastric fluids of the stomach or SGF for a targeted period of time.

The term “floating lag time,” as used herein, includes the time between the addition of a dosage form to a medium and the time when the dosage form begins to float on the surface of the medium (e.g., in an in vitro setting), or the time between the consumption of a dosage form by a user and the time when the dosage form begins to float on the surface of the gastric fluid (e.g., in an in vivo setting).

The term “dissolution lag time,” as used herein, refers to the time between the addition of a dosage form to a medium and the time when the active agent begins to dissolve in the medium.

The term “medium,” as used herein, refers to a dissolution medium in an in vitro setting and gastric fluid in an in vivo setting.

The term “viscosity gradient,” as used herein, refers to a difference in viscosity between adjacent layers of the multilayered gastroretentive dosage forms of the disclosure. The term “decreasing viscosity gradient,” as used herein, refers to a decrease in viscosity from the push layer to the pull layer, wherein the push layer and the pull layer are adjacent to each other; or a decrease in viscosity between adjacent pull layers.

The term “modified release,” as used herein, refers to dosage forms or compositions that are formulated to modify drug release and drug availability, after administration, over a desired period of time that is longer than a corresponding immediate release period, thereby allowing a reduction in dosing frequency. Modified release dosage forms or compositions can include, but are not limited to, “extended release,” “controlled release,” “controlled extended release,” “delayed release,” and “pulsatile release” dosage forms or compositions.

The terms “extended release,” “controlled release,” and “controlled extended release,” as used herein, can be used interchangeably and refer to modified release dosage forms or compositions that are formulated to provide and maintain targeted concentration of an administered drug, over an extended period of time after administration, as compared to a drug presented as an immediate release dosage form.

### 6.2. Self-Regulating, Oral, Osmotic, Floating Gastroretentive Dosage Forms

The present disclosure provides self-regulating, oral, osmotic, floating gastroretentive CD/LD compositions with enhanced pharmacokinetic attributes. The gastroretentive CD/LD compositions of the disclosure provide extended release of LD with reduced lag time, and narrow peak-to-trough ratios (Cₘₐₓ/Cₘᵢₙ) leading to steady LD plasma levels over extended periods of time. The gastroretentive compositions of the disclosure, due to the presence of a permeable elastic membrane and a push-pull osmotic core, can provide steady delivery of the moderately soluble drug, e.g., LD because the permeable elastic membrane may allow for gastric retention and passive diffusion of the drug, and the push-pull system may provide an additional thrust to expel the drug when drug concentration decreases over time.

The gastroretentive CD/LD compositions of the disclosure expand rapidly in 60 minutes or less to a size that prevents their passage through the pyloric sphincter and remain in an expanded state to provide extended release of CD and LD for prolonged periods, e.g., about 8-14 hours. The gastroretentive CD/LD compositions of the disclosure improve bioavailability of LD by retaining the dosage form in the stomach of a subject for prolonged periods of time and extending the release of CD and LD in the stomach or upper GI tract. Such prolonged gastric retention with extended release provided by the gastroretentive CD/LD compositions of the disclosure, improves drug bioavailability, reduces drug waste, and improves drug solubility. Additionally, the sustained release of LD in the stomach avoids/minimizes the effect of erratic gastric emptying, a condition common in PD patients, thereby minimizing fluctuations in LD plasma levels and unpredictable motor responses.

The gastroretentive CD/LD compositions of the disclosure comprise an advanced self-regulating, oral, osmotic, floating gastroretentive drug delivery system that, when in contact with gastric fluid, floats in 45 minutes or less, expands rapidly in about 60 minutes or less to a size that prevents its passage through pyloric sphincter, and remains in an expanded state for prolonged periods, e.g., about 8-14 hours. The gastroretentive compositions of the disclosure include: i) a swellable multilayered tablet core comprising a pull layer and a push layer; and ii) a rapidly expanding permeable elastic membrane surrounding the swellable core, wherein the membrane comprises a plasticizer and at least one copolymer of ethyl acrylate, methyl methacrylate, and trimethylammonioethyl methacrylate chloride (1:2:0.2) with a Tg of between about 60° C. and about 70° C. (EUDRAGIT® RL PO). In certain embodiments, the gastroretentive compositions further include an immediate release (IR) drug layer containing CD and LD. In certain embodiments, the IR drug layer is present over the permeable elastic membrane/functional coat. The gastroretentive CD/LD compositions of the disclosure rely on size and buoyancy of the dosage form to retain the dosage form in the stomach for extended periods of time. The compositions of the disclosure combine the advantages of a gastroretentive system and a push-pull osmotic system to provide about 8-14 hours of gastric retention with a steady plasma concentration of LD for at least the same periods of time.

In certain embodiments of the disclosure, the self-regulating, oral, osmotic, floating gastroretentive CD/LD composition, which swells in about 60 minutes or less to a size that prevents its passage through the pyloric sphincter, remains in the swollen state for at least 8 hours and then collapse/squeeze for emptying from the stomach, comprises: (i) a swellable multilayered tablet core comprising a pull layer comprising CD and LD, a gas-generating agent, at least one polyethylene oxide polymer having an average molecular weight of less than or equal to about 1M (million) Da, and at least one polyethylene oxide polymer with an average molecular weight of greater than 1M Da; and a push layer comprising at least one polyethylene oxide polymer having an average molecular weight of greater than or equal to about 600K (600,000) Da, and at least one osmogen; (ii) a permeable elastic membrane, containing an orifice/hole in fluid communication with the pull layer, over the multilayer tablet core, and comprising a plasticizer and a copolymer of ethyl acrylate, methyl methacrylate, and trimethylammonioethyl methacrylate chloride (1:2:0.2) with a Tg of between about 60° C. and about 70° C. (EUDRAGIT® RL PO); and (iii) an IR drug layer containing CD and LD and surrounding the permeable elastic membrane. In certain embodiments, the multilayered tablet core is a bilayered tablet core. In certain embodiments, the gastroretentive composition swells in 60 minutes or less to a size that prevents its passage through the pyloric sphincter, provides a floating lag time of less than 45 minutes, remains in the swollen state for about 8-14 hours, and provides an extended release of CD/LD for a period of about 8-14 hours.

In certain embodiments, the osmotic, controlled, floating gastroretentive CD/LD compositions of the disclosure reduce degradation of LD, and provide steady delivery of CD and LD in the GI tract due to the presence of a swellable water-soluble hydrophilic polymer comprising polyethylene oxide with an average molecular weight of greater than about 600K Da, e.g., POLYOX™ 60 (MW-2M Da) in the push layer, that swells rapidly via imbibition of water from gastric fluid to (1) increase the size of the dosage form to promote gastric retention, (2) osmotically control the release of drug by providing a constant pressure from the push layer on the pull layer comprising the drug dispersion/solution, (3) support the membrane and maintain the integrity of the tablet in a swollen state, and (4) entrap generated gas (e.g., CO₂) to provide buoyancy. In certain embodiments, the gastroretentive CD/LD compositions of the disclosure are provide steady delivery of CD and LD in the GI tract due to the presence at least one polyethylene oxide, having an average molecular weight of about 200K Da, and optionally, a polyethylene oxide having an average molecular weight of greater than or equal to 600K Da, e.g., about 7M Da, in the pull layer. In certain embodiments, the membrane, due to its high elasticity and tensile strength, expands rapidly with an outward pressure on the membrane from the generated CO₂ gas. In certain embodiments, as the dosage form comes in contact with a dissolution medium, the high permeability of the membrane allows for a rapid ingress of the dissolution medium and generation of CO₂, the high elasticity of the membrane allows for rapid expansion of the membrane with the generation of CO₂, followed by swelling of the core to support the membrane and maintain the integrity of the dosage form. In certain embodiments, the tablet core swells and entraps CO₂ to provide buoyancy to the dosage form. In certain embodiments, the swelling of the tablet core is due to the swelling of the pull layer and the push layer.

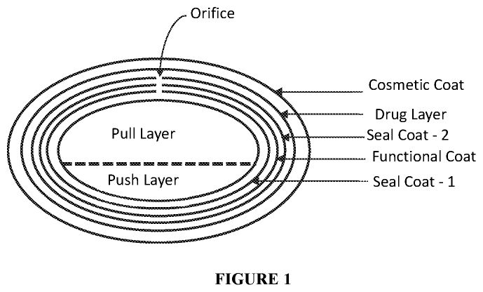

For the purpose of illustration and not limitation, FIG. 1 provides a schematic representation of the gastroretentive dosage form, according to certain embodiments, illustrating a bilayer tablet core, comprising a push layer and a pull layer, Seal Coat-1 surrounding the tablet core, a permeable elastic membrane surrounding Seal Coat-1, Seal Coat-2 surrounding the permeable membrane, an IR drug layer over the Seal Coat-2, a Cosmetic Coat surrounding the IR drug layer, and an orifice passing through Seal Coat-1, the membrane, and Seal Coat-2, wherein the orifice is in fluid communication with the pull layer.

### Swellable Multilayered Tablet Core

In certain embodiments, the swellable multilayered tablet core comprises at least one push layer and at least one pull layer. In certain embodiments, the push layer and the pull layer are compressed into a multilayered tablet core. In certain embodiments, the multilayered tablet core is a horizontally compressed bilayered tablet core. In certain embodiments, horizontal compression of the pull layer and the push enhances tablet buoyancy for gastric retention. In certain embodiments, the multilayered tablet core comprises a push layer between two pull layers. In certain embodiments, wt % ratio of the pull layer and the push layer in the tablet core is between about 1:1 to about 6:1. In certain embodiments, wt % ratio of the pull layer and the push layer in the tablet core is about 1:1, about 1.5:1, about 2:1, about 2.5:1, about 3:1, about 3.5:1, about 4:1, about 4.5:1, about 5:1, about 5.5:1, about 6:1, or any intermediate ratios therein.

### Pull Layer

In certain embodiments, the pull layer includes CD, LD, a swellable water-soluble hydrophilic polymer, an acid, and a gas-generating agent. In certain embodiments, the swellable water-soluble hydrophilic polymer comprises a low viscosity hydroxypropyl methylcellulose, hydroxypropyl cellulose, carbomer, or a polyethylene oxide polymer (POLYOX®). In certain embodiments, the pull layer includes a polyethylene oxide polymer having an average molecular weight of less than about 1M (million) Da. In certain embodiments, the pull layer includes a polyethylene oxide polymer having an average molecular weight of less than or equal to about 1M (million) Da and a polyethylene oxide polymer having an average molecular weight of greater than 1M Da. In certain embodiments, the polyethylene oxide polymer with an average molecular weight of greater than about 1M Da and the polyethylene oxide polymer with an average molecular weight of less than or equal to about 1M Da are present in a weight ratio of between 1:99 and 10:90. In certain embodiments, the polyethylene oxide polymer with an average molecular weight of greater than about 1M Da and the polyethylene oxide polymer with an average molecular weight of less than or equal to about 1M Da are present in a weight ratio of between 1:99, about 2:98, about 3:97, about 4:96, about 5:95, about 6:94, about 7:93, about 8:92, about 9:91, or about 10:90.

In certain embodiments, the pull layer includes a polyethylene oxide polymer having an average molecular weight of 200K Da (POLYOX® N 80) and a polyethylene oxide polymer having an average molecular weight of about 7M Da (POLYOX® 303). In certain embodiments, the polyethylene oxide polymer with an average molecular weight of about 7M Da and the polyethylene oxide polymer with an average molecular weight of about 200K Da are present in a weight ratio of between 1:99 and 10:90. In certain embodiments, the polyethylene oxide polymer with an average molecular weight of about 7M Da and the polyethylene oxide polymer with an average molecular weight of about 200K Da are present in a weight ratio of between 1:99, about 2:98, about 3:97, about 4:96, about 5:95, about 6:94, about 7:93, about 8:92, about 9:91, or about 10:90.

In certain embodiments, the pull layer includes at least one polyethylene oxide polymer with an average molecular weight of about 100K, about 200K, about 300K, about 400K, about 500K, about 600K, about 700K, about 800K, about 900K, about 1M Da, or intermediate values therein; and at least one polyethylene oxide polymer with an average molecular weight of about 2M Da, about 4M Da, about 5M Da, about 7M Da, or any intermediate values therein. In certain embodiments, the pull layer further includes a binder, a stabilizer to prevent the degradation of the polyethylene oxide polymer, and a disintegrant. In certain embodiments, the presence of disintegrant is optional. In certain embodiments, the pull layer includes intermediate drug granules comprising CD and LD (CD/LD co-granulates). In certain embodiments, the CD/LD co-granulates are mixed with an extragranular component to provide a pull layer blend. In certain embodiments, CD/LD co-granulates are made via dry granulation or wet granulation. In certain embodiments, the CD/LD co-granulates are made by wet granulation. In certain embodiments, the solvent used in wet granulation process comprises ethanol 200 proof, isopropyl alcohol (99% v/v), water, or a mixture thereof. In certain embodiments, solvents used in the wet granulation process are substantially free of water. In certain embodiments, the CD/LD co-granulates comprise CD, LD, a polyethylene oxide polymer having an average molecular weight of less than or equal to about 1M (million) Da, a polyethylene oxide polymer having an average molecular weight of greater than 1M Da, an acid, a binder, a stabilizer, and optionally, a disintegrant. In certain embodiments, the extragranular components comprise at least one gas-generating agent. In certain embodiments, the gas-generating agent(s) is present in the CD/LD co-granulates and/or the extragranular component. In certain embodiments, the extragranular component can further include a filler, a glidant, and/or a lubricant. In certain embodiments, the pull layer includes at least one acid to accelerate generation of CO₂ from the gas-generating agents and/or stabilize CD. In certain embodiments, the acid is micronized to accelerate the generation of CO₂ for rapid expansion and floatation of the dosage form; and enhance the stability of CD. In certain embodiments, the acid is present in CD/LD co-granulates and/or the extragranular component.

In certain embodiments, the pull layer includes polyethylene oxide polymer as a binder and/or a suspending agent. In certain embodiments, the pull layer includes a polyethylene oxide polymer as a release controlling agent. In certain embodiments, average molecular weight of the polyethylene oxide polymer in the pull layer affects CD/LD drug release from the dosage form, e.g., an increase in the average molecular weight of the polyethylene oxide polymer increases viscosity of the pull layer and increases control on the drug release. In certain embodiments, the viscosity of the pull layer can be adjusted to provide a desired steady drug release profile. In certain embodiments, the viscosity of the pull layer can be modified by mixing a small amount of a polyethylene oxide polyethylene oxide polymer with an average molecular weight of greater than about 1M, e.g., POLYOX® 303, with at least one polyethylene oxide polymer with an average molecular weight of less than or equal to about 1M Da, e.g., POLYOX® N80. In certain embodiments, the pull layer includes a polyethylene oxide polymer with an average molecular weight of about 100K, 200K, 300K, 400K, 500K, 600K Da, 600K Da, 800K Da, 900K Da, 1M Da, or any intermediate values therein, and a polyethylene oxide polymer with an average molecular weight of about 2M Da, about 4M da, about 5M Da, or about 7M Da. In certain embodiments, the pull layer includes at least one polyethylene oxide polymer with an average molecular weight of about 200K Da and at least one polyethylene oxide polymer with an average molecular weight of about 2M, about 4M, about 5M, or about 7M Da. In certain embodiments, the pull layer includes (1) a polyethylene oxide polymer with an average molecular weight of greater than about 1M Da and (2) a polyethylene oxide polymer with an average molecular weight of less than or equal to about 1M Da in a ratio of between about 1:99 and about 10:90. In certain embodiments, the total amount of the polyethylene oxide polymer in the pull layer ranges from about 5 wt % to about 80 wt %, from about 10 wt % to about 75 wt %, from about 15% to about 70 wt %, from about 20 wt % to about 65 wt %, from about 25 wt % to about 60 wt %, from about 30 wt % to about 55 wt %, from about 35 wt % to about 50 wt %, about 30 wt %, about 25 wt %, about 20 wt %, about 15 wt %, about 10 wt %, about 5 wt %, or any intermediate values therein, based on the total weight of the pull layer.

In certain embodiments, the pull layer includes binders selected from the group consisting of, but not limited to, povidone K 90, hypromellose, starch, acacia, gellan gum, low viscosity hydroxypropyl cellulose (viscosity of between 75-150 cp in a 5% w/w aqueous solution), methylcellulose, sodium methylcellulose, polyvinyl alcohol, polyvinyl acetates (e.g., KOLLICOAT® SR), polyethylene oxide, polyethylene glycol, alginates, pegylated polyvinyl alcohol, and any combination thereof. In certain embodiments, the binder is a low viscosity hydroxypropyl cellulose.

In certain embodiments, binders are present in an amount of from about 0.5 wt % to about 20 wt %, based on the total weight of the pull layer. In certain embodiments, the binders are present in an amount of about 0.5 wt %, about 0.6 wt %, about 0.7 wt %, about 0.8 wt %, about 0.9 wt %, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, or any intermediates values therein, based on the total weight of the pull layer.

In certain embodiments, the pull layer includes at least one stabilizer to prevent or reduce degradation of the polyethylene oxide polymer. In certain embodiments, the stabilizer is an antioxidant selected from the group consisting of, but not limited to, ascorbic acid and its salts, α-tocopherol, sulfite salts such as sodium metabisulfite or sodium sulfite, sodium sulfide, butylated hydroxyanisole (BHA), butylated hydroxytoluene (BHT), ascorbyl palmitate, propyl gallate, and any combination thereof. In certain embodiments, the antioxidant is α-tocopherol. In certain embodiments, the stabilizer is present in an amount of from about 0.01 wt % to about 20 wt %, based on the total weight of the pull layer. In certain embodiments, the stabilizer is present in an amount of about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.10 wt %, about 0.2 wt %, about 0.3 wt %, about 0.4 wt %, about 0.5 wt %, about 1 wt %, about 5 wt %, about 10 wt %, about 15 wt %, about 20 wt %, or any intermediate values therein, based on the total weight of the pull layer.

In certain embodiments, the pull layer includes at least one acid selected from the group consisting of succinic acid, citric acid, malic acid, fumaric acid, stearic acid, tartaric acid, boric acid, benzoic acid, and combinations thereof. In certain embodiments, the acid is succinic acid. In certain embodiments, the acid is present in an amount of from about 5 wt % to about 50 wt %, based on the total weight of the pull layer. In certain embodiments, the acid is present in an amount of about 5 wt %, about 10 wt %, about 15 wt %, about 20 wt %, about 25 wt %, about 30 wt %, about 35 wt %, about 40 wt %, about 45 wt %, about 50 wt %, or any intermediate values therein, based on the total weight of the pull layer. In certain embodiments, generation of CO₂ from the gas-generating agents depends upon the particle size of the acid, e.g., smaller particle size provides faster generation of CO₂. In certain embodiments, presence of succinic acid in pull layer stabilizes CD that reduces degradation of LD. In certain embodiments, particle size of succinic acid affects stability of CD and LD. In certain embodiments, the D90 particle size of succinic acid is between about 10 microns and about 150 microns.

In certain embodiments, the pull layer includes at least one gas-generating agent for rapid expansion and floatation of the dosage form. The gas-generating agent generates CO₂ with imbibition of gastric fluid in the dosage form. In certain embodiments, the presence of acid in the pull layer results in faster generation of CO₂ with imbibition of gastric fluid in the dosage form. Examples of gas-generating agents present in the pull layer include, but are not limited to, all organic and inorganic carbonates, e.g., carbonate and bicarbonate salts of alkali and alkaline earth metals, that can interact with acid for in situ gas generation. In certain embodiments, the gas-generating agent is sodium bicarbonate, sodium carbonate, magnesium carbonate, and/or calcium carbonate. In certain embodiments, a mixture of calcium carbonate and sodium bicarbonate provides desired sustained release of CO₂. In certain embodiments, the gas-generating agent is present in an amount of from at least about 5 wt % to about 50 wt % of the pull layer weight. In certain embodiments, the gas-generating agent is present in an amount of about 5 wt %, about 10 wt %, about 15 wt %, about 20 wt %, about 25 wt %, about 30 wt %, about 35 wt %, about 40 wt %, about 45 wt %, about 50 wt %, or any intermediate values therein, based on the total weight of the pull layer.

In certain embodiments, the gas generating agent comprises a mixture of sodium bicarbonate and calcium carbonate. In certain embodiments, the pull layer comprises a mixture of sodium bicarbonate and calcium carbonate as gas generating agent, and an acid comprising succinic acid that interacts with the gas generating agent to generate CO₂. In certain embodiments, the pull layer comprises equinormal amounts of acid and gas-generating agent (e.g., a mixture of calcium carbonate and sodium bicarbonate).

In certain embodiments, the pull layer can comprise a disintegrant including carmellose calcium, carboxymethylstarch sodium, croscarmellose sodium, crospovidone (crosslinked homopolymer of N-vinyl-2-pyrrolidone), low-substituted hydroxypropyl celluloses, sodium starch glycolate, colloidal silicon dioxide, alginic acid and alginates, acrylic acid derivatives, and various starches, or any combinations thereof.

In certain embodiments, the pull layer includes at least one lubricant selected from the group comprising magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid, and any combinations thereof. In certain embodiments, the lubricant is magnesium stearate. In certain embodiments, the lubricant is present in an amount of from about 0.5 wt % to about 5 wt %, based on the total weight of the pull layer. In certain embodiments, the lubricant is present in an amount of about 0.5 wt %, about 0.6 wt %, about 0.7 wt %, about 0.8 wt %, about 0.9 wt %, about 1.0 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2.0 wt %, about 2.5 wt %, about 3.0 wt %, about 3.5 wt %, about 4.0 wt %, about 5.0 wt %, or any intermediate values therein, based on the total weight of the pull layer.

In certain embodiments, the pull layer includes at least one glidant selected from the group comprising talc, colloidal silicon dioxide, magnesium trisilicate, powdered cellulose, starch, tribasic calcium phosphate, and any combination thereof. In certain embodiments, the glidant is colloidal silicon dioxide. In certain embodiments, the glidant is present in an amount of from about 0.1 wt % to about 5 wt %, based on the total weight of the pull layer. In certain embodiments, the glidant is present in an amount of about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 2 wt %, about 3 wt %, about 4 wt %, about 5 wt %, or any intermediate valued therein, based on the total weight of the pull layer.

In certain embodiments, the pull layer further comprises mannitol. In certain embodiments, mannitol is used as a filler and/or as a compression aid. In certain embodiments, mannitol is used as a secondary osmotic agent. In certain embodiments, mannitol is present in an amount of from about 1 wt % to about 20 wt % of the pull layer.

In certain embodiments, the pull layer includes multiple layers containing CD and LD to provide drug release with increasing drug concentration.

### Push Layer

In certain embodiments, the push layer includes a swellable water-soluble hydrophilic polymer, an osmogen, a lubricant, and a color pigment. In certain embodiments, the swellable water-soluble hydrophilic polymer is polyethylene oxide polymer. In certain embodiments, the polyethylene oxide polymer in the push layer has an average molecular weight of greater than about 600K Da. In certain embodiments, average molecular weight of the polyethylene oxide polymer in the push layer is about 600K, about 700K, about 800K, about 900K, about 1M, about 2M, about 3M, about 4M, about 5M, about 6M, about 7M Da, or any intermediate values thereof. In certain embodiments, the amount of polyethylene oxide polymer in the push layer is sufficient to provide substantially complete recovery of CD and LD, (i.e., the pull layer is substantially expelled); the remaining dosage form, with push layer only, collapses/shrinks for complete emptying of the composition from the GI tract and the patient. In certain embodiments, the polyethylene oxide polymer is present in an amount of from about 50 wt % to about 95 wt %, based on the total weight of the push layer. In certain embodiments, the polyethylene oxide polymer is present in an amount of about 50 wt %, about 55 wt %, about 60 wt %, about 65 wt %, about 70 wt %, about 75 wt %, about 80 wt %, about 85 wt %, about 90 wt %, about 95 wt %, or any intermediate values therein, based on the total weight of the push layer. In certain embodiments, the polyethylene oxide polymer in the push layer is present in an amount of amount 10 wt % to about 30 wt %, based on the total weight of the coated tablet composition. In certain embodiments, the polyethylene oxide polymer is present in an amount of about 11 wt %, about 12 wt %, about 13 wt %, about 14 wt %, about 15 wt %, about 16 wt %, about 17 wt %, about 18 wt %, about 19 wt %, about 20 wt %, about 25 w %, about 30 wt %, or any intermediate values therein, based on the total weight of the coated tablet composition.

In certain embodiments, the amount and the average molecular weight of polyethylene oxide in the push layer affects the drug release profile. In certain embodiments, the average molecular weight of polyethylene oxide in the push layer is selected to provide substantial expansion of the push layer for substantially complete drug recovery at a desired time period. In certain embodiments, the average molecular weight of polyethylene oxide in the push layer provides substantially complete drug recovery, while keeping the dosage form intact.

In certain embodiments, the push layer includes a lubricant selected from the group comprising magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid, and any combinations thereof. In certain embodiments, the lubricant is magnesium stearate. In certain embodiments, the lubricant is present in an amount of about 0.5 wt % to about 2 wt %, based on the total weight of the push layer. In certain embodiments, the lubricant is present in an amount of about 0.5 wt %, about 0.6 wt %, about 0.7 wt %, about 0.8 wt %, about 0.9 wt %, about 1.0 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2.0 wt %, or any intermediate values therein, based on the total weight of the push layer.

In certain embodiments, the push layer comprises at least one osmogen. In certain embodiments, the osmogen includes ionic compounds of inorganic salts that provide a concentration differential for osmotic flow of liquid into the composition. The rate at which the water-soluble polymer in the push layer absorbs water depends on the osmotic pressure generated by the push layer and the permeability of the membrane coating. As the water-soluble polymer in the push layer absorbs water, it expands in volume, which pushes the drug solution/suspension/or dispersion present in the pull layer out of the tablet core through the orifice in the membrane. In certain embodiments, the generation of CO₂ from the gas generating agents and the acid present in the dosage form can result in excess pressure buildup within the membrane and the presence of orifice in the membrane releases this excess pressure buildup. Such release of the excess pressure buildup prevents membrane tearing and keeps the dosage form intact. In certain embodiments, the orifice releases excess pressure buildup during swelling of the dosage form, e.g., due to the push layer, and allows the membrane to remain intact under hydrodynamic conditions of the GI tract. In certain embodiments, the osmogen is an ionic compound selected from the group consisting of sodium chloride, potassium chloride, potassium sulfate, lithium sulfate, sodium sulfate, lactose and sucrose combination, lactose and dextrose combination, sucrose, dextrose, mannitol, dibasic sodium phosphate, and combinations thereof. In certain embodiments, the osmogen is sodium chloride. In certain embodiments, the osmogen is present in an amount of from about 5 wt % to about 30 wt %, based on the total weight of the push layer. In certain embodiments, the osmogen is present in an amount of about 5 wt %, about 6 wt %, about 7 wt %, about 8 wt %, about 9 wt %, about 10 wt %, about 15 wt %, about 20 wt %, about 25 wt %, about 30 wt %, or any intermediate values therein, based on the total weight of the push layer.

In certain embodiments, the push layer includes at least one pigment for identifying the push layer in the multilayered tablet core. In certain embodiments, the pigment in the push layer is useful for identifying the push-layer side while drilling a delivery orifice on the drug-layer side (pull layer side) of the coated multilayered tablets. In certain embodiments, the push layer includes at least one pigment comprising iron oxide or lake-based colors. In certain embodiments, the pigment is a lake-based color. In certain embodiments, the pigment is an iron oxide pigment, e.g., oxide pigment black or Red blend. In certain embodiments, the pigment is present in an amount of from about 0.5 wt % to about 2 wt %, based on the total weight of the push layer.

### Membrane/Functional Coat

The compositions of the disclosure comprise a membrane that is a water-insoluble, permeable elastic membrane surrounding the multilayer tablet core. The membrane allows the flow of gastric fluid into the composition to initiate gas generation from the gas-generating agents present in the pull layer, and the membrane flexibility allows for an initial rapid expansion and floatation of the composition from the generated gas (e.g., CO₂). In certain embodiments, the membrane comprises at least one water-insoluble permeable polyacrylate/polymethacrylate copolymer (copolymers of ethyl acrylate, methyl methacrylate and trimethylammonioethyl methacrylate chloride) that has a Tg (glass transition temperatures) of between about 50° C. and about 70° C. (e.g., EUDRAGIT® RL PO, EUDRAGIT® RS PO EUDRAGIT® RL 30D, and EUDRAGIT® RS 30D). In certain embodiments, the membrane comprises at least one copolymer of ethyl acrylate, methyl methacrylate, and trimethylammonioethyl methacrylate chloride that has a Tg of between about 60° C. and about 70° C. (e.g., EUDRAGIT® RL PO and EUDRAGIT® RS PO). In certain embodiments, the membrane comprises at least one copolymer of ethyl acrylate, methyl methacrylate, and trimethylammonioethyl methacrylate chloride (1:2:0.2) that has a Tg of between about 50° C. and about 70° C. (e.g., EUDRAGIT® RL PO and EUDRAGIT® RL 30D). In certain embodiments, the membrane comprises at least one copolymer of ethyl acrylate, methyl methacrylate, and trimethylammonioethyl methacrylate chloride (1:2:0.2) that has a Tg of between about 60° C. and about 70° C. (EUDRAGIT® RL PO).

In certain embodiments, the membrane comprises a plasticizer and at least one copolymer of ethyl acrylate, methyl methacrylate, and trimethylammonioethyl methacrylate chloride (1:2:0.2) that has a Tg of about 63° C. (EUDRAGI® RL PO). EUDRAGI® RL PO copolymer provides a highly permeable elastic membrane due to its uniquely high permeability and a favorable Tg of about 63° C. In certain embodiments, the membrane further includes a plasticizer in an amount that can substantially enhance the membrane elasticity for rapid expansion of the membrane with the generation of gas from the gas generating agent and the acid. In certain embodiments, the plasticizer is present in an amount of about 10-25% w/w, based on the total weight of the EUDRAGIT® RL PO copolymer. The plasticizers enhance membrane elasticity, ensuring that the membrane does not rupture upon expanding and that the osmotic gastroretentive drug delivery system provides the desired characteristics for drug release, hydrodynamic balance, and mechanical strength to withstand variations in pH and shear in the stomach during either fed or fasted conditions. In certain embodiments, as dissolution of the active agent in the tablet core proceeds, the plasticizer leaches out of the membrane. In certain embodiments, regardless of plasticizer leaching, the membrane retains enough elasticity to keep the dosage form intact until at least 75%, e.g., about 80%, of the drug, based on the total weight of the drug present in the dosage form, is released. In certain embodiments, regardless of plasticizer leaching, the membrane is sufficiently elasticity to squeeze the dosage form out of the stomach through the pyloric sphincter after about 80% w/w, based on the total weight of the drug, is released from the dosage form. In certain embodiments, the membrane includes a hydrophilic or a lipophilic plasticizer. Hydrophilic plasticizers suitable for the disclosure include, but are not limited to, glycerin, polyethylene glycols, polyethylene glycol monomethyl ether, propylene glycol, and sorbitol sorbitan solution. Lipophilic plasticizers suitable for the disclosure include, but are not limited to, acetyl tributyl citrate, acetyl triethyl citrate, castor oil, diacetylated monoglycerides, dibutyl sebacate, diethyl phthalate, triacetin, tributyl citrate, triethyl citrate, gelucire 39/01, and gelucire 43/01. In certain embodiments, the plasticizers comprise various polyethylene glycols, glycerin, and/or triethyl citrate. In a preferred embodiment of the disclosure, the plasticizer is triethyl citrate.

In certain embodiments, the membrane comprises a water-insoluble polymer, a plasticizer, and at least one pore former comprising a water-soluble nonionic polymer. In certain embodiments, the pore formers and plasticizers modify membrane permeability, membrane elasticity, and tensile strength. In certain embodiments, the membrane does not include any pore former. In certain embodiments, examples of water-insoluble permeable components of the permeable elastic membrane include, but are not limited to, copolymers of ethyl acrylate, methyl methacrylate, and trimethylammonioethyl methacrylate chloride (e.g., EUDRAGIT® RL 30D, EUDRAGIT® RS 30D, EUDRAGIT® RL PO or EUDRAGIT® RS PO).

In certain embodiments, the membrane further includes an anti-tacking agent selected from the group comprising talc, colloidal silicon dioxide, magnesium trisilicate, powdered cellulose, starch, and tribasic calcium phosphate. In certain embodiments, the anti-tacking agent is colloidal silicon dioxide.

In certain embodiments, strength of the membrane depends upon compatibility/homogeneity of the water-insoluble polymers present in the coating composition. In certain embodiments, the tablet core is coated with a coating composition comprising at least one copolymer of ethyl acrylate, methyl methacrylate, and trimethylammonioethyl methacrylate chloride that has a Tg of between about 60° C. and about 70° C., e.g., EUDRAGIT® RL PO and/or EUDRAGIT® RS PO, a plasticizer, and an anti-tacking agent in a suitable solvent. In certain embodiments, the solvent used for coating comprises acetone, water, ethanol, isopropyl alcohol, or a mixture thereof. In certain embodiments, the solvent is a mixture of acetone and water, a mixture of ethanol and water, a mixture of ethanol and isopropyl alcohol, a mixture of isopropyl alcohol and water, or a mixture of water, ethanol, and isopropyl alcohol. In certain embodiments, the solvent is a mixture of acetone and water. In certain embodiments, the ratio of the solvent and water ranges from about 80:20 to about 99:1. In certain embodiments, the ratio of acetone and water is about 80:20, about 85:15, about 90:10, and about 95:5.

In certain embodiments, the coating composition includes at least one of EUDRAGIT® RL PO or EUDRAGIT® RS PO to improve permeability, and at least one plasticizer to improve mechanical strength (tensile strength). In certain embodiments, the coating composition is prepared using powder forms of EUDRAGIT®, e.g., EUDRAGIT® RL PO or EUDRAGIT® RS PO, instead of EUDRAGIT® dispersions, e.g., EUDRAGIT® RS 30D or EUDRAGIT® RL 30D. It was unexpectedly observed that gastroretentive compositions coated with a coating composition comprising EUDRAGIT® RL PO copolymer provided superior gastroretentive attributes compared to gastroretentive compositions coated with coating compositions comprising EUDRAGIT® RL 30D (notwithstanding similar permeabilities of the two copolymers). It was further unexpectedly observed that gastroretentive compositions coated with a coating composition comprising EUDRAGIT® RL PO copolymer provided superior gastroretentive attributes compared to gastroretentive compositions coated with coating compositions comprising EUDRAGIT® RS PO (notwithstanding similar Tg of the two copolymers). In certain embodiments, the gastroretentive dosage forms of the disclosure containing permeable elastic membranes comprising EUDRAGIT® RL PO and a plasticizer, provided superior gastroretentive attributes, e.g., short floating lag time, rapid volume expansion, and sustained drug release for extended periods.

In certain embodiments, permeability, elasticity, and tensile strength of the membrane determines the floating time and floating lag time of the osmotic gastroretentive delivery system of the disclosure. In certain embodiments, the membrane permeability, elasticity, and tensile strength is based on permeability and elasticity of the polymers present in the membrane. In certain embodiments, the compositions of the disclosure exhibit increase in floating time and decrease in floating lag time with increasing membrane permeability. In certain embodiments, permeability of the copolymer of ethyl acrylate, methyl methacrylate, and trimethylammonioethyl methacrylate chloride is enhanced on exchange of chloride anion with other anions. In certain embodiments, the chloride anion is exchanged with nitrate ions, sulfate ions, succinate ions, or acetate ions. In certain embodiments, exchange of chloride anions with anions of higher hydrated anion radius improves membrane permeability.

In certain embodiments, permeability of the permeable elastic membrane is adjusted to provide a floating lag time of less than about 45 minutes and floating time of from about 8 hours to about 14 hours. In certain embodiments, the self-regulating, osmotic, floating gastroretentive dosage form of the disclosure containing membranes comprising EUDRAGIT® RL PO and/or EUDRAGIT® RS PO, exhibit a floating lag time of less than about 45 minutes and floating time of from about 8 hours to about 14 hours.

In certain embodiments, the EUDRAGIT® RL PO and/or EUDRAGIT® RS PO are present in an amount of between about 70% and about 90% w/w, based on the total weight of the membrane composition, to provide desired tensile strength, and elasticity for rapid expansion of the membrane. In certain embodiments, plasticizer is present in an amount of between about 10 wt % and about 25 wt %, between about 10 wt % and about 20 wt %, between about 10 wt % and about 15 wt %, and any intermediate ranges there in, based on the total weight of EUDRAGIT® RL PO and/or EUDRAGIT® RS PO, to provide desired tensile strength, and elasticity for rapid expansion of the membrane. In certain embodiments, the plasticizer is present in an amount of at least about 10 wt %, at least about 11 wt %, at least about 12 wt %, at least about 13 wt %, at least about 14 wt %, at least about 15 wt %, at least about 16 wt %, at least about 17 wt %, at least about 18 wt %, at least about 19 wt %, at least about 20 wt %, at least about 21 wt %, at least about 22 wt %, at least about 23 wt %, at least about 24 wt %, and at least about 25 wt %, based on the total weight of EUDRAGIT® RL PO and/or EUDRAGIT® RS PO.

In certain embodiments, the self-regulating, osmotic, floating gastroretentive dosage form of the disclosure containing membranes comprising EUDRAGIT® RL PO and/or EUDRAGIT® RL 30D, exhibit a floating lag time of less than about 45 minutes and floating time of from about 8 hours to about 14 hours.

In certain embodiments, EUDRAGIT® RL PO and/or EUDRAGIT® RL 30D are present in an amount of between about 70% and about 90% w/w, based on the total weight of the membrane composition, to provide desired tensile strength, and elasticity for rapid expansion of the membrane. In certain embodiments, plasticizer is present in an amount of between about 10 wt % and about 25 wt %, between about 10 wt % and about 20 wt %, between about 10 wt % and about 15 wt %, and any intermediate ranges there in, based on the total weight EUDRAGIT® RL PO and/or EUDRAGIT® RL 30D, to provide desired tensile strength, and elasticity for rapid expansion of the membrane. In certain embodiments, the plasticizer is present in an amount of at least about 10 wt %, at least about 11 wt %, at least about 12 wt %, at least about 13 wt %, at least about 14 wt %, at least about 15 wt %, at least about 16 wt %, at least about 17 wt %, at least about 18 wt %, at least about 19 wt %, at least about 20 wt %, at least about 21 wt %, at least about 22 wt %, at least about 23 wt %, at least about 24 wt %, and at least about 25 wt %, based on the total weight of EUDRAGIT® RL PO and/or EUDRAGIT® RL 30D.

In certain embodiments, the permeable elastic membrane comprises EUDRAGIT® RL PO, a plasticizer, and talc. In certain embodiments, the EUDRAGIT® RL PO is present in an amount of between about 70% and about 90% w/w, based on the total weight of the membrane composition, to provide desired tensile strength, and elasticity for rapid expansion of the membrane. In certain embodiments, plasticizer is present in an amount of between about 10 wt % and about 25 wt %, between about 10 wt % and about 20 wt %, between about 10 wt % and about 15 wt %, and any intermediate ranges there in, based on the total weight of the EUDRAGIT® RL PO, to provide desired tensile strength, and elasticity for rapid expansion of the membrane. In certain embodiments, the plasticizer is present in an amount of at least about 10 wt %, at least about 11 wt %, at least about 12 wt %, at least about 13 wt %, at least about 14 wt %, at least about 15 wt %, at least about 16 wt %, at least about 17 wt %, at least about 18 wt %, at least about 19 wt %, at least about 20 wt %, at least about 21 wt %, at least about 22 wt %, at least about 23 wt %, at least about 24 wt %, and at least about 25 wt %, based on the total weight of the EUDRAGIT® RL PO.

In certain embodiments, the anti-tacking agent is present in an amount of from about 5 wt % to about 30 wt %, based on the total weight of the copolymer, e.g., EUDRAGIT® RL PO and/or EUDRAGIT® RL 30D. In certain embodiments, the anti-tacking agent is present in an amount of about 5 wt %, about 10 wt %, about 15 wt %, about 20 wt %, about 25 wt %, about 30 wt %, or any intermediate values therein, based on the total weight of the of EUDRAGIT® RL PO and/or EUDRAGIT® RL 30D.

In certain embodiments, the anti-tacking agent is present in an amount of from about 5 wt % to about 30 wt %, based on the total weight of the copolymer, e.g., EUDRAGIT® RL PO and/or EUDRAGIT® RS PO. In certain embodiments, the anti-tacking agent is present in an amount of about 5 wt %, about 10 wt %, about 15 wt %, about 20 wt %, about 25 wt %, about 30 wt %, or any intermediate values therein, based on the total weight of the of EUDRAGIT® RL PO and/or EUDRAGIT® RS PO.

In certain embodiments, the anti-tacking agent is present in an amount of from about 5 wt % to about 30 wt %, based on the total weight of the copolymer, e.g., EUDRAGIT® RL PO. In certain embodiments, the anti-tacking agent is present in an amount of about 5 wt %, about 10 wt %, about 15 wt %, about 20 wt %, about 25 wt %, about 30 wt %, or any intermediate values therein, based on the total weight of EUDRAGIT® RL PO.

In certain embodiments, the membrane includes a delivery orifice in fluid communication with the pull layer. In certain embodiments, the gastroretentive compositions of the disclosure containing a membrane comprising EUDRAGIT® RS PO, release drug primarily through the orifice. In certain embodiments, the drug is released through the orifice as a dispersion/suspension, at a desired release rate, based on the average molecular weight of polyethylene oxide in the push and the pull layer. In certain embodiments, swelling rate of polyethylene oxide in the push layer depends upon the amount of osmogen, and average molecular weight of polyethylene oxide present in the push layer. In certain embodiments, the size of the orifice in the membrane and average molecular weight of polyethylene oxide in the pull layer controls the release of the CD and LD from the dosage form. In certain embodiments, the gastroretentive compositions of the disclosure containing a membrane comprising EUDRAGIT® RL PO, release drug primarily through the membrane diffusion. In certain embodiments, size of orifice does not affect drug release rate for the gastroretentive compositions of the disclosure containing a membrane comprising EUDRAGIT® RL PO.

### Immediate Release Drug Layer

In certain embodiments, the self-regulating, oral, osmotic, floating gastroretentive compositions of the disclosure provide a biphasic drug release comprising an immediate release and an extended release of same drugs, e.g., CD and LD. In certain embodiments, the gastroretentive CD/LD compositions providing a biphasic drug release contain one or more immediate release drug layers over the permeable elastic membrane containing an orifice. In certain embodiments, the immediate release drug layer comprises CD and LD for immediate release, a film-forming polymer and, optionally, other excipients known in the art. In certain embodiments, the IR drug layer further includes at least one acid to stabilize CD. In certain embodiments, the immediate release drug layer is further coated with an additional layer, e.g., an outermost coat comprising a powder or a film that prevents adherence of the dosage form to itself. In certain embodiments, the gastroretentive CD/LD compositions of the disclosure containing an IR drug layer further contain a Cosmetic Coat/Over Coat. In certain embodiments, the IR drug layer is present immediately below the Cosmetic Coat/Over Coat. In certain embodiments, a Cosmetic Coat/Over Coat surrounds the permeable or semipermeable membrane or the immediate release drug layer. In certain embodiments, the immediate release drug layer is surrounded by Seal Coat-2, a Cosmetic Coat/Over Coat over Seal Coat-2, and a Final Coat/Clear Coat over the Cosmetic Coat, wherein the Final Coat/Clear coat is the outermost layer. In certain embodiments, the immediate release drug layer is surrounded by Seal Coat-2, and a Cosmetic Coat/Over Coat, wherein the Cosmetic Coat/Over Coat is the outermost layer.

In certain embodiments, the IR drug layer contains CD and LD in a combined weight of between about 70 wt % and about 90 wt %, based on the total weight of the IR drug layer. In certain embodiments, the IR drug layer contains about 70 wt %, about 75 wt %, about 80 wt %, about 85 wt %, about 90 wt %, or any intermediate values therein of the combined weight of CD and LD, based on the total weight of the IR drug layer.

Examples of soluble film-forming polymers that can be used in the immediate release drug layer include, but are not limited to, soluble cellulose derivatives, e.g., methyl cellulose; hydroxypropyl cellulose; hydroxyethyl cellulose; hypromellose; various grades of povidone; polyvinyl alcohol and its derivatives, e.g., KOLLICOAT® IR; soluble gums; and others. In certain embodiments, the film forming polymer is a low viscosity hydroxypropyl cellulose (HPC). In certain embodiments, the HPC is present in an amount of about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, or any intermediate values therein, based on the total weight of the IR drug layer.

In certain embodiments, the IR drug layer further comprises antioxidants, surface-active agents, plasticizers and humectants, such as PEGs, various grades of polysorbates, and sodium lauryl sulfate. In certain embodiments, the IR drug layer includes at least one stabilizer to prevent degradation of CD. In certain embodiments, the stabilizer is an antioxidant selected from the group consisting of, but not limited to, ascorbic acid and its salts, α-tocopherol, sulfite salts such as sodium metabisulfite or sodium sulfite, sodium sulfide, butylated hydroxyanisole (BHA), butylated hydroxytoluene (BHT), ascorbyl palmitate, propyl gallate, and any combination thereof. In certain embodiments, the antioxidant is α-tocopherol. In certain embodiments, the stabilizer is present in an amount of from about 0.01 wt % to about 5 wt %, based on the total weight of the drug layer. In certain embodiments, the stabilizer is present in an amount of about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.10 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 2 wt %, about 3 wt %, about 4 wt %, about 5 wt %, or any intermediate values therein, based on the total weight of the IR drug layer.

In certain embodiments, the IR drug layer includes at least one acid selected from the group consisting of succinic acid, citric acid, malic acid, fumaric acid, stearic acid, tartaric acid, boric acid, benzoic acid, and combinations thereof. In certain embodiments, the acid is succinic acid. In certain embodiments, the acid is present in an amount of from about 0.5 wt % to about 10 wt %, based on the total weight of the IR drug layer. In certain embodiments, the acid is present in an amount of about 0.5 wt %, about 1 wt %, about 1.5 wt %, about 2 wt %, about 2.5 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, about 10 wt %, or any intermediate values therein, based on the total weight of the IR drug layer.

### Seal Coat(s), Over Coat/Cosmetic Coat, and Final Coat/Clear Coat

In certain embodiments, the permeable elastic membrane is coated with an Cosmetic Coat/Over Coat comprising OPADRY® II, Pink (mixture of titanium dioxide, talc, guar gum, partially hydrolyzed poly vinyl alcohol, maltodextrin, HPMC, medium chain glyceride, iron oxide red, and iron oxide blue), OPADRY® II, green (mixture of titanium dioxide, talc, guar gum, partially hydrolyzed polyvinyl alcohol, maltodextrin, HPMC, medium chain glyceride, FD & C Blue/Brilliant Blue, Aluminum Lake, and FD & C Yellow/Tartrazine Aluminum lake, Aluminum Lake), or OPADRY® II, Blue (mixture of titanium dioxide, talc, guar gum, partially hydrolyzed polyvinyl alcohol, maltodextrin, HPMC, medium chain glyceride, FD & C Blue/Indigo Carmine Aluminum Lake blue). In certain embodiments, the Over Coat/Cosmetic Coat makes the tablet look smaller than its actual size. In certain embodiments, the Over Coat is surrounded by a Final Coat comprising OPADRY® EZ clear (mixture of talc, guar gum, maltodextrin, HPMC, and medium chain glyceride). In certain embodiments, the Final Coat helps in easy swallowing of the tablets, especially in pediatric and geriatric populations. In certain embodiments, the Over Coat/Cosmetic Coat makes the tablet slippery when in contact with saliva.

In certain embodiments, the composition comprises a seal coat (Seal Coat-1) between the multilayered tablet core and permeable elastic membrane/Functional Coat. In certain embodiments, the composition includes a seal coat (Seal Coat-2) between the permeable elastic membrane and the Over Coat. In certain embodiments, the composition includes a multilayer tablet core coated with a seal coat (Seal Coat-1), a permeable elastic membrane over Seal Coat-1, an additional seal coat (Seal Coat-2) over the permeable elastic membrane, and an Over Coat/Cosmetic Coat over Seal Coat-2. In certain embodiments, the compositions with an IR drug layer further comprise an IR drug layer over Seal Coat-2, Seal Coat-3 over the IR drug layer, and a Cosmetic Coat/Over Coat over Seal Coat-3. In certain embodiments, there is no seal coat between IR drug layer and Cosmetic Coat/Over Coat.

In certain embodiments, the seal coat(s) comprises a pH-independent water-soluble polymer containing a hypromellose (HPMC)-based polymer or a polyvinyl acetate-based polymer. In certain embodiments, the seal coat(s) comprise povidone. In certain embodiments, the seal coat (Seal Coat-1 and Seal Coat-2) comprises a mixture of polyvinyl alcohol, talc, polyethylene glycol, and polysorbate 80 (OPADRY® II, clear). In certain embodiments, Seal Coat-1 is present in an amount of from about 0.5 wt % to about 5 wt % of the uncoated core. In certain embodiments, Seal Coat-1 is present in an amount of about 0.5 wt %, about 1 wt %, about 1.5 wt %, about 2 wt %, about 2.5 wt %, about 3 wt %, about 3.5 wt %, about 4 wt %, about 4.5 wt % about 5 wt %, or any intermediate values therein, based on the total weight of the tablet core without Seal Coat-1. In certain embodiments, Seal Coat-2 is present in an amount of from about 0.1 wt % to about 5 wt %, based on the total weight of the core with of Seal Coat-1 and Functional Coat. In certain embodiments, Seal Coat-2 is present in an amount of about 0.1 wt %, about 0.5 wt %, about 0.3 wt %, about 0.4 wt %, about 0.5 wt %, about 0.6 wt %, about 0.7 wt %, about 0.8 wt %, about 0.9 wt %, about 1 wt %, about 1.5 wt %, about 2 wt %, about 2.5 wt %, about 3 wt %, about 3.5 wt %, about 4 wt %, about 4.5 wt %, about 5 wt %, or any intermediate values therein, based on the total weight of the core with Seal Coat-1 and Functional Coat.

In certain embodiments, the composition includes a multilayer tablet core coated with Seal Coat-1, a permeable elastic membrane/Functional Coat over Seal Coat-1, Seal Coat-2 over the permeable elastic membrane/Functional Coat, and a Cosmetic Coat/Over Coat over Seal Coat-2. In certain embodiments, the compositions with IR layer comprise an IR drug layer over Seal Coat-2, and a Cosmetic Coat/Over Coat over the IR drug layer. In certain embodiments, Seal Coat-3 is present between the IR drug layer and the Cosmetic Coat/Over Coat.

### Gastroretentive Dosage Compositions

In certain embodiments, the gastroretentive dosage forms of the disclosure comprise a multilayered core coated with a permeable membrane containing an orifice. In certain embodiments, the multilayered tablet core comprises a pull layer and a push layer. In certain embodiments, the pull layer can comprise from about 100 mg to about 400 mg, from about 150 mg to about 350 mg, from about 200 mg to about 350 mg, from about 240 mg to about 320 mg, about 200 mg, about 240 mg, about 270 mg, about 315 mg, or about 320 mg of LD. In certain embodiments, the pull layer can further comprise from about 50 mg to about 100 mg, from about 55 mg to about 95 mg, from about 60 mg to about 90 mg, from about 75 mg to about 85 mg, from about 70 mg to about 80 mg, about 55 mg, about 65 mg, about 70 mg, about 75 mg, about 80 mg, or about 85 mg of CD. In certain embodiments, the pull layer can further comprise from about 140 mg to about 200 mg, from about 145 mg to about 195 mg, from about 150 mg to about 190 mg, from about 155 mg to about 185 mg, from about 160 mg to about 180 mg, about 141 mg, about 148 mg, about 190 mg, about 193 mg, about 200 mg of POLYOX™ N80. In certain embodiments, the pull layer can further comprise from about 1 mg to about 10 mg, or about 5 mg of POLYOX™ N303. In certain embodiments, the pull layer can further comprise from about 5 mg to about 10 mg, or about 8 mg of hydroxypropyl cellulose. In certain embodiments, the pull layer can further comprise from about 50 mg to about 125 mg, from about 60 mg to about 100 mg, about 50 mg, about 75 mg, about 100 mg, or about 125 mg of succinic acid. In certain embodiments, the pull layer can further comprise from about 25 mg to about 125 mg, about 50 mg, or about 100 mg of sodium bicarbonate. In certain embodiments, the pull layer can further comprise from about 20 mg to about 150 mg, from about 50 mg to about 100 mg, about 25 mg, about 75 mg, or about 138 mg of calcium carbonate. In certain embodiments, the pull layer can further comprise from about 0.1 mg to about 2 mg, from about 1 mg to about 1.5 mg, about 0.5 mg, or about 2 mg of α-tocopherol. In certain embodiments, the pull layer can further comprise from about 1 mg to about 5 mg, or about 3.5 mg of Cab-O-Sil®. In certain embodiments, the pull layer can further comprise from about 40 mg to about 55 mg, about 44 mg, or about 52 mg of mannitol (PARTECK® M200). In certain embodiments, the pull layer can further comprise from about 1 mg to about 20 mg, from about 10 mg to about 15 mg, about 10 mg, or about 13 mg of magnesium stearate.

In certain embodiments, the push layer can comprise from about 175 mg to about 250 mg, from about 200 mg to about 225 mg, about 197 mg, about 218 mg, about 219 mg, about 220 mg, or about 221 mg of POLYOX™ N60. In certain embodiments, the push layer can further comprise from about 20 mg to about 30 mg, about 22 mg, or about 25 mg of sodium chloride. In certain embodiments, the push layer can further comprise from about 1 mg to about 5 mg, or about 3 mg of magnesium stearate. In certain embodiments, the push layer can further comprise from about 1 mg to about 5 mg, about 2 mg, about 3 mg, or about 4 mg of color pigment.

In certain embodiments, Seal Coat-1 can comprise from about 20 mg to about 50 mg, about 25 mg, about 30 mg, about 35 mg, or about 40 mg of a hydroxypropyl cellulose based polymer (OPADRY® EZ clear). In certain embodiments, Seal Coat-2 can comprise from about 1 mg to 15 mg, about 5 mg, or about 15 mg of a hydroxypropyl cellulose based polymer (OPADRY® EZ clear).

In certain embodiments, Functional Coat/membrane can comprise from about 100 mg to about 200 mg, from about 125 mg to about 175 mg, from about 145 mg to about 150 mg, about 111.2 mg, about 129.7 mg, or about 148 mg of a copolymer od ethyl acrylate, methyl methacrylate, and trimethylammonioethyl methacrylate chloride (1:2:0.2) with a Tg of between about 60° C. and about 70° C. (EUDRAGIT® RL PO). In certain embodiments, the Functional Coat/membrane can further comprise from about 10 mg to about 30 mg, from about 15 mg to about 25 mg, about 16.7 mg, about 19.4 mg, or about 22.20 mg of triethyl citrate. In certain embodiments, the Functional Coat can further comprise from about 20 mg to about 40 mg, about 22.2 mg, about 25.9 mg, or about 29.6 mg of talc.

In certain embodiments, the gastroretentive tablets can comprise an immediate release (IR) drug layer comprising CD, LD, hydroxypropyl cellulose, α-tocopherol, and succinic acid. In certain embodiments, the IR drug layer can comprise from about 10 mg to about 20 mg, about 13.5 mg, or about 17.5 mg of CD. In certain embodiments, the IR drug layer can comprise from about 50 mg to about 75 mg, or about 65 mg of LD. In certain embodiments, the IR drug layer can further comprise from about 10 mg to about 20 mg, about 11.6 mg, or about 15 mg of hydroxypropyl cellulose. In certain embodiments, the IR drug layer can further comprise from about 0.1 mg to about 1 mg, about 0.4 mg, or about 0.5 mg of α-tocopherol. In certain embodiments, the IR drug layer can further comprise from about 1 mg to about 5 mg, about 2.5 mg, or about 3.25 mg of succinic acid.

In certain embodiments, the gastroretentive tablets are finally coated with a Cosmetic Coat/Over Coat. In certain embodiments, the Cosmetic Coat/Over Coat can comprise from about 15 mg to about 20 mg, about 15 mg, about 17 mg, or about 20 mg of OPADRY® II Pink, OPADRY® II Green, or OPADRY® II Blue.

### 6.3. Methods of Treating

In certain embodiments, the disclosure provides methods for treating PD, comprising administering self-regulating, oral, osmotic, floating gastroretentive compositions of CD and LD. The gastroretentive CD/LD compositions of the disclosure provide and maintain steady therapeutic plasma concentrations of LD and are superior to the marketed extended release CD/LD compositions approved by the FDA for the treatment of PD. PD patients on such dosage forms wake up in the morning having little or no mobility (off-time) due to the wearing off of the dose taken the day/evening before. Once the previous dose has worn off, the patients are usually unwilling, or even unable, to wait for the extended period of time required for an extended release dosage form to deliver the necessary plasma levels of LD. While the use of an immediate release formulation of LD can reduce this “wait time”, the use of an immediate release formulation of LD requires more frequent dosing and is associated with more fluctuating plasma LD concentrations. The gastroretentive CD/LD compositions of the disclosure provide extended release, with reduced lag time, and steady therapeutic plasma concentrations of LD. The gastroretentive compositions of the disclosure, due to the presence of a permeable elastic membrane and a push-pull osmotic core, can provide steady delivery of a moderately soluble drug, e.g., LD, because the permeable elastic membrane allows for gastric retention and passive diffusion of the drug, and the push-pull system provides an additional thrust to expel the drug as drug concentration decreases over time.

In certain embodiments, the disclosure provides methods for treating Parkinson's disease, and reduce “off” periods and LD induced dyskinesias, comprising administering self-regulating, oral, osmotic, floating gastroretentive CD/LD compositions.

In certain embodiments, the disclosure provides methods for treating post-encephalitic parkinsonism, and reduce “off” periods and LD induced dyskinesias, comprising administering self-regulating, oral, osmotic, floating gastroretentive CD/LD compositions.

In certain embodiments, the disclosure provides methods for treating parkinsonism that may follow carbon monoxide intoxication or manganese intoxication, comprising administering self-regulating, oral, osmotic, floating gastroretentive CD/LD compositions.

In certain embodiments, the disclosure provides methods for improving compliance in PD patients. The method comprises providing once-a-day or twice-a-day administration of self-regulating, oral, osmotic, floating gastroretentive CD/LD compositions in patients with PD. The CD/LD composition of the disclosure provide extended release with steady therapeutic plasma concentration of CD and LD for at least about 8 hours, e.g., between about 8 hours and about 14 hours, or between about 10 hours and about 14 hours. The gastroretentive CD/LD compositions of the disclosure reduce “off time”, increase “on” time without disabling dyskinesia, and reduce the severity of dyskinesia in comparison to the standard oral extended release formulations.

In certain embodiments, the disclosure provides minimizing lag time and improving compliance in PD patients. The method comprises administering to a PD patient, an oral, osmotic controlled, floating gastroretentive CD/LD composition of the disclosure containing an IR drug layer that provides immediate release of CD/LD to minimize lag time/wait time, and an extended release portion that provides extended release with steady therapeutic plasma concentration of CD and LD for at least about 8 hours, e.g., between about 8 hours and about 14 hours, or between about 10 hours and about 14 hours.

In certain embodiments, the disclosure provides method of improving bioavailability of LD. The method comprises administering to a subject, a self-regulating, oral, osmotic, floating gastroretentive CD/LD composition that can provide extended release with enhanced pharmacokinetic attributes of CD and LD, e.g., avoidance of low trough levels, and reduced peak-to-trough ratios (Cₘₐₓ/Cₘᵢₙ). The composition enhances drug solubility by releasing CD and LD in acidic microenvironment of stomach and enhances CD/LD absorption by releasing the drugs near their site of absorption. The gastroretentive CD/LD composition of the disclosure provides extended release of CD and LD for about 8 to about 14 hours, without losing gastroretentive attributes of the system (GRS attributes), and collapses after complete release of the drug from the system.

In certain embodiments, the disclosure provides a method for improving patient compliance by administering gastroretentive CD/LD compositions of the disclosure that can avoid gastric emptying and reducing peak-to-trough fluctuations generally associated with oral CD/LD dosage forms. As LD is absorbed mainly in proximal small intestine, gastric emptying plays an important role in determining plasma LD levels after intake of conventional oral formulation. Erratic gastric emptying is common in PD patients and likely contributes to fluctuations in LD plasma levels and unpredictable motor responses observed with orally dosed LD. The present invention fills this void by providing self-regulating, oral, osmotic, floating gastroretentive CD/LD compositions that provide desired pharmacokinetic attributes, i.e., substantially steady plasma concentrations/levels of LD and CD over prolonged periods of time compared to marketed CD/LD compositions. The gastroretentive oral CD/LD dosage forms of the disclosure avoid erratic fluctuations in LD plasma levels by providing a sustained release of LD in the stomach of a patient.

In certain embodiments, the disclosure provides oral, osmotic controlled, floating gastroretentive CD/LD compositions that improve oral bioavailability of CD and LD. The gastroretentive compositions of the disclosure markedly improve absorption and bioavailability of CD and LD and, in particular, improves their absorption and bioavailability in the proximal GI tract, due to its ability to withstand peristalsis and mechanical contractility of the stomach (shear, or shear effect), and consequently releases the drugs in an extended manner in the vicinity of its absorption site(s) and without premature transit into nonabsorbing regions of the GI tract. This avoids/reduces the side effects and improves patient compliance by releasing drug near the absorption site, rather than in colon, where they have a potential for altering normal gut flora and release dangerous toxins causing nausea, vomiting, and other life-threatening effects.

### 6.4. Methods of Making

In certain embodiments, the disclosure provides a method for making an osmotic, floating gastroretentive dosage form, the method comprises making a pull layer blend comprising CD/LD co-granulates and an extragranular component; making a push layer blend; compressing the pull layer blend and the push layer blend into a multilayered tablet core, coating the tablet core with a functional coat to provide a functional coated tablet core; drilling an orifice into the functional coat to provide a functional coated tablet core containing an orifice; and coating the functional coated tablet core containing an orifice with an immediate release drug layer comprising CD and LD and at least one binder. In certain embodiments, the CD/LD co-granulates comprise CD, LD, a polyethylene oxide polymer with an average molecular weight of less than or equal to 1M Da, a polyethylene oxide polymer with an average molecular weight of greater than 1M Da, an acid, at least one binder, and at least one stabilizing agent; and the extragranular component comprises at least one gas generating agent. In certain embodiments, the gas-generating agent(s) is present in intermediate drug granules and/or an extragranular component. In certain embodiments, the extragranular component can further include a filler, a glidant, and/or a lubricant. In certain embodiments, the CD/LD co-granulates comprise a polyethylene oxide polymer with an average molecular weight of about 200K Da and a polyethylene oxide polymer with an average molecular weight of about 7M Da. In certain embodiments, the polyethylene oxide polymer with an average molecular weight of about 7M Da and the polyethylene oxide polymer with an average molecular weight of about 200K Da are present in a respective weight ratio of between about 1:99 and 10:90. In certain embodiments, the push layer comprises at least one polyethylene oxide polymer with an average molecular weight of greater than or equal to 600K Da and at least one osmogen. In certain embodiments, the functional coat comprises a copolymer of ethyl acrylate, methyl methacrylate, and trimethylammonioethyl methacrylate chloride (1:2:0.2) with a glass transition temperature of between 60° C. and 70° C., and at least one plasticizer.

In certain embodiments, the pull layer comprises CD/LD co-granulates that contain CD and LD; and extragranular components, blended into a pull layer blend. In certain embodiments, CD/LD co-granulates are made via dry granulation or wet granulation. In certain embodiments, CD and LD are blended with excipients via hot-melt extrusion or spray drying to obtain a pull layer blend.

In certain embodiments, the compositions comprise a multilayered tablet core coated with a coating system containing various coats in the following order: multilayered tablet core coated with Seal Coat-1, permeable membrane/Functional Coat over Seal Coat-1, Seal Coat-2 over Permeable membrane/Functional Coat; IR drug layer over Seal Coat-2; Cosmetic Coat over IR drug layer and optionally, a Clear Coat over Cosmetic Coat. In certain embodiments, the multilayered tablet core is a bilayered tablet core.

In certain embodiments, the seal coat(s) can comprise OPADRY® II, clear; functional coat can comprise EUDRAGIT® RL PO; Cosmetic Coat can comprise OPADRY® II, Pink/Green/Blue; and the Final Coat can comprise OPADRY® EZ, clear.

In certain embodiments, the IR drug layer can comprise CD and LD for immediate release, talc, and a binder.

In certain embodiments, the coating system can include an orifice. In certain embodiments, orifice is drilled manually or is drilled with a laser. In certain embodiments, the IR drug layer, the Cosmetic Coat and the Clear Coat do not include any orifice. In certain embodiments, orifice in the coating system can be in fluid continuation with the pull layer.

### 6.5. Features of the Dosage Form

The present disclosure provides self-regulating, osmotic, floating gastroretentive CD/LD compositions. In certain embodiments, the CD/LD compositions of the disclosure release pharmaceutically effective amount of LD and CD, independent of initial concentration of the drugs. In certain embodiments, the release of CD and LD is partly through the permeable elastic membrane and partly through the orifice. In certain embodiments, the release of LD and CD from the self-regulating, osmotic, floating gastroretentive compositions is independent of various physiological factors within the GI tract. The compositions expand and swell rapidly, independent of the physiological factors in the GI tract, and can be retained in the stomach for extended periods of time, e.g., about 8 hours to about 14 hours, regardless of the stomach pH, by maintaining the tablet integrity in a swollen state, e.g., swollen state comprising a volume gain of at least about 100%, and provide extended release of LD and CD under varying hydrodynamic and pH conditions. In certain embodiments, the gastroretentive compositions of the disclosure retain a volume gain of at least about 200%, based on the volume of the dosage form at the time of contact with the GI fluid, for at least about 8 hours.

The self-regulating, osmotic, floating gastroretentive compositions of the disclosure provide extended release, with steady therapeutic plasma concentration and minimal pharmacokinetic variability, of CD and LD.

In certain embodiments, the gastroretentive compositions of the disclosure, in light meal or heavy meal conditions, swell to a size that prevents their passage through the pyloric sphincter, and the membrane maintains the integrity of the system in a swollen state for prolonged periods of time under hydrodynamic conditions created by gastric motility (shear effect) and pH variations. In certain embodiments, the gastroretentive compositions of the disclosure swell within 60 minutes or less to a size that prevents their passage through the pyloric sphincter, remain in the swollen state for at least about 8 hours, and collapse/squeeze for complete emptying through the pyloric sphincter, after at least about 80% of the drug is released. In certain embodiments, the gastroretentive compositions of the disclosure remain in the swollen state for at least about 6 hours, e.g., about 10 hours to about 24 hours. Furthermore, as the pull layer containing the active pharmaceutical agent, e.g., LD and CD, is released from the orifice and the push layer continues to swell, the dosage form becomes sufficiently empty, e.g., when at least about 80% of the active pharmaceutical agent(s) is released, and finally collapses, for complete emptying through the pyloric sphincter. In certain embodiments, the dosage form becomes sufficiently empty after at least about 70% to about 100%%, at least about 75%, at least about 80%, at least about 85%, at least about 90%, at least about 95%, at least about 100%, or intermediate values therein, of the drug is released. In certain embodiments, the oral, osmotic, controlled release, floating gastroretentive compositions of the disclosure regulate core swelling and membrane elasticity as a function of time to enable emptying of the gastroretentive composition from the stomach.

In certain embodiments, release of CD and LD from the gastroretentive compositions is independent of various physiological factors within the GI tract, and the release characteristics of the composition can be predicted from the properties of the active pharmaceutical agent and the composition. The compositions expand rapidly, independent of the physiological factors in the GI tract, and can be retained in the stomach for extended periods of time, e.g., between about 8 hours to about 24 hours, regardless of the stomach pH, by maintaining the tablet integrity in a swollen state, and provide extended release of CD and LD under varying hydrodynamic and pH conditions.

In certain embodiments, the pull layer and the push layer each contain at least one swellable hydrophilic water-soluble polymer to provide controlled drug release and prevent dose dumping.

In certain embodiments, the swellable water-soluble hydrophilic polymers, e.g., polyethylene oxide, in the push layer and the pull layer control the release of CD and LD under varying hydrodynamic and pH conditions. In certain embodiments, controlled release of CD and LD from the composition depends upon the average molecular weight of polyethylene oxide present in the pull layer, e.g., an increase in the average molecular weight of polyethylene oxide in the pull layer reduces release rate of the drug. In certain embodiments, the push layer comprises at least one polyethylene oxide having an average molecular weight of greater than about 600K Da. In certain embodiments, average molecular weight of polyethylene oxide in the push layer determines the release rate of CD and LD. In certain embodiments, an increase in the average molecular weight of polyethylene oxide in the push layer increases swelling rate and swelling volume of the polyethylene oxide with imbibition of water. In certain embodiments, an increase in average molecular weight of the polyethylene oxide in the push layer increases the release rate of the drug from the pull layer. In certain embodiments, the push layer contains a polyethylene oxide polymer with an average molecular weight of about 2M Da (POLYOX™ N60) and the pull layer contains a polyethylene oxide polymer with an average molecular weight of about 200K Da (POLYOX™ N80). In certain embodiments, the pull layer includes a polyethylene oxide with an average molecular of about 7M Da and a polyethylene oxide with an average molecular weight of about 200K Da, that are present in a weight ratio of between about 1:99 and about 10:90, respectively. In certain embodiments, the average molecular weights of the polyethylene oxides in the pull layer and the push layer are different enough to prevent mixing of the two layers and provide a decreasing viscosity gradient from the push layer to the pull layer.

In certain embodiments, swellable water-soluble hydrophilic polymers in the pull layer and the push layer of the tablet core, and a permeable elastic membrane, over the tablet core, containing an orifice in fluid communication with the pull layer, control the release of CD and LD for extended periods of time.

In certain embodiments, the gastroretentive composition includes at least one osmogen that provides concentration gradient to facilitate osmotic flow of gastric fluid into the composition. In certain embodiments, the osmogen is present in the push layer. In certain embodiments, the osmogen is present in the pull layer and the push layer. In certain embodiments, the gastroretentive compositions of the disclosure comprise a permeable membrane comprising a copolymer with high permeability, e.g., a copolymer of ethyl acrylate, methyl methacrylate, and trimethylammonioethyl methacrylate chloride (1:2:0.2), e.g., EUGRAGIT® RL copolymer, e.g., EUDRAGIT® RL PO or EUDRAGIT® RL 30D. In certain embodiments, the highly permeable EUDRAGIT RL PO copolymer is highly elastic with glass transition temperature of between about 60° C. and about 70° C., to allow for rapid swelling of the dosage form. In certain embodiments, the gastroretentive compositions of the disclosure comprise a permeable membrane comprising a highly permeable copolymer with Tg of between about 60° C. and about 70° C., e.g., EUDRAGIT® RL PO (1:2:0.2), to facilitate quick expansion of the membrane as the CO₂ gas is being generated.

In certain embodiments, the gastroretentive compositions of the disclosure exhibit a floating lag time of less than about 60 minutes, less than about 55 minutes, less than about 40 minutes, less than about 35 minutes, less than about 30 minutes, less than about 25 minutes, less than about 20 minutes, less than about 15 minutes, or any intermediate time periods therein, in a dissolution medium comprising about 0.001N HCl and about 10 mM NaCl.

In certain embodiments, the gastroretentive compositions of the disclosure exhibit a floating lag time of less than about 60 minutes, less than about 55 minutes, less than about 40 minutes, less than about 35 minutes, less than about 30 minutes, less than about 25 minutes, less than about 20 minutes, less than about 15 minutes, or any intermediate time periods therein, in pH 4.5 acetate buffer.

In certain embodiments, the oral, osmotic, controlled release, floating gastroretentive compositions of the disclosure exhibit a floating lag time of between about 30 minutes and about 60 minutes in an in vivo dissolution medium comprising GI fluids.

In certain embodiments, the floating lag time is independent of the pH of the dissolution medium.

In certain embodiments, the gastroretentive dosage forms of the disclosure exhibit a volume gain of at least about 100% in about 60 minutes or less, a volume gain of at least about 125% in about 2 hours, a volume gain of at least about 300% in about 4 hours, and collapse/squeeze to a volume gain of about 200% or less in about 16 hours, in pH 4.5 acetate buffer, measured from the time of contact with the buffer.

In certain embodiments, the gastroretentive dosage forms of the disclosure exhibit a volume gain of at least about 150% in about 60 minutes or less, a volume gain of at least about 200% in about 2 hours, a volume gain of at least about 200% in about 4 hours, and collapse/squeeze to a volume gain of about 100% or less in about 22 hours, in a dissolution medium comprising about 0.001N HCl and about 10 mM NaCl, measured from the time of contact with the dissolution medium.

In certain embodiments, the gastroretentive dosage forms of the disclosure exhibit a volume gain of at least about 200% in about 60 minutes or less, a volume gain of at least about 200% in about 2 hours, a volume gain of at least about 200% in about 4 hours, and collapse to a volume gain of about 150% or less in about 22 hours, in a dissolution medium comprising about 0.001N HCl and about 10 mM NaCl, measured from the time of contact with the dissolution medium.

In certain embodiments, the gastroretentive dosage forms of the disclosure exhibit a volume gain of at least about 100% in about 60 minutes or less, a volume gain of at least about 300% in about 2 hours, a volume gain of at least about 300% in about 4 hours, and collapse to a volume gain of about 250% or less in about 22 hours, in a dissolution medium comprising about 0.001N HCl and about 10 mM NaCl, measured from the time of contact with the dissolution medium.

In certain embodiments, the gastroretentive dosage forms of the disclosure exhibit a volume gain of at least about 100% in about 60 minutes or less, a volume gain of at least about 150% in about 2 hours, a volume gain of at least about 200% in about 4 hours, and collapse to a volume gain of about 100% or less in about 22 hours, in a dissolution medium comprising about 0.001N HCl and about 10 mM NaCl, measured from the time of contact with the dissolution medium.

In certain embodiments, the gastroretentive dosage forms of the disclosure, when coming in contact with a dissolution medium comprising 0.001N HCl and about 10 mM NaCl, exhibit a volume gain of at least about 100% in about 60 minutes or less, a volume gain of at least about 150% in about 2 hours, and collapse/squeeze to a volume gain of about 150% or less in about 22 hours, measured from measured from the time of contact with the dissolution medium.

In certain embodiments, the gastroretentive dosage forms of the disclosure, when coming in contact with a dissolution medium comprising 0.001N HCl and about 10 mM NaCl, exhibit a volume gain of at least about 100% in about 60 minutes or less, at least about 200% volume gain in about 2 hours, and collapse/squeeze to less than 200% volume gain in about 22 hours, measured from the time of contact with the dissolution medium.

In certain embodiments, the gastroretentive dosage forms of the disclosure, when coming in contact with a dissolution medium comprising 0.001N HCl and about 10 mM NaCl, exhibit a volume gain of at least about 100% in about 60 minutes or less, at least about 250% volume gain in about 2 hours, and collapse/squeeze to less than 250% volume gain in about 22 hours, measured from the time of contact with the dissolution medium.

In certain embodiments, the gastroretentive dosage forms of the disclosure, when coming in contact with a dissolution medium comprising 0.001N HCl and about 10 mM NaCl, exhibit a volume gain of at least about 100% in about 60 minutes or less, at least about 300% volume gain in about 2 hours, and collapse/squeeze to less than 300% volume gain in about 22 hours, measured from the time of contact with the dissolution medium.

In certain embodiments, the gastroretentive compositions of the disclosure markedly improve absorption and bioavailability of CD and LD and, in particular, improves their absorption and bioavailability in the proximal GI tract, due to its ability to withstand peristalsis and mechanical contractility of the stomach (shear, or shear effect), and consequently release the drugs in an extended manner in the vicinity of its absorption site(s) and without premature transit into nonabsorbing regions of the GI tract. In certain embodiments, unlike other formulations in the art that require a high calorie and high fat diet for maintaining gastric retention for up to 8-10 hours, the gastroretentive compositions of the disclosure provide gastric retention of the active pharmaceutical agents with NAW, e.g., CD and LD, for at least about 8 hours, without premature transit in nonabsorbing regions of the GI tract, in the low or medium calorie diet conditions.

In certain embodiments, presence of an orifice in the membrane prevents membrane tearing and keeps the dosage form intact for extended periods. The orifice releases excess pressure built up during swelling of the dosage form, e.g., swelling of the push layer, and allows the membrane to remain intact until at least 80% of the drug is released. In certain embodiments, the gastroretentive composition of the disclosure provides gastric retention and extended release of CD and LD for a period of between about 6-24 hours, between about 8-16 hours or between about 10 hours and about 14 hours. In certain embodiments, the gastroretentive composition of the disclosure provides gastric retention and extended release of CD and LD for up to 8, 9, 10, 11, 12, 13, 14, 15, 16, 17, 18, 19, 20, 21, 22, 23, 24 hours, or any intermediate periods therein. In certain embodiments, the gastroretentive compositions of the disclosure provide gastric retention and extended release of CD and LD for at least from about 10 to about 14 hours. In certain embodiments, the dosage form stays in a swollen state comprising a volume gain of at least about 150%, based on the volume of the dosage form when in contact with a dissolution medium, for a period of between about 8-14 fours. In certain embodiments, the dosage form stays in a swollen state comprising a volume gain of at least about 200%, based on the volume of the dosage form when in contact with a dissolution medium, for a period of between about 8-14 fours.

In certain embodiments, membrane permeability affects floating lag time and floating time of the composition. In certain embodiments, permeation of gastric fluid into the dosage form, and generation of CO₂ from the gas-generating agent, increases with increasing membrane permeability. In certain embodiments, floating lag time decreases with increasing membrane permeability. In certain embodiments the membrane comprises a highly permeable copolymer of ethyl acrylate, methyl methacrylate, and trimethylammonioethyl methacrylate chloride with a Tg of between about 60° C. and about 70° C.

Without intending to be bound by any particular theory of operation, it is believed that the presence of a swellable, water-soluble hydrophilic polyethylene oxide polymer (e.g., POLYOX®), a gas-generating agent, and an acid in the multilayered tablet core, and a water-insoluble permeable elastic membrane comprising a EUDRAGIT® RL copolymer (copolymer of ethyl acrylate, methyl methacrylate, and trimethylammonioethyl methacrylate chloride (1:2:0.2), provides a rapidly swelling/expanding extended release gastroretentive composition with desired characteristics for drug release, hydrodynamic balance, and mechanical strength to withstand pH variations and shear effect in the stomach during fed and fasted conditions.

In certain embodiments, the dosage forms of the disclosure comprise multilayered tablets that are compressed horizontally into oval, modified oval, or capsule shape for easy swallowing. In certain embodiments, it was surprisingly observed that horizontally compressed tablets provided superior gastroretentive properties compared to vertically compressed tablets. In certain embodiments, the tablets are compressed using oval, modified oval, capsule shaped or any other shaping tool. In certain embodiments, the horizontally compressed multilayered tablets comprise a major axis having a length of between about 12 mm and about 22 mm, and a minor axis having a length of between about 8 mm and about 11 mm. In certain embodiments, the multilayered tablets have a major axis of about 12 m, about 13 mm, about 14 mm, about 15 mm, about 16 mm, about 17 mm, about 18 mm, about 19 mm, about 20 mm, about 21 mm, about 22 mm, or any intermediate lengths therein. In certain embodiments, the multilayered tablets have a minor axis of about 8 m, about 9 mm, about 10 mm, about 11 mm, or any intermediate lengths therein. In certain embodiments, the horizontally compressed multilayered tablets comprise a major axis having a length of about 20±2 mm, and a minor axis having a length of between about 10±2 mm.

In certain embodiments, the initial tablet size (e.g., major axis x minor axis of about 19 mm×10 mm) is reasonably small for swallowability, and once swallowed, the tablet is designed for rapid generation of carbon dioxide (CO₂) within the core to increase the buoyancy. In certain embodiments, the tablets, within 30 minutes of coming into contact with a simulated gastric medium, start floating and transforms into an oblong shape with major and minor axis having lengths of about 26 and 18 mm respectively, which is maintained for more than 12 hours. Once the dosage form achieves the constant size, the push-pull system gets activated and drug is released at constant rate for about 8-14 hours of duration.

In certain embodiments, the gastroretentive compositions of the disclosure, when in contact with gastric fluid, or with media that simulate gastric condition, expand within about 30-60 minutes to a size that prevents their passage through the pyloric sphincter of a human, and exhibit a floating lag time of less than about 60 minutes, e.g., less than about 45 minutes, less than about 40 minutes, less than about 40 minutes, less than about 35 minutes, less than about 30 minutes, less than about 29 minutes, less than about 28 minutes, less than about 27 minutes, less than about 26 minutes, less than about 25 minutes, less than about 24 minutes, less than about 23 minutes, less than about 22 minutes, less than about 21 minutes, less than about 20 minutes, less than about 19 minutes, less than about 18 minutes, less than about 17 minutes, less than about 16 minutes, less than about 15 minutes, less than about 14 minutes, less than about 13 minutes, less than about 12 minutes, less than about 11 minutes, less than about 10 minutes, or less than about 9 minutes. In certain embodiments, the tablet's shape and size, e.g., oval shaped horizontally compressed tablet comprising a long axis having a length of about 20±2 mm, and a short axis having a length of between about 10±2 mm, prevents its passage through the pyloric sphincter, with just 50% increase in volume of the tablet in gastric fluid.

In certain embodiments, the gastroretentive compositions of the disclosure exhibit a breaking strength of ≥15N.

In certain embodiments, the gastroretentive compositions of the disclosure exhibit a hardness of about 5 kp to about 20 kp. In certain embodiments, hardness of the bilayered tablet core is about 5 kp, about 6 kp, about 7 kp, about 8 kp, about 9 kp, about 10 kp, about 11 kp, about 12 kp, about 13 kp, about 14 kp, about 15 kp, about 16 kp, about 17 kp, about 18 kp, about 19 kp, about 20 kp, or any intermediate value therein.

In certain embodiments, the gastroretentive compositions of the disclosure are suitable for once or twice daily administration. In certain embodiments, the gastroretentive compositions of the disclosure provide extended release of CD and LD for a period of about 8-14 hours, under fed and fasted conditions.

In certain embodiments, the disclosure provides a dosage regimen comprising administering, once or twice daily to a subject in need thereof, a pharmaceutical gastroretentive composition comprising about 54 mg of CD and 200 mg of LD, 60 mg of CD and about 240 mg of LD; about 65 mg of CD and 240 mg of LD, about 70 mg of CD and about 280 mg of LD, or about 80 mg of CD and about 320 mg of LD, about 86 mg of CD and about 320 mg of LD, about 103 mg of CD and about 380 mg of LD, about 87 mg of CD and about 320 mg of LD, about 100 mg of CD and about 370 mg of LD, and about 78 mg od CD and about 290 mg of LD.

As noted above, in certain embodiments, the multilayer tablet core comprises gas-generating agents, e.g., carbonate and bicarbonate salts, that generate CO₂ in acidic environment, e.g., gastric fluid. In certain embodiments, the multilayer tablet core further comprises organic and/or inorganic acids that react with carbonate/bicarbonate salts in an aqueous environment, e.g., independent of stomach pH, and generate CO₂ gas. In certain embodiments, the membrane is highly elastic/flexible due to the presence of a highly permeable copolymer of ethyl acrylate, methyl methacrylate, and trimethylammonioethyl methacrylate chloride (1:2:0.2) and at least one plasticizer and expands rapidly with an outward pressure on the membrane from the generated CO₂ gas. In certain embodiments, the rate of swelling of the multilayer tablet core is synchronized with the rate of expansion of the membrane, such that the multilayer tablet core expands along with the expanding membrane. In certain embodiments, the tablet core swells at a rate such that the pull layer in the swollen core is facing the orifice in the expanded membrane and provides drug release through the orifice. In certain embodiments, the membrane expansion is responsible for an initial rapid expansion/swelling of the dosage form and the swellable multilayer tablet core within the membrane supports the expanded membrane.

In certain embodiments, the expanded dosage form collapses back to about 200% or less volume gain in about 16 hours or less, in about 14 hours, in about 12 hours, or intermediate values therein, based on the time of contact with a dissolution medium. In certain embodiments, the expanded dosage form collapses back to about 150% or less volume gain in about 16 hours or less, in about 14 hours, in about 12 hours, or intermediate values therein, based on the time of contact with a dissolution medium. In certain embodiments, the dosage form can squeeze due to release of drug and excipients from the tablet core, and effusion of CO₂ through the membrane into the surrounding environment.

In certain embodiments, the multilayer tablet core swells to a size that can support the expanded permeable elastic membrane. In certain embodiments, the permeable elastic membrane containing an orifice keeps the multilayer tablet core intact in a swollen condition for prolonged time periods and the dosage provides extended release of the drug for the prolonged time periods, e.g., 8-14 hours

In certain embodiments, the rate of generation of CO₂ and rate of expansion of membrane is enhanced with increasing membrane permeability. In certain embodiments, expansion of membrane is faster than swelling of tablet core. Such time differential in membrane expansion swelling of tablet core results in empty space between the tablet core and the membrane to accommodate generated CO₂, which keeps the dosage form in swollen state for long time periods and enhances its gastric residence time.

In certain embodiments, the dosage form provides extended release of CD and LD for at least about 12 hours, in about 250 ml of pH 4.5 acetate buffer, measured using BioDis reciprocating cylinder method at 25 dpm.

In certain embodiments, the dosage form provides extended release of CD and LD for at least about 12 hours, in 900 ml of pH 4.5 acetate buffer, measured using custom basket method at 100 rpm.

In certain embodiments, the dosage form provides extended release of CD and LD, for at least about 12 hours, in 200 ml of pH 4.5 acetate buffer, measured using rotating bottle method at 15 rpm.

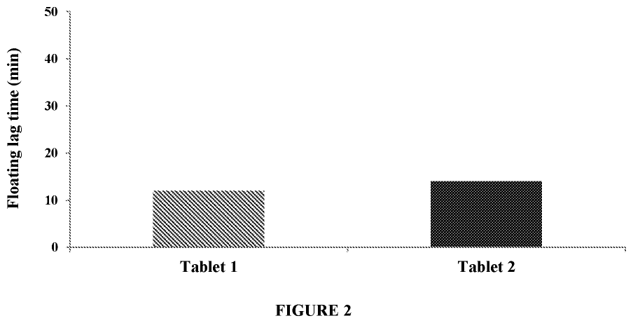

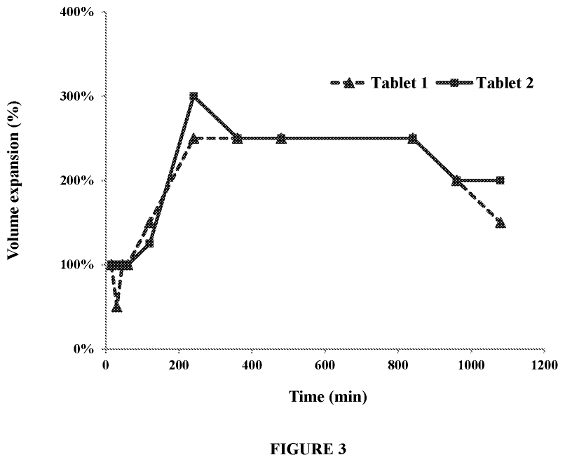

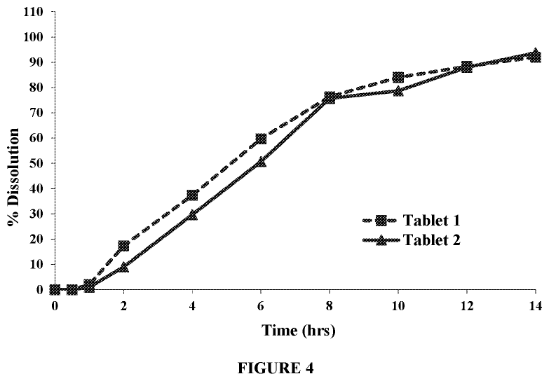

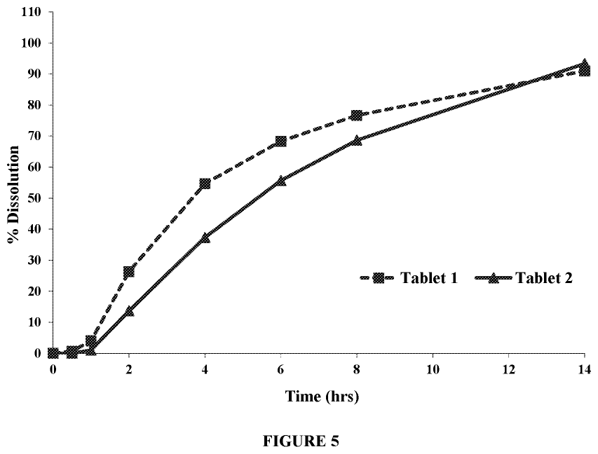

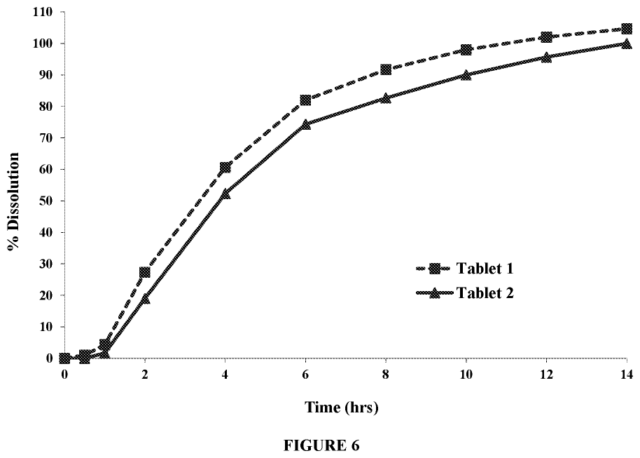

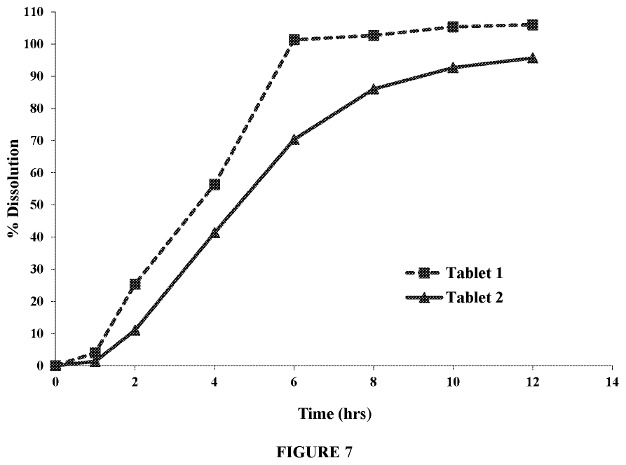

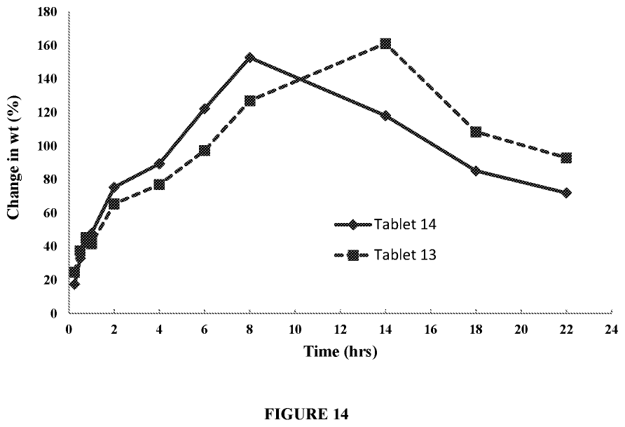

The gastroretentive compositions of the disclosure can conveniently release CD and LD, without losing bioavailability, in an extended release profile, or in a combined immediate and extended release profile. Because the gastric retention depends primarily on swelling and floating mechanisms, the swelling behavior was evaluated in terms of gravimetric swelling (water uptake) and volumetric swelling (size increase). FIGS. 3, 16, 18, 19, and 21 show swelling kinetics (volumetric) of test formulations. FIGS. 14, 15, 17, and 20 show gravimetric swelling of the test formulations. As the entrapment of in situ-generated carbon dioxide produced by the reaction between sodium bicarbonate and/or calcium carbonate with the acid and/or the SGF, floating lag time was also measured (FIG. 2). In addition, multiple tests of a tablet's ability to withstand shear forces, offering higher discrimination of the effects of such forces, were also utilized: a custom basket method at 100 rpm (FIG. 4), a rotating bottle method at 15 rpm (FIG. 5), and a BioDis reciprocating cylinder method at 25 dpm (FIGS. 6 and 7). Finally, dissolution tests were performed in dissolution mediums mimicking GI conditions in presence and absence of food, e.g., dissolution testings in pH 4.5 acetate buffer; about 0.001N HCl containing about 10 mM NaCl; 0.01 N HCl containing 150 mM NaCl; or a light meal medium comprising an aqueous medium comprising sodium chloride, potassium chloride, potassium hydrogen sulfate, calcium chloride, citric acid, and sugar. The test procedures to measure these properties are described in the Examples below.

### 7. EXAMPLES

The detailed description of the present disclosure is further illustrated by the following Examples, which are illustrative only and are not to be construed as limiting the scope of the disclosure. Variations and equivalents of these Examples will be apparent to those skilled in the art in light of the present disclosure, the drawings, and the claims herein

### Example 1: Preparation of Extended Release CD/LD Tablets

The present example provides various formulations of extended release CD/LD tablets as outlined in Tables 1-3. Fourteen different tablets were prepared.

TABLE 1
Formulations of CD/LD Tablets

| | | | | | |
|---|---|---|---|---|---|
| | Tablet 1 | Tablet 2 | Tablet 3 | Tablet 4 | Tablet 5 |
| Ingredients | mg/dose | mg/dose- | mg/dose | mg/dose | mg/dose |

Pull Layer Blend

| | | | | | |
|---|---|---|---|---|---|
| Levodopa | 200.0 | 200.0 | 240.0 | 320.0 | 240.0 |
| Carbidopa | 54.0 | 54.0 | 64.80 | 86.40 | 64.80 |
| POLYOX ^{®} N80 | 200.0 | 200.0 | 193.26 | 141.56 | 190.7 |
| POLYOX ^{®} N303 | 5.00 | 5.0 | 5.0 | 5.014 | 5.0 |
| Hydroxypropyl | 8.00 | 8.0 | 8.0 | 8.0 | 8.0 |
| cellulose | | | | | |
| Succinic acid | 50.0 | 50.0 | 50.0 | 50.0 | 125.0 |
| Alpha tocopherol | 0.50 | 0.50 | 0.5 | 0.5 | 0.5 |
| (Vit-E) | | | | | |
| Sodium | 100.0 | 100.0 | 100.0 | 100.0 | 50.0 |
| bicarbonate | | | | | |
| Calcium | 25.0 | 25.0 | 25.0 | 25.0 | 75.0 |
| carbonate | | | | | |
| PARTECK ^{®} | 44.00 | 44.0 | — | — | 51.50 |
| M200 | | | | | |
| Cab-O-Sil ^{®} | 3.5 | 3.5 | 3.5 | 3.5 | 3.5 |
| Magnesium | 10.0 | 10.0 | 10.0 | 10.0 | 10.0 |
| stearate | | | | | |

Push Layer Blend

| | | | | | |
|---|---|---|---|---|---|
| POLYOX ^{®} N60 | 220.0 | 220.0 | 220.0 | 220.0 | 220.0 |
| Sodium chloride | 25.0 | 25.0 | 25.0 | 25.0 | 25.0 |
| Red pigment | 2.0 | 2.0 | 2.0 | 2.0 | — |
| blend (PB1595) | | | | | |
| Oxide Pigment | — | — | — | — | 4.0 |
| Black (PB- | | | | | |
| 177003) | | | | | |
| Magnesium | 3.0 | 3.0 | 3.0 | 3.0 | 3.0 |
| stearate | | | | | |
| Tablet Core | 950.0 | 950.0 | 950.0 | 1000.0 | 1076.0 |
| Weight | | | | | |

Seal Coat-1

| | | | | | |
|---|---|---|---|---|---|
| OPADRY ^{®} II | 40.0 | 40.0 | 40.0 | 40.0 | 40.0 |
| clear | | | | | |

Functional Coat

| | | | | | |
|---|---|---|---|---|---|
| EUDRAGIT ^{®} RL | 111.15 | 148.2 | 111.2 | 111.2 | 111.2 |
| PO | | | | | |
| Triethyl citrate | 16.65 | 22.50 | 16.65 | 16.65 | 16.65 |
| Talc | 22.20 | 29.60 | 22.20 | 22.20 | 22.20 |
| Functional Coat | 150.0 | 200.0 | 150.0 | 150.0 | 150.0 |
| Weight Gain | | | | | |

Cosmetic Coat/Over Coat

| | | | | | |
|---|---|---|---|---|---|
| OPADRY ^{®}II, | 15.0 | 15.0 | 15.0 | 15.0 | — |
| Pink | | | | | |
| OPADRY ^{®} II, | | | | | — |
| Green | | | | | |
| OPADRY ^{®} II, | | | | | 15.0 |
| Blue | | | | | |

Final Coat

| | | | | | |
|---|---|---|---|---|---|
| OPADRY ^{®} EZ | — | | 10.0 | 10.0 | 10.0 |
| Clear | | | | | |
| Tablet Weight | 1155.0 | 1205.0 | 1165.0 | 1215.0 | 1291.0 |

TABLE 2
Formulations of CD/LD Tablets

| | | | | | |
|---|---|---|---|---|---|
| | Tablet 6 | Tablet 7 | Tablet 8 | Tablet 9 | Tablet 10 |
| Ingredients | mg/dose | mg/dose | mg/dose | mg/dose | mg/dose |

Pull Layer Blend

| | | | | | |
|---|---|---|---|---|---|
| Levodopa | 320.0 | 240.0 | 320.0 | 240.0 | 320.0 |
| Carbidopa | 86.40 | 64.80 | 86.40 | 64.80 | 86.40 |
| POLYOX ^{®} N80 | 190.6 | 190.7 | 190.6 | 190.7 | 190.6 |
| POLYOX ^{®} N303 | 5.00 | 5.0 | 5.0 | 5.0 | 5.0 |
| Hydroxypropyl | 8.00 | 8.0 | 8.0 | 8.0 | 8.0 |
| cellulose | | | | | |
| Succinic acid | 125.00 | 75.0 | 75.0 | 100.0 | 100.0 |
| Sodium Chloride | | — | — | — | — |
| α-tocopherol, | 0.50 | 0.50 | 0.50 | 0.50 | 0.50 |
| Sodium | 50.0 | 50.0 | 50.0 | 50.0 | 50.0 |
| bicarbonate | | | | | |
| Calcium | 75.0 | 75.0 | 75.0 | 75.0 | 75.0 |
| carbonate | | | | | |
| PARTECK ^{®} | — | — | — | — | — |
| M200 | | | | | |
| Cab-O-Sil | 3.5 | 3.5 | 3.5 | 3.5 | 3.5 |
| Magnesium | 10.0 | 10.0 | 10.0 | 10.0 | 10.0 |
| stearate | | | | | |

Push Layer Blend

| | | | | | |
|---|---|---|---|---|---|
| POLYOX ™ N60 | 220.0 | 220.0 | 220.0 | 220.0 | 220.0 |
| Sodium chloride | 25.0 | 25.0 | 25.0 | 25.0 | 25.0 |
| Oxide Pigment | 4.0 | 4.0 | 4.0 | 4.0 | 4.0 |
| Black (PB- | | | | | |
| 177003) | | | | | |
| Iron oxide (Red | — | — | — | — | — |
| Blend) | | | | | |
| Magnesium | 3.0 | 3.0 | 3.0 | 3.0 | 3.0 |
| stearate | | | | | |
| Tablet Core | 1126.0 | 974.5 | 1076.0 | 999.5 | 1101.0 |
| Weight | | | | | |

Seal Coat-1

| | | | | | |
|---|---|---|---|---|---|
| Leyodopa/carbid | 1126.0 | 974.5 | 1076.0 | 999.5 | 1101.0 |
| opa tablet core | | | | | |
| OPADRY ^{®} II | 40.0 | 40.0 | 40.0 | 40.0 | 40.0 |
| clear | | | | | |

Functional Coat

| | | | | | |
|---|---|---|---|---|---|
| EUDRAGIT ® | 111.2 | 111.2 | 111.2 | 111.2 | 111.2 |
| RL PO | | | | | |
| Triethyl citrate | 16.65 | 16.65 | 16.65 | 16.65 | 16.65 |
| Talc | 22.0 | 22.20 | 22.20 | 22.20 | 22.20 |
| Functional Coat | 150.0 | 150.0 | 150.0 | 150.0 | 150.0 |
| Weight Gain | | | | | |

Cosmetic Coat/Over Coat

| | | | | | |
|---|---|---|---|---|---|
| OPADRY ^{®} II, | 15.0 | — | 15.0 | — | 15.0 |
| Pink | | | | | |
| OPADRY ^{®} II, | — | 15.0 | | 15.0 | |
| Blue | | | | | |

Final Coat

| | | | | | |
|---|---|---|---|---|---|
| OPADRY ^{®} EZ | 10.0 | 10.0 | 10.0 | 10.0 | 10.0 |
| Clear | | | | | |
| Tablet Weight | 1341.0 | 1189.55 | 1291.0 | 1214.5 | 1316.0 |

TABLE 3
Formulations of CD/LD Tablets

| | | | | | |
|---|---|---|---|---|---|
| | Tablet | Tablet | Tablet | Tablet | Tablet |
| | 11 | 12 | 13 | 14 | 15 |
| Ingredients | mg/dose | mg/dose | mg/dose- | mg/dose | mg/dose |

Pull Layer Blend

| | | | | | |
|---|---|---|---|---|---|
| Levodopa | 320.0 | 315.0 | 320.01 | 320.01 | 270.0 |
| Carbidopa | 86.40 | 85.0 | 86.42 | 86.42 | 72.90 |
| POLYOX ^{®} | 190.6 | 148.0 | 189.09 | 190.6 | 189.1 |
| N80 | | | | | |
| POLYOX ^{®} | 5.0 | 5.0 | 5.0 | 5.0 | 5.0 |
| N303 | | | | | |
| Hydroxypropyl | 8.0 | 8.0 | 8.0 | 8.0 | 8.0 |
| cellulose | | | | | |
| Succinic acid | 125.0 | 50.0 | 125.0 | 125.0 | 125.0 |
| Sodium | 50 | — | — | — | — |
| Chloride | | | | | |
| α-tocopherol, | 0.50 | 0.50 | 1.98 | 1.98 | 2.00 |
| Sodium | 50.0 | 100.0 | 50.0 | 50.0 | 50.0 |
| bicarbonate | | | | | |
| Calcium | 75.0 | 25.0 | 75.0 | 75.0 | 138.5 |
| carbonate | | | | | |
| PARTECK ^{®} | — | — | — | — | — |
| M200 | | | | | |
| Cab-O-Sil | 3.5 | 3.5 | 3.5 | 3.5 | 3.5 |
| Magnesium | 10.0 | 10.0 | 13.0 | 13.0 | 13.0 |
| stearate | | | | | |

Push Layer Blend

| | | | | | |
|---|---|---|---|---|---|
| POLYOX ™ | 220.0 | 220.0 | 218.0 | 218.0 | 218.0 |
| N60 | | | | | |
| Sodium chloride | 25.0 | 25.0 | 25.0 | 25.0 | 25.0 |
| Oxide Pigment | 4.0 | | 4.0 | 4.0 | 4.0 |
| Black (PB- | | | | | |
| 177003) | | | | | |
| Iron oxide (Red | — | 2.0 | — | — | 4.0 |
| Blend) | | | | | |
| Magnesium | 3.0 | 3.0 | 3.0 | 3.0 | 3.0 |
| stearate | | | | | |
| Tablet Core | 1176.0 | 1000.0 | 1127.0 | 1127.0 | 1127.0 |
| Weight | | | | | |

Seal Coat-1

| | | | | | |
|---|---|---|---|---|---|
| Levodopa/ | 1176.0 | 1000.0 | 1127.0 | 1127.0 | 1131.0 |
| carbido | | | | | |
| pa tablet core | | | | | |
| OPADRY ^{®} II | 30.0 | 40.0 | 35.0 | 35.0 | 40.0 |
| clear | | | | | |

Functional Coat

| | | | | | |
|---|---|---|---|---|---|
| EUDRAGIT ® | 111.2 | 111.15 | 111.2 | 148.2 | 148.2 |
| RL PO | | | | | |
| Triethyl citrate | 16.65 | 16.65 | 16.60 | 22.20 | 22.20 |
| Talc | 22.20 | 22.20 | 22.20 | 29.60 | 29.60 |
| Functional Coat | 150.0 | 150.0 | 150.0 | 200.0 | 200.0 |
| Weight Gain | | | | | |

Cosmetic Coat/Over Coat

| | | | | | |
|---|---|---|---|---|---|
| OPADRY ^{®} II, | 15.0 | 15.0 | 20.0 | 20.0 | 20.0 |
| Pink | | | | | |
| OPADRY ^{®} II, | — | — | — | — | — |
| Blue | | | | | |

Final Coat

| | | | | | |
|---|---|---|---|---|---|
| OPADRY ^{®} EZ | 10.0 | — | 10.0 | 10.0 | — |
| Clear | | | | | |

IR Drug Layer

| | | | | | |
|---|---|---|---|---|---|
| Carbidopa | — | 17.55 | — | — | 13.50 |
| Levodopa | — | 65.0 | — | — | 50.0 |
| HPC | — | 15.0 | — | — | 11.60 |
| α-tocopherol, | — | 0.52 | — | — | 0.40 |
| Succinic acid | — | 3.25 | — | — | 2.5 |
| Total Weight | 1391.0 | 1306.32 | 1342.0 | 1392.0 | 1469.0 |

TABLE 4
Formulations of CD/LD Tablets

| | | | | | |
|---|---|---|---|---|---|
| | Tablet | Tablet | Tablet | Tablet | Tablet |
| | 16 | 17 | 18 | 19 | 20 |
| Ingredients | mg/dose | mg/dose | mg/dose | mg/dose | mg/dose |

Pull Layer Blend

| | | | | | |
|---|---|---|---|---|---|
| Levodopa | 320.0 | 320.0 | 240.0 | 320.0 | 240.0 |
| Carbidopa | 86.4 | 86.4 | 64.8 | 86.4 | 64.8 |
| POLYOX ^{®} | 189.1 | 189.1 | 189.2 | 189.1 | 190.0 |
| N80 | | | | | |
| POLYOX ^{®} | 5.0 | 5.0 | 5.0 | 5.0 | 5.0 |
| N303 | | | | | |
| Hydroxypropyl | 8.0 | 8.0 | 8.0 | 8.0 | 8.0 |
| cellulose | | | | | |
| Succinic acid | 125.0 | 125.0 | 125.0 | 125.0 | 125.0 |
| α-tocopherol, | 2.0 | 2.0 | 2.0 | 2.0 | 2.0 |
| Sodium | 50.0 | 50.0 | 50.0 | 50.0 | 50.0 |
| bicarbonate | | | | | |
| Calcium | 75.0 | 75.0 | 75.0 | 75.0 | 75.0 |
| carbonate | | | | | |
| PARTECK ^{®} | NA | NA | 51.5 | NA | NA |
| M200 | | | | | |
| Cab-O-Sil | 3.5 | 3.5 | 3.5 | 3.5 | 3.5 |
| Magnesium | 13.0 | 13.0 | 13.0 | 13.0 | 13.0 |
| stearate | | | | | |

Push Layer Blend

| | | | | | |
|---|---|---|---|---|---|
| POLYOX ^{™} N60 | 218.0 | 219.0 | 219.0 | 221.0 | 197.0 |
| Sodium chloride | 25.0 | 25.0 | 25.0 | 25.0 | 22.0 |
| Oxide Pigment | 4.0 | NA | NA | NA | NA |
| Black (PB- | | | | | |
| 177003) | | | | | |
| Iron oxide (Red | NA | 1.0 | 1.0 | 1.0 | 1.0 |
| pigment Blend) | | | | | |
| Magnesium | 3.0 | 3.0 | 3.0 | 3.0 | 3.0 |
| stearate | | | | | |
| Tablet Core | 1127.0 | 1125.0 | 1075.0 | 1127.0 | 1000.0 |
| Weight | | | | | |

Seal Coat-1

| | | | | | |
|---|---|---|---|---|---|
| Levodopa/ | 1127.0 | 1125.0 | 1075.0 | 1127.0 | 1000.0 |
| carbido | | | | | |
| pa tablet core | | | | | |
| OPADRY ^{®} II | 30.0 | 25.0 | 25.0 | 25.0.0 | 25.0.0 |
| clear | | | | | |

Functional Coat

| | | | | | |
|---|---|---|---|---|---|
| EUDRAGIT ® | 129.7 | 111.2 | 111.2 | 111.2 | 111.2 |
| RL PO | | | | | |
| Triethyl citrate | 19.4 | 16.7 | 16.7 | 16.7 | 16.7 |
| Talc | 25.9 | 22.2 | 22.2 | 22.2 | 22.2 |
| Functional Coat | 175.0 | 150.0 | 150.0 | 150.0 | 150.0 |
| Weight Gain | | | | | |

Seal Coat-2

| | | | | | |
|---|---|---|---|---|---|
| OPADRY ^{®} II | 5.0 | 15.0 | 15.0 | 15.0 | 15.0 |
| clear | | | | | |

IR Drug Layer

| | | | | | |
|---|---|---|---|---|---|
| Carbidopa | NA | 13.5 | 13.5 | 13.5 | 13.5 |
| Levodopa | NA | 50.0 | 50.0 | 50.0 | 50.0 |
| HPC | NA | 11.6 | 11.6 | 11.6 | 11.6 |
| α-tocopherol, | NA | 0.4 | 0.4 | 0.4 | 0.4 |
| Succinic acid | NA | 2.5 | 2.5 | 2.5 | 2.5 |

Over Coat/Cosmetic Coat

| | | | | | |
|---|---|---|---|---|---|
| OPADRY ^{®} EZ | NA | 17.0 | | 17.0 | |
| Pink | | | | | |
| OPADRY ^{®} EZ | NA | | 17.0 | | 17.0 |
| Blue | | | | | |
| Total Weight | 1337 | 1410.1 | 1360.1 | 1412.1 | 1285.1 |

Tablets 1-4 and 12 contained 100 mg of sodium bicarbonate and 25 mg of calcium carbonate, Tablets 5-11, 13, 14, and 16-20 contained 50 mg of sodium bicarbonate and 75 mg of calcium carbonate, and Tablet 15 contained 50 mg of sodium bicarbonate, and 138.5 mg of calcium carbonate. Tablets 1-4, and 12 contain 50 mg of succinic acid, Tablets 5, 6, 11, and 13-20 contained 125 mg of succinic acid, Tablets 7-8 contained 75 mg of succinic acid, and Tablets 9-10 contained 100 mg of succinic acid. Tablets 12, 15, and 17-20 further contained an IR drug layer. The IR drug layer contained CD and LD in the following amounts—Tablet 12 contained 17.55 mg of CD and 65 mg of LD, and Tablets 15 and 17-20 contained 13.5 mg of CD and 50 mg of LD. Tablets 17-20 contained Seal Coat-2 between the Functional coat and IR drug layer.

The tablets were made according to the following general procedure.

### Manufacturing Procedure:

A. Pull Layer Blend:

LD, CD, polyethylene oxide polymer with an average molecular weight of about 200K Da (POLYOX® N80), polyethylene oxide polymer with an average molecular weight of about 7M Da (POLYOX® N303), succinic acid, hydroxypropyl cellulose, and α-tocopherol were wet granulated using ethanol 200 proof or Isopropyl alcohol into CD/LD co-granulates; the CD/LD co-granulates were dried, milled, and blended with sodium bicarbonate, calcium carbonate, colloidal silicon dioxide (Cab-O-Sil®), magnesium stearate, and optionally, mannitol (PARTECK® M200), to obtain a uniform pull layer blend.

B. Push Layer Blend:

POLYOX® N60, sodium chloride, red pigment blend/oxide pigment black, and magnesium stearate were blended to obtain a uniform push layer blend.

C. Bilayered Tablet Core:

The pull layer blend from step A and push layer blend from step B were pressed horizontally, using a suitable tablet press, into a bilayered tablet core.

D. Seal Coat-1 and Functional Coat:

Bilayered tablet cores from step C were coated, using a perforated pan coater, with Seal Coat-1 comprising OPADRY® II, clear; and Functional Coat comprising triethyl citrate, EUDRAGIT® RL PO, and talc, wherein the functional coat is over Seal Coat-1.

E. Laser Hole Drilling:

A laser hole in fluid communication with the pull layer was drilled into Seal Coat-1 and Functional Coat, from step D.

F. Seal Coat-2 and OPADRY® EZ Clear

Laser hole drilled bilayered tablets from step E were coated, using a perforated pan coater, with Seal Coat-2 comprising OPADRY® II, clear (Tablets 16-20) or Final Coat comprising OPADRY® EZ, Clear (Tablets 11, 13, and 14).

G. IR Drug Layer:

Bilayered tablets from step F were coated, using a perforated pan coater, with an IR drug layer comprising CD, LD, hydroxypropyl cellulose (HPC), dl-α-tocopherol, and succinic acid.

H. Over Coat/Cosmetic Coat and Final Coat:

Laser hole drilled tablets from step E were further coated, with a Cosmetic Coat comprising OPADRY® II, Pink/Green/Blue; and optionally, a Final Coat comprising OPADRY® EZ, clear. Tablets with IR drug layer from step G were further coated with a Cosmetic Coat comprising OPADRY® II, Pink/Green/Blue. All the coatings were performed using a perforated pan coater.

### Example 2: Measurement of Volumetric Swelling

Tablet volume was determined to calculate volumetric expansion. To calculate the volume, swollen tablet was placed in a graduated measuring cylinder filled with fixed volume of dissolution medium, and the rise in dissolution medium level was noted over a 14-hour period. The percent volumetric expansion was calculated using the following equation:

$$Volumetric Gain ( \% ) = \frac{{V}_{s} - {V}_{d}}{{V}_{d}} \times 100$$

Vₛ is the volume of swollen tablet (at specific time point), and V_{d} is the volume of dry tablet (initial).

FIG. 3 compares volumetric swelling of Tablet 1 and Tablet 2 in a dissolution medium comprising about 200 ml of pH 4.5 acetate buffer, using rotating bottle dissolution method, at about 15 rpm and about 37° C. Tablet 2 contained higher coating weight gain (about 15 wt % of the uncoated tablet core) of functional coat, than Tablet 1 (about 12 wt % of the uncoated tablet core). FIG. 3 shows volume gain of Tablets 1 and 2, measured from their initial volume at the time of contact with the dissolution medium, over a 20-hour period. The figure demonstrates that the tablets swelled with a volume gain of about 100% in less than 1 hour, e.g., about 45 minutes.

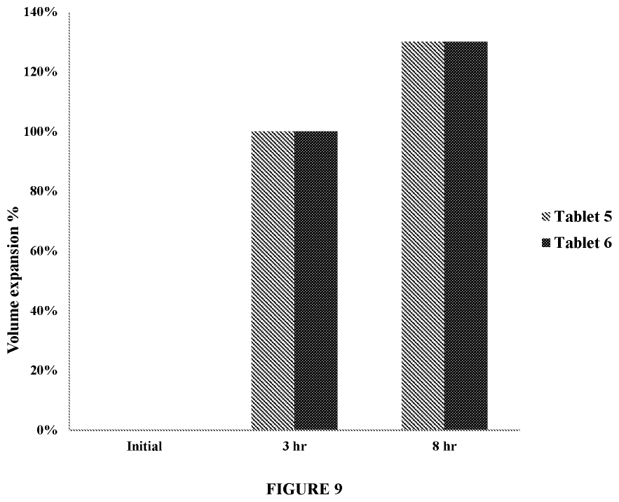

FIG. 9 compares volumetric swelling of Tablets 5 (240 mg LD) and 6 (320 mg LD) in a light meal medium comprising about 200 ml of an aqueous medium comprising sodium chloride, calcium chloride, phosphate salts, citric acid, and sugar, from their initial volume at the time of contact with the light meal medium, using rotating bottle dissolution method, at about 15 rpm and about 37° C. FIG. 9 shows volume gain of Tablets 5 and 6 over an 8-hour period. The figure demonstrates that Tablets 5 and 6 swelled with a volume gain of about 100% in about 3 hours.

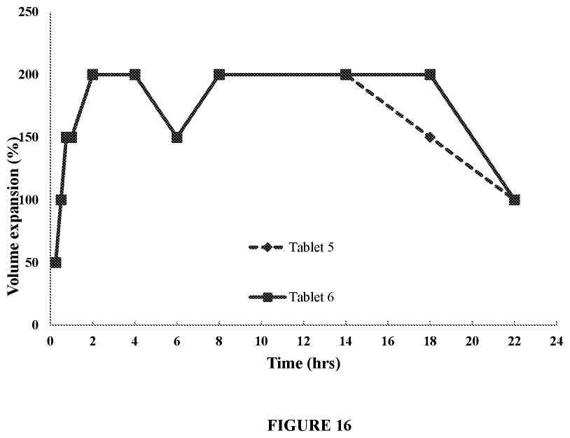

FIG. 16 compares volumetric swelling of Tablets 5 and 6 in a dissolution medium comprising about 200 ml of about 0.001N HCl and about 10 mM NaCl, measured from their initial volume at the time of contact with the dissolution medium, using a rotating bottle method, at about 15 rpm and about 37° C. Tablets 5 and 6 contained a functional coating weight gain of about 150 mg, based on the total weight of the tablet before the functional coating. FIG. 16 shows volume gain of Tablets 5 and 6 over a 22-hour period. FIG. 16 demonstrates that Tablets 5 and 6 swelled with a volume gain of about 100% in less than 1 hour; a volume gain of about 200% in about 2 hours; maintained the volume gain of about 200% for about 22 hours; and finally collapsed/squeezed to about 100% volume gain in about 22 hours.

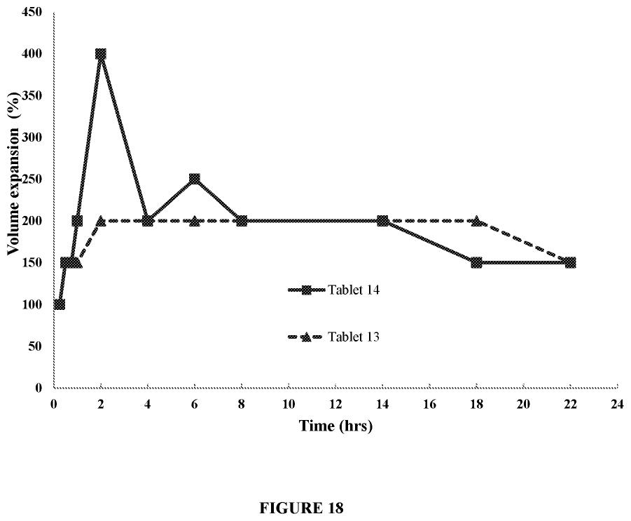

FIG. 18 compares volumetric swelling of Tablets 13 and 14 in a dissolution medium comprising about 200 ml of about 0.001N HCl and about 10 mM NaCl, measured from their initial volume at the time of contact with the dissolution medium, using a rotating bottle method, at about 15 rpm and about 37° C. Tablet 13 contained a functional coat weight gain of about 150 mg, based on the total weight of the tablet before the functional coating. Tablet 14 contained a functional coating weight gain of about 200 mg, based on the total weight of the tablet before the functional coating. FIG. 18 shows volume gain of Tablets 13 and 14 over a 22-hour period. FIG. 18 demonstrates that Tablet 13 swelled with a volume gain of about 100% in less than 1 hour, a volume gain of about 200% in about 2 hours; maintained the volume gain of about 200% for about 18 hours; and finally collapsed/squeezed to about 150% volume gain in about 22 hours. Similarly, Tablet 14 swelled with a volume gain of about 100% in less than about 1 hour, a volume gain of about 400% in about 2 hours, a volume gain of about 200% from about 4 hours to about 18 hours and collapsed/squeezed to about 150% volume gain in about 22 hours.

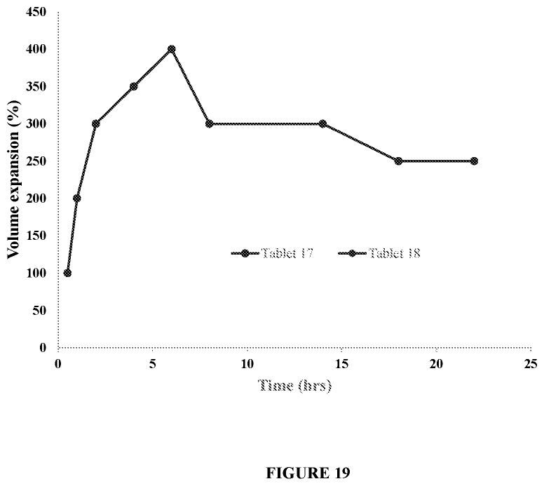

FIG. 19 compares volumetric swelling of Tablets 17 and 18 in a dissolution medium comprising about 200 ml of about 0.001N HCl and about 10 mM NaCl, measured from their initial volume at the time of contact with the dissolution medium, using a rotating bottle method, at about 15 rpm and about 37° C. Tablets 17 and 18 contained a functional coating weight gain of about 150 mg, based on the total weight of the tablet before the functional coating. FIG. 19 shows volume gain of Tablets 17 and 18 over a 22-hour period. FIG. 19 demonstrates that Tablets 17 and 18 swelled with a volume gain of at least about 100% in about 30 minutes; a volume gain of about 200% in about 1 hour; a volume gain of at least about 300% from about 2 hours to about 14 hours; and finally collapsed/squeezed to about 250% volume gain from about 14 hours to about 22 hours.

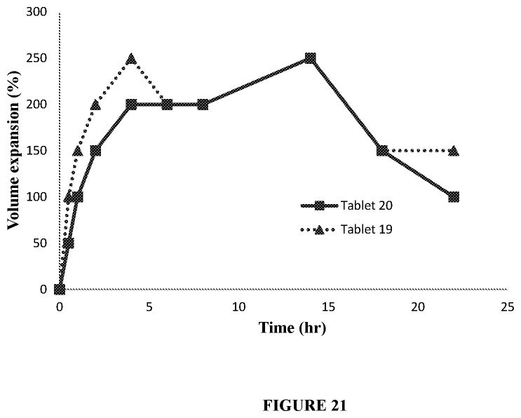

FIG. 21 compares volumetric swelling of Tablets 19 and 20 in a dissolution medium comprising about 200 ml of about 0.001N HCl and about 10 mM NaCl, measured from their initial volume at the time of contact with the dissolution medium, using a rotating bottle method, at about 15 rpm and about 37° C. Tablets 19 and 20 contained a functional coating weight gain of about 150 mg, based on the total weight of the tablet before the functional coating. FIG. 21 shows volume gain of Tablets 19 and 20 over a 22-hour period. FIG. 21 demonstrates that Tablets 19 and 20 swelled with a volume gain of at least about 100% in about one hour; a volume gain of at least about 200% in about 4 hours; a volume gain of about 250% in about 14 hours; and finally collapsed/squeezed to about 100% volume gain in about 22 hours.

### Example 3: Measurement of Floating Lag Time

The time required for the tablet to float in gastric medium is an important measure of the gastric retention, as a rapid progression to floating reduces the chance of accidental emptying (escape) of the dosage form from the stomach. The Final Coated tablets from Example 1 (Tablets 1 and 2) were placed in about 250 mL of pH 4.5 acetate buffer in a USP dissolution apparatus III-BioDis at about 25 dpm. The tablets were carefully observed until they began to float on the surface of the medium. The elapsed time was recorded and reported as floating lag time.

FIG. 2 compares floating lag time of Tablet 1 and Tablet 2 in about 250 ml of pH 4.5 acetate buffer, using USP dissolution apparatus III-BioDis reciprocating cylinder, at about 25 dpm and about 37° C. Tablet 2 contained higher coating weight gain (about 15 wt % of the uncoated tablet core weight) of functional coat, than Tablet 1 (about 12 wt % of the uncoated tablet core weight). FIG. 2 demonstrates that the tablets provided a floating lag time of about 12 minutes or less, measured from the time of contact with the dissolution medium.

Floating lag times of Tablets 5 and 6 were determined in about 200 ml of a dissolution medium comprising about 0.001N HCl and about 10 mM NaCl, using a rotating bottle method, at about 15 rpm and about 37° C. Tablets 5 and 6 contained a functional coating weight gain of about 150 mg, based on the total weight of the tablet before the functional coating. Tablets 5 and 6 provided a floating lag time of less than 20 minutes from the time of contact with the dissolution medium. Tablet 5 provided a floating lag time of about 12 minutes and Tablet 6 provided a floating lag time of about 17 minutes, measured from the time of contact with the dissolution medium.

Floating lag times of Tablets 13 and 14 were determined in about 200 ml of a dissolution medium comprising about 0.001N HCl and about 10 mM NaCl, using a rotating bottle method, at about 15 rpm and about 37° C. Tablet 13 contained a functional coating weight gain of about 150 mg, based on the total weight of the tablet before the functional coating. Tablet 14 contained a functional coat weight gain of about 200 mg, based on the total weight of the tablet before the functional coating. Tablets 13 and 14 provided a floating lag time of less than 25 minutes. Tablet 13 provided a floating lag time of 20 minutes or less, measured from the time of contact with the dissolution medium. Tablet 14 provided a floating lag time of about 25 minutes, measured from the time of contact with the dissolution medium.

Floating lag times of Tablets 19 and 20 were determined in about 200 ml of a dissolution medium comprising about 0.001N HCl and about 10 mM NaCl, using a rotating bottle method, at about 15 rpm and about 37° C. Tablets 19 and 20 contained a functional coating weight gain of about 150 mg, based on the total weight of the tablet before the functional coating. Tablets 19 and 20 provided a floating lag time of less than 45 minutes. Tablet 19 provided a floating lag time of about 37 minutes and Tablet 20 provided a floating lag time of about 16 minutes, measured from the time of contact with the dissolution medium.

### Example 4: Measurement of Dissolution Profile

Dissolution of drug from the dosage form is an important measure to achieve controlled and extended delivery of the drug. Dissolution studies were performed using different conditions to assess the effect of different physiological and hydrodynamic conditions with regards to pH, buffer, and shear forces. The United States Pharmacopeia (USP) has established standardized dissolution apparatus to measure the in vitro performance of a drug product for development and quality control purposes. These standard procedures use in vitro solubility as a surrogate for in vivo absorption. Because of the floating nature of the tablet, USP dissolution apparatus I, which uses a basket as sample holder, was used to evaluate the release of drug from these tablets as a function of time. In addition, to simulate the effect of shear conditions in fasting and fed states, dissolution studies were also performed using rotating bottle dissolution method, and USP dissolution apparatus III— BioDis reciprocating cylinder method. Different dissolution methods used for this purpose are described below:

### USP Dissolution Apparatus I (Custom Basket):

A Distek Automatic Dissolution Apparatus equipped with custom size basket was used. The dissolution test was performed in about 900 mL of pH 4.5 acetate buffer to simulate fed conditions. A rotation speed of about 100 rpm was used. The drug release was measured using high performance liquid chromatography (HPLC). Samples of dissolution medium (5 ml) containing CD and LD were withdrawn at specified time intervals of 2, 4, 6, 8, 10, 12, and 14 hours, and LD content was measured by HPLC. FIG. 4 compares dissolution profiles of LD from Tablet 1 and Tablet 2 using USP dissolution apparatus I-custom basket in about 900 ml of pH 4.5 acetate buffer, at about 100 rpm. The figure demonstrates that Tablets 1 and 2 provides about 10% dissolution of LD, in a dissolution medium simulating fed state of an individual, in about 2 hours from the time of contact with the dissolution medium.

### Rotating Bottle Method:

A rotating bottle method was used to simulate high shear conditions in stomach. Tablet 1 and Tablet 2 were placed in about 200 ml of dissolution medium in a glass bottle containing about 10 g of glass beads (3 mm). The bottle was secured in the rotating arm of an apparatus placed inside a constant temperature water bath maintained at about 37° C. The bottle was rotated at speeds of about 15 rpm or about 30 rpm to simulate the effect of different shear conditions in the stomach in fed state. Samples of dissolution medium (about 5-10 ml) containing CD and LD were withdrawn at specified time intervals of 2, 4, 6, 8, and 14 hours, and LD content was measured using HPLC. FIG. 5 compares dissolution profiles of LD from Tablet 1 and Tablet 2 using rotating bottle method, in about 200 ml of pH 4.5 acetate buffer, and at about 15 rpm. The figure demonstrates that Tablets 1 and 2 provided about 10% dissolution of LD, in a dissolution medium simulating fed state of an individual, in about 2 hours from the time of contact with the dissolution medium.

### USP III (BioDis Reciprocating Cylinder Method):

A reciprocating cylinder method, associating the hydrodynamics of rotating bottle method with the facility for exposing the dosage form to different dissolution media and agitation speeds, was used to simulate high shear conditions in stomach. The dosage unit was inserted into an internal cylinder, consisting of a glass tube closed at both ends with plastic caps containing a screen. The internal cylinder was connected to metallic rod that undertook immersion and emersion movements (reciprocating action) within the dissolution vessel/external cylinder. An anti-evaporation system was deployed over the vessels in order to avoid alteration in the volume of the dissolution medium during the assay. FIG. 6 compares dissolution profiles of LD from Tablet 1 and Tablet 2, in about in 250 ml of pH 4.5 acetate buffer, using USP III-BioDis reciprocating cylinder, at about 5 dpm and about 37° C. Samples of dissolution medium containing CD and LD were withdrawn at specified time intervals of 2, 4, 6, 8, and 14 hours and drug concentrations were measured using HPLC. Tablet 2 contained higher coating weight gain (about 15 wt % of the uncoated tablet core) of functional coat than Tablet 1 (about 12 wt % of the uncoated tablet core). The figure demonstrates that Tablets 1 and 2 provided about 10% dissolution of LD, in a dissolution medium simulating a fed state of an individual, in less than about 120 minutes from the time of contact with the dissolution medium.

FIG. 7 shows cyclic dissolution profile of LD from Tablet 1 and Tablet 2 using USP dissolution apparatus III-BioDis, simulating gastric conditions during a 12-hour period, e.g., fed state, fasted state, followed by fed state (each state for four hours). FIG. 7 shows cyclic dissolution profile of LD from Tablet 1 and Tablet 2, with an initial dissolution in 250 ml pH 4.5 acetate buffer, followed by dissolution in 250 ml 0.01 N HCl, and final dissolution in 250 ml pH 4.5 acetate buffer (each dissolution period of about 4 hours). Tablet 2 contained higher coating weight gain (about 15 wt % of the uncoated tablet core) of functional coat, than Tablet 1 (about 12 wt % of the uncoated tablet core).

### Example 5: Measurement of Dissolution Profile in a Dissolution Medium Containing about 0.001 N HCL and about 10 mM NaCl

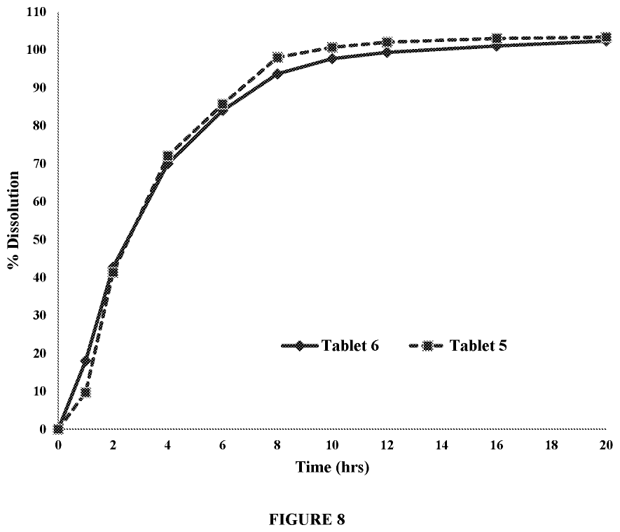

Following dissolution studies were performed using a dissolution medium comprising about 0.001 N HCL and about 10 mM NaCl. FIG. 8 compares dissolution profiles of LD from Tablet 5 (240 mg LD) and Tablet 6 (320 mg LD), in about 900 ml of a dissolution medium comprising about 0.001 N HCl and about 10 mM NaCl, using USP I-Custom basket, at about 100 rpm and about 37° C. The dissolution medium samples containing CD and LD were withdrawn at specified time intervals of 1, 2, 4, 6, 8, 10, 12, 16, and 20 hours and LD concentration was measured using HPLC. FIG. 8 demonstrates that Tablets 5 and 6 provided at least about 40% dissolution of LD in about 120 minutes from the time of contact with the dissolution medium.

### Example 6: Measurement of Dissolution Profile in a Dissolution Medium Containing about 0.01 N HCL and about 150 mM NaCl

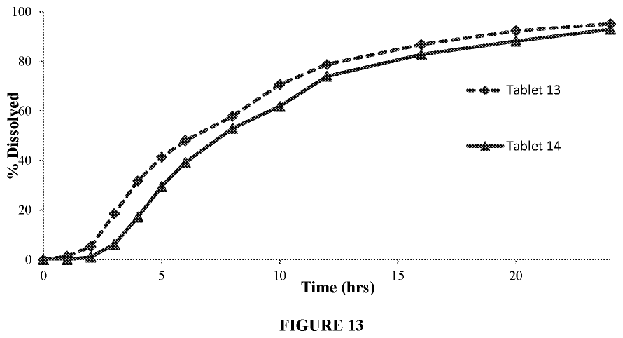

Following dissolution studies were performed using a dissolution medium comprising 0.01 N HCL and about 150 mM NaCl. FIG. 13 compares dissolution profiles of LD from Tablet 13 (320 mg LD and 150 mg functional coat weight gain) and Tablet 14 (320 mg LD and 200 mg functional coat weight gain), in 900 ml of a dissolution medium comprising 0.01 N HCl and 150 mM NaCl, using USP I-Custom basket, at about 100 rpm and about 37° C. Samples of the dissolution medium containing CD and LD were withdrawn at specified time intervals of 2, 3, 4, 5, 6, 8, 12, 16, 20, and 24 hours, and LD concentrations was measured using HPLC. FIG. 13 demonstrates that Tablet 13 provided about 35% dissolution of LD in about 4 hours, and Tablet 14 provided about 17% dissolution of LD in about 4 hours from the time of contact with the dissolution medium.

### Example 7: Gravimetric Swelling of the Compositions of the Disclosure

Tablet weights were determined to calculate the % wt gain, measured from the time of contact with a dissolution medium. Tablet weights were determined before and after placing the tablets in a dissolution medium comprising about 200 ml of about 0.001N HCl and about 10 mM NaCl, using a rotating bottle method, at about 15 rpm and about 37° C.

FIG. 14 compares gravimetric expansion of Tablets 13 and 14, in a dissolution medium comprising about 200 ml of about 0.001N HCl and about 10 mM NaCl, measured as % weight increase from the form at the time of contact with the dissolution medium, using Rotating Bottle method, at about 15 rpm and about 37° C. FIG. 14 demonstrates that Tablet 13 increased in weight by about 127% in about 8 hours and Tablet 14 increased in weight by about 153% in 8 hours.

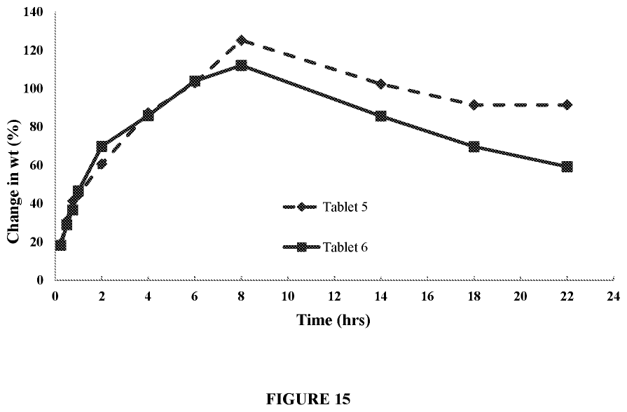

FIG. 15 compares gravimetric expansion of Tablets 5 and 6, in a dissolution medium comprising 200 ml of about 0.001N HCl and about 10 mM NaCl, measured as % weight increase from the time of contact with the dissolution medium, using Rotating Bottle method, at about 15 rpm and about 37° C. Tablet 5 contained about 240 mg of LD, about 64.80 mg of CD, and abut 51.50 mg of PARTECK® M200. Tablet 6 contained about 320 mg of LD, about 86.40 mg of CD, and no PARTECK® M200. Tablets 5 and 6 contained about equinormal amounts of succinic acid and gas-generating agent (Sodium bicarbonate and calcium carbonate mixture); and contained a coating weight gain of about 150 mg in their Functional Coat. FIG. 15 demonstrates that Tablet 5 increased in weight by about 125% in about 8 hours and Tablet 6 increased in weight by about 112% in about 8 hours.

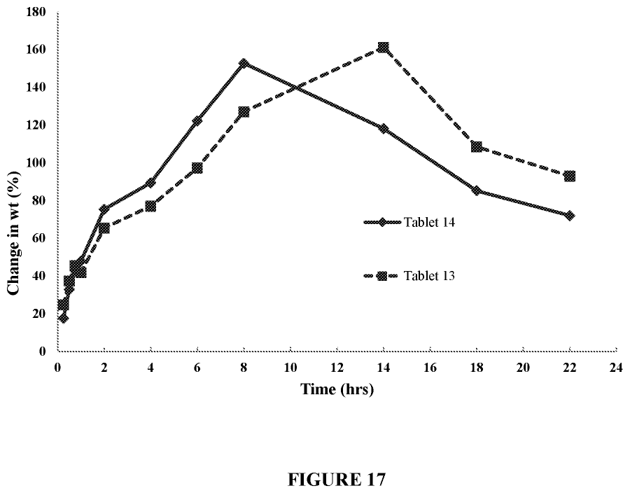

FIG. 17 compares gravimetric expansion of Tablets 13 and 14, in a dissolution medium comprising about 200 ml of about 0.001N HCl and about 10 mM NaCl, measured as % weight increase from the time of contact with the dissolution medium, using Rotating Bottle method, at about 15 rpm and about 37° C. Tablet 13 contained a functional coat weight gain of about 150 mg, based on the total weight of the tablet before the functional coating. Tablet 14 contained a functional coating weight gain of about 200 mg, based on the total weight of the tablet before the functional coating. FIG. 17 demonstrates that Tablet 13 increased in weight by about 1²⁷% in about 8 hours, about 161% in about 14 hour, about 108% in about 18 hours, and about 93% in about 22 hours; and Tablet 14 increased in weight by about 153% in about 8 hours, about 118% in about 14 hours, about 85% at about 18 hours, and about 72% in about 22 hours.

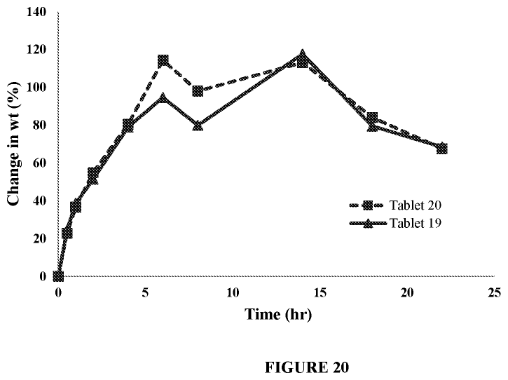

FIG. 20 compares gravimetric expansion of Tablets 19 and 20, in a dissolution medium comprising about 200 ml of about 0.001N HCl and about 10 mM NaCl, measured as % weight increase from the time of contact with the dissolution medium, using Rotating Bottle method, at about 15 rpm and about 37° C. Tablet 19 contained about 86.4 mg of CD and about 320 mg of LD; and Tablet 20 contained about 64.8 mg of CD and about 240 mg of LD. FIG. 20 demonstrates that Tablet 20 increased in weight by about 114% in about 6 hours and 68% in about 22 hours; and Tablet 19 increased in weight by about 95% in about 6 hours and 68% in about 22 hours.

### Example 8: Oral Bioavailability of CD and LD for Tablet 1 and Tablet 2

A single dose pharmacokinetic (PK) study was conducted in healthy volunteers under the fed condition to evaluate the PK performance of oral, osmotic, controlled release, floating gastroretentive dosage forms of the disclosure using Tablet 1 and Tablet 2. An open-label, single dose, cross-over comparative bioavailability study was conducted in 24 normal, healthy, adult, human subjects under high-fat high-calorie breakfast condition.

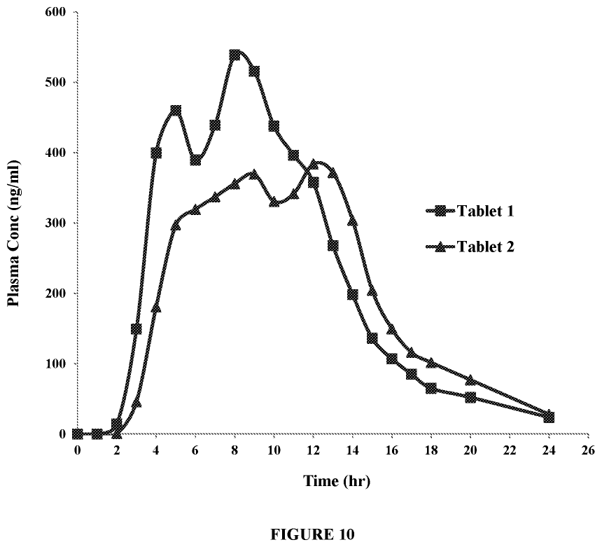

FIG. 10 provides mean (n=24) plasma concentration curves for LD. An extended release providing therapeutic concentration, from about 300 ng/ml to about 500 ng/ml, of LD for a period of about 9 hours was observed in all 24 volunteers dosed with Tablets 1 and 2. Pharmacokinetic parameters for CD and LD are summarized in Tables 4 and 5 respectively.

TABLE 4
Pharmacokinetics of CD

| Pharmacokinetic | Mean ± SD (CV %) (N = 24) | |
|---|---|---|
| parameters (units) | Tablet 1 | Tablet 2 |
| Cₘₐₓ (ng/mL) | 43.38 ± 14.89 | 37.76 ± 17.73 |
| | (34.33) | (46.95) |
| AUC₀₋ₜ | 340.70 ± 87.83 | 300.21 ± 119.25 |
| (ng · hr/mL) | (25.78) | (39.72) |
| AUC_{0-inf} | 373.33 ± 85.69 | 421.03 ± 426.59 |
| (ng · hr/mL) | (22.95) | (101.32) |
| Tₘₐₓ (hr)* | 5.00 | 11.53 |
| | (4.00-14.00) | (5.00-15.00) |
| Kₑₗ (hr − 1) | 0.21 ± 0.09 | 0.20 ± 0.10 |
| | (42.17) | (49.58) |
| t_{1/2} (hr) | 4.38 ± 3.08 | 10.47 ± 27.59 |
| | (70.33) | (263.60) |
| AUC Extrapolated | 8.94 ± 9.22 | 13.90 ± 21.40 |
| (%) | (103.19) | (154.01) |

TABLE 5
Pharmacokinetics of LD

| Pharmacokinetic | Mean ± SD (CV %) (N = 24) | |
|---|---|---|
| parameters (units) | Tablet 1 | Tablet 2 |
| Cₘₐₓ (ng/mL) | 730.36 ± 202.07 | 618.20 ± 201.33 |
| | (27.67) | (32.57) |
| AUC₀₋ₜ | 5164.54 ± 957.55 | 4505.34 ± 1481.74 |
| (ng · hr/mL) | (18.54) | (32.89) |
| AUC0−inf | 5372.20 ± 978.34 | 4987.96 ± 2415.12 |
| (ng · hr/mL) | (18.21) | (48.42) |
| Tₘₐₓ (hr)* | 8.00 | 9.00 |
| | (4.00-13.00) | (5.00-14.00) |
| Kₑₗ (hr − 1) | 0.31 ± 0.13 | 0.29 ± 0.11 |
| | (41.49) | (36.09) |
| t_{1/2} (hr) | 2.87 ± 1.87 | 3.65 ± 5.57 |
| | (64.98) | (152.39) |
| AUC Extrapolated | 3.54 ± 7.64 | 4.81 ± 13.79 |
| (%) | (215.87) | (286.68) |

The data from this study (Table 4 and Table 5/FIG. 10) demonstrates that oral, osmotic, controlled release, floating gastroretentive compositions of the disclosure (Tablet 1 and Tablet 2) provided extended release of the drug for a period of about 12 hours and were suitable for once or twice daily administration. Tablet 1 and Tablet 2, based on a twice-a-day dosing and extended release profile of over 12 hours, can be superior over non-gastroretentive formulations in reducing percentage “off” time from baseline as well as increasing percentage “on” time without troublesome dyskinesia during waking.

### Example 9: Oral Bioavailability of CD and LD for Tablet 5 and Tablet 6

A single dose pharmacokinetic (PK) study was conducted in healthy volunteers under the fed condition to evaluate the PK performance of oral, osmotic, floating gastroretentive dosage forms of the disclosure using Tablet 5 and Tablet 6. An open-label, nonrandomized, single-dose, two-treatment, one-way crossover, comparative bioavailability study was conducted in 24 normal, healthy, adult, human subjects under high-fat high-calorie breakfast condition.

Pharmacokinetic parameters for CD and LD are summarized in Tables 6 and 7, respectively.

TABLE 6
Pharmacokinetics of CD

| Pharmacokinetic | Mean ± SD (CV %) (N = 24) | |
|---|---|---|
| parameters (units) | Tablet 5 (64.80 mg) | Tablet 6 (86.40 mg) |
| Cₘₐₓ (ng/mL) | 138.14 ± 43.78 | 127.41 ± 29.06 |
| | (31.70) | (22.81) |
| AUC₀₋ₜ | 722.78 ± 175.89 | 896.46 ± 231.76 |
| (ng · hr/mL) | (24.34) | (25.85) |
| AUC_{0-inf} | 746.71 ± 176.30 | 919.28 ± 233.98 |
| (ng · hr/mL) | (23.61) | (25.45) |
| Tₘₐₓ (hr)* | 4.35 ± 0.28 | 4.80 ± 1.20 |
| | (6.434) | (25.05) |
| Kₑₗ (hr − 1) | 0.21 ± 0.08 | 0.22 ± 0.06 |
| | (36.54) | (26.42) |
| t_{1/2} (hr) | 3.73 ± 1.46 | 3.37 ± 0.71 |
| | (39.98) | (21.11) |
| AUC Extrapolated | 3.36 ± 2.18 | 2.60 ± 1.18 |
| (%) | (64.89) | (45.30) |

TABLE 7
Pharmacokinetics of LD

| Pharmacokinetic | Mean ± SD (CV %) (N = 24) | |
|---|---|---|
| parameters (units) | Tablet 5 (240 mg) | Tablet 6 (320 mg) |
| Cₘₐₓ (ng/mL) | 1566.50 ± 350.75 | 2068.05 ± 500.17 |
| | (22.39) | (24.19) |
| AUC₀₋ₜ | 8549.60 ± 981.76 | 11628.01 ± 2430.91 |
| (ng · hr/mL) | (11.48) | (20.91) |
| AUC_{0-inf} | 8612.11 ± 981.40 | 11702.07 ± 2457.26 |
| (ng · hr/mL) | (11.40) | (21.00) |
| Tₘₐₓ (hr)* | 4.41 ± 1.32 | 4.78 ± 1.53 |
| | (29.91) | (39.96) |
| Kₑₗ (hr − 1) | 0.28 ± 0.07 | 0.28 ± 0.07 |
| | (25.33) | (25.15) |
| t_{1/2} (hr) | 2.63 ± 0.62 | 2.60 ± 0.57 |
| | (23.68) | (21.97) |
| AUC Extrapolated | 0.73 ± 0.52 | 0.62 ± 0.38 |
| (%) | (70.78) | (60.85) |

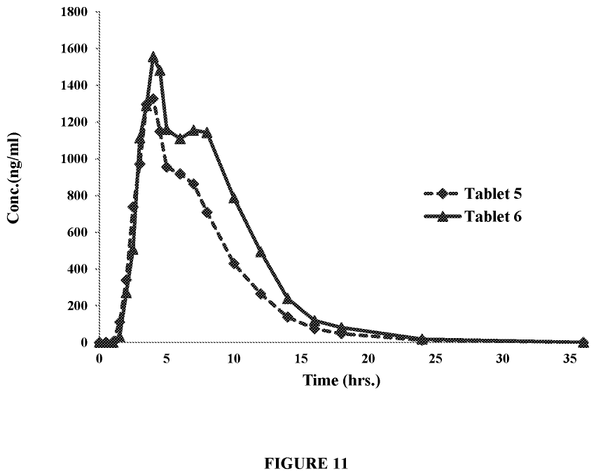

The data from this study (Table 6 and Table 7/FIG. 11) demonstrates that self-regulating, osmotic, floating gastroretentive compositions of the disclosure (Tablet 5 and Tablet 6) provided about 30% more bioavailability compared to Tablets 1 and 2. FIG. 11 provides mean (n=24) plasma concentration curves for LD. FIG. 11 demonstrates that Tablet 5 and Tablet 6 provided extended release of at least about 400 ng/ml of LD for a period of about 7 hours and about 10 hours, respectively. FIG. 11 further demonstrates dose proportionality between the 240 mg and 320 mg tablet strengths.

### Example 10: MRI Study Showing Self-Regulation of Gastroretentive Dosage Forms

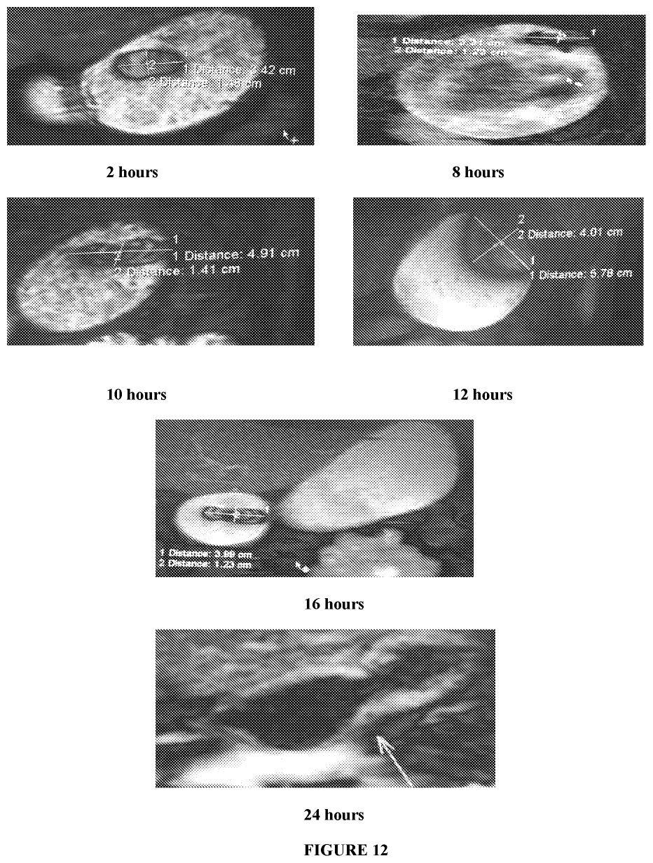

An open label, single-treatment, single period, Magnetic Resonance Imaging (MRI) study of Tablet 5 (CD/LD—60 mg/240 mg extended release tablet containing black iron oxide as MRI contrasting agent) was conducted using Siemens Magnetom Symphony 1.5 Tesla system. The study was conducted in healthy adult subjects under fed conditions. Abdominal MRI scans of stomach and intestine of the subjects were performed to see the fate of the tablet in the subjects at 8, 10, 12, 16, and 24 hours (±30 minutes) post-dose period. The tablets were visible as black spots/holes in the stomach due to the presence of black iron oxide. FIG. 12 shows post-dose MRI scan of stomach and intestine of one of the subjects consuming the dosage form. FIG. 12 shows that the black spot had spread in the entire stomach at 24 hours, indicating the tablet broke at some time between 16 hours and 24 hours post-dose.

The present disclosure is not to be limited in scope by the specific embodiments described herein. Indeed, various modifications of the disclosure in addition to those described herein will become apparent to those skilled in the art from the foregoing description. Moreover, the scope of the present disclosure is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions, and protocols are cited throughout this application, the disclosures of which are incorporated herein by reference in their entireties for all purposes.

1.-32. (canceled)

33. An osmotic, floating gastroretentive dosage form comprising:
a) a multilayer core comprising:
(i) a pull layer comprising carbidopa or a pharmaceutically acceptable salt thereof, levodopa or a pharmaceutically acceptable salt thereof,
and a gas-generating agent; and
(ii) a push layer,
b) a permeable elastic membrane containing at least one orifice and surrounding the multilayer core,
wherein the permeable elastic membrane comprises a copolymer of ethyl acrylate, methyl methacrylate, and trimethylammonioethyl methacrylate chloride,
wherein the orifice in the permeable elastic membrane is in fluid communication with the pull layer, and
wherein the copolymer is present in an amount of greater than 60 wt %, based on the total weight of the permeable elastic membrane.

34. The dosage form of claim 33, wherein the dosage form, when coming in contact with a dissolution medium, swells in 60 minutes or less to a swollen state that prevents its passage through pyloric sphincter, and collapses/squeezes for complete emptying through the pyloric sphincter, after at least about 80% of the carbidopa and the levodopa is released.

35. The dosage form of claim 33, wherein the dosage form, when coming in contact with gastric fluid, swells within 60 minutes or less to a swollen state that prevents its passage through pyloric sphincter, remains in the swollen state for at least about 8 hours, and collapses/squeezes for complete emptying through the pyloric sphincter, after at least about 80% of the carbidopa and the levodopa is released.

36. The dosage form of claim 34, wherein the dissolution medium comprises about 0.001N HCl and about 10 mM NaCl.

37. The dosage form of claim 36, wherein the dosage form, when coming in contact with the dissolution medium comprising about 0.001N HCl and about 10 mM NaCl, swells in 60 minutes or less to a swollen state that prevents its passage through pyloric sphincter, and collapses/squeezes for complete emptying through the pyloric sphincter, after at least about 80% of the carbidopa and the levodopa is released.

38. The dosage form of claim 36, wherein the dosage form, when coming in contact with the dissolution medium comprising about 0.001N HCl and about 10 mM NaCl, exhibits a volume gain of at least about 100% in about 60 minutes or less, a volume gain of least about 150% in about 2 hours, and collapses/squeezes to a volume gain of less than 150% in about 22 hours, from the time of contact with the dissolution medium.

39. The dosage form of claim 36, wherein the dosage form, when coming in contact with the dissolution medium comprising about 0.001N HCl and about 10 mM NaCl, remains in the swollen state for at least about 8 hours, from the time of contact with the dissolution medium.

40. The dosage form of claim 36, wherein the dosage form, when coming in contact with the dissolution medium comprising about 0.001N HCl and about 10 mM NaCl, floats in about 45 minutes or less, and swells in 60 minutes or less to a swollen state that prevents its passage through pyloric sphincter.

41. The dosage form of claim 36, wherein the dosage form, when coming in contact with the dissolution medium comprising about 0.001N HCl and about 10 mM NaCl, exhibits a volume gain of at least about 200% in about 60 minutes or less, and collapse to a volume gain of 150% or less in about 22 hours, from the time of contact with the dissolution medium.

42. The dosage form of claim 36, wherein the dosage form, when coming in contact with the dissolution medium comprising about 0.001N HCl and about 10 mM NaCl, swells in 60 minutes or less to a swollen state that prevents its passage through pyloric sphincter, and collapses/squeezes for complete emptying through the pyloric sphincter, after at least about 80% of the drug is released.

43. The dosage form of claim 33, wherein the membrane further comprises a plasticizer selected from the group consisting of triethyl citrate, triacetin, polyethylene glycol, propylene glycol, dibutyl sebacate, and mixtures thereof.

44. The dosage form of claim 33, wherein the core further comprises an acid selected from the group consisting of succinic acid, citric acid, malic acid, fumaric acid, stearic acid, tartaric acid, boric acid, benzoic acid, and mixtures thereof.

45. The dosage form of claim 33, wherein the pull layer and the push layer each comprises at least one water-soluble hydrophilic polymer.

46. The dosage form of claim 45, wherein the water-soluble hydrophilic polymer in the push layer is a polyethylene oxide polymer having an average molecular weight of greater than or equal to 600K Da.

47. The dosage form of claim 46, wherein the polyethylene oxide polymer has an average molecular weight of about 600K Da, about 700K Da, about 800K Da, about 900K Da, about 1M Da, about 2M Da, about 3M Da, about 4M Da, about 5M Da, about 6M Da, about 7M Da, or intermediate values therein.

48. The dosage form of claim 45, wherein the water-soluble hydrophilic polymer in the pull layer is a mixture of a polyethylene oxide polymer having an average molecular weight less than or equal to 1M Da and a polyethylene oxide polymer with an average molecular weight of greater than 1M Da.

49. The dosage form of claim 48, wherein the water-soluble hydrophilic polymer in the pull layer is a mixture of a polyethylene oxide polymer having an average molecular weight of about 7M Da and a polyethylene oxide polymer with an average molecular weight of about 200K Da.

50. The dosage form of claim 49, wherein the polyethylene oxide polymer with an average molecular weight of about 7M Da and the polyethylene oxide polymer with an average molecular weight of about 200K Da are present in a weight ratio of between 1:99 and 10:90.

51. The dosage form of claim 33, wherein the gas-generating agent is NaHCO₃, CaCO₃, or a mixture thereof.

52. The dosage form of claim 33, wherein the dosage form further comprises an immediate release drug layer comprising levodopa or a pharmaceutically acceptable salt thereof and surrounding the permeable elastic membrane.

53. The osmotic, floating gastroretentive dosage form of claim 33, wherein the dosage form is a horizontally compressed oval shaped bilayer tablet comprising a long axis with at length of between about 12 mm and about 22 mm, and a short axis with a length of between about 8 mm and about 12 mm.

54. The dosage form of claim 33, wherein the dosage form is used for the treatment of at least one movement disorder, selected from the group consisting of Parkinson's disease, post-encephalitic parkinsonism, parkinsonism resulting from injury to the nervous system by carbon monoxide or manganese intoxication, or a combination thereof.

55. The method according to claim 54 wherein the disorder is Parkinson's disease.

56. The method according to claim 54 wherein the disorder is post-encephalitic parkinsonism.

57. The method according to claim 54 wherein the disorder is parkinsonism that may follow carbon monoxide intoxication or manganese intoxication.

58. A method for improving bioavailability of levodopa or a pharmaceutically acceptable salt thereof, the method comprising orally administering to a patient in need thereof, an osmotic, floating gastroretentive dosage form comprising:
a) a multilayer core comprising:
(i) a pull layer containing carbidopa or a pharmaceutically acceptable salt thereof, levodopa or a pharmaceutically acceptable salt thereof, and a gas-generating agent; and
(ii) a push layer,
b) a permeable elastic membrane containing at least one orifice and surrounding the multilayer core,
wherein the permeable elastic membrane comprises at least one copolymer of ethyl acrylate, methyl methacrylate, and trimethylammonioethyl methacrylate chloride,
wherein the orifice in the permeable elastic membrane is in fluid communication with the pull layer,
wherein the copolymer is present in an amount of greater than 60 wt %, based on the total weight of the permeable elastic membrane.

59. A method for making an osmotic, floating gastroretentive dosage form, the method comprising:
(a) making a pull layer blend comprising carbidopa/levodopa co-granulates and an extragranular component,
(b) making a push layer blend,
(c) compressing the pull layer blend and the push layer blend into a multilayered tablet core,
(d) coating the tablet core with a functional coat to provide a functional coated tablet core, and
(e) drilling an orifice into the functional coat to provide a functional coated tablet core containing an orifice in fluid communication with the pull layer,
(f) coating the functional coated tablet core containing an orifice with an immediate release drug layer comprising levodopa or a pharmaceutically acceptable salt thereof,
wherein the carbidopa/levodopa co-granulates comprise carbidopa or a pharmaceutically acceptable salt thereof, levodopa or a pharmaceutically acceptable salt thereof, a polyethylene oxide polymer with an average molecular weight of less than or equal to 1M Da, a polyethylene oxide polymer with an average molecular weight of greater than 1M Da, and at least one acid;
wherein the extragranular component comprises at least one gas generating agent,
wherein the push layer comprises at least one polyethylene oxide polymer with an average molecular weight of greater than or equal to 600K Da; and
wherein the functional coat comprises at least one copolymer of ethyl acrylate, methyl methacrylate, and trimethylammonioethyl methacrylate chloride, and
wherein the copolymer is present in an amount of greater than 60 wt %, based on the total weight of the functional coat.
